(19) United States
(12) Patent Application Publication
(10) Pub. No.: US 2022/0098215 A1
(43) Pub. Date: Mar. 31, 2022

(54) DUOCARMYCIN ANALOGUES

(71) Applicant: AUCKLAND UNISERVICES LIMITED, Auckland (NZ)

(72) Inventors: Ho Huat LEE, Auckland (NZ); Moana TERCEL, Auckland (NZ)

(21) Appl. No.: 17/427,030

(22) PCT Filed: Jan. 28, 2020
(86) PCT No.: PCT/IB2020/050665

§ 371 Date: Jul. 29, 2021

(30) Foreign Application Priority Data
Jan. 30, 2019 (NZ) 750224

(51) Int. Cl. C07D 498/02; A61K 31/424; A61K 47/68

(52) U.S. Cl. CPC C07D 498/02; A61K 47/6803; A61K 31/424

(57) ABSTRACT

The invention relates to 2-methylbenzoxazole compounds of formula I which are analogues of the DNA alkylating subunit of the duocarmycins. Compounds of formula I can be used in the synthesis of DNA alkylating agents and antibody-drug conjugates and related compounds. The 2-methylbenzoxaxole unit of formula I has advantageous properties in combining high cytotoxicity, low lipophilicity, and unusual aqueous stability, all of which are desirable for application as payloads in efficacious antibody-drug conjugates.

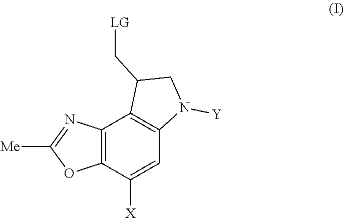

### 1. FIELD OF THE INVENTION

The present invention generally relates to 2-methylbenzoxazole compounds which can be used in the synthesis of antibody-drug conjugates (ADCs) and related compounds.

### 2. BACKGROUND TO THE INVENTION

Duocarmycins are remarkably cytotoxic natural products that bind in the minor groove of DNA and alkylate at the N3 position of adenine. The two examples below, duocarmycin SA and CC-1065 illustrate typical duocarmycin structures, which consist of a DNA-alkylating subunit, and a DNA binding subunit that binds non-covalently within the minor groove of the DNA helix.

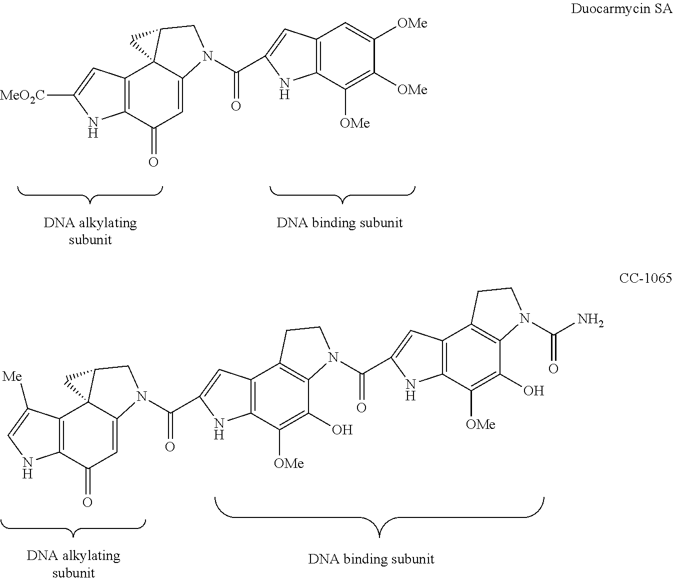 [SMILES: COC(=O)c1cc2c([nH]1)C(=O)C=C1N(C(=O)c3cc4cc(OC)c(OC)c(OC)c4[nH]3)C[C@H]3C[C@]123 ; COc1c(O)c2c(c3cc(C(=O)N4CCc5c4c(O)c(OC)c4[nH]c(C(=O)N6C[C@H]7C[C@@]78C6=CC(=O)c6[nH]cc(C)c68)cc54)[nH]c13)CCN2C(N)=O]

The mechanism of action of binding involves adenine addition to the cyclopropyl ring of the alkylating subunit, illustrated in Scheme 1 below using a general structure for this subunit. The reaction is fast and selective with DNA, but orders of magnitude slower with other nucleophiles like water, so that in the absence of DNA, the alkylating subunit persists in aqueous buffers under physiological conditions for a very long time.

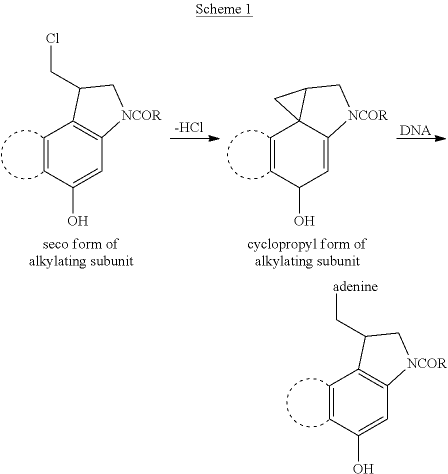

The cyclopropyl ring can be formed from a seco (i.e. ring-opened) precursor bearing a chloromethyl or other leaving group substituent, as shown in Scheme 1. The ring-closure reaction occurs so easily under physiological conditions that the two forms (seco and cyclopropyl) show essentially the same cytotoxicity. However, if the phenol of the seco alkylating subunit is in a chemical form that prevents cyclisation, cytotoxicity is greatly reduced.

The natural products such as duocarmycin SA are isolated in a single enantiomeric form, as indicated. However, both enantiomers can alkylate DNA, although the unnatural enantiomer is generally less cytotoxic.

Many synthetic versions of the duocarmycin alkylating subunit have been reported. Often these vary in the nature of the ring fusion which is indicated by the dotted line in the structures in Scheme 1 above. The nature of these ring fusions can have a very strong effect on cytotoxicity.

For example, the CBI (cyclopropabenzindole) alkylating subunit (illustrated below in the seco form in combination with the TMI (trimethoxyindole) side chain found in duocarmycin SA) generates duocarmycin analogues of similar cytotoxic potency to that of the natural products (J. Org. Chem. (1990) 55, 5823).

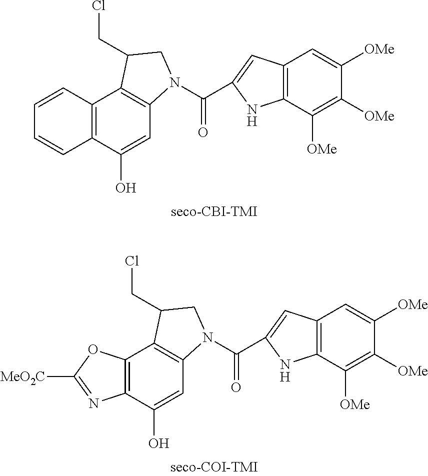 [SMILES: COc1cc2cc(C(=O)N3CC(CCl)c4c3cc(O)c3ccccc43)[nH]c2c(OC)c1OC ; COC(=O)c1nc2c(O)cc3c(c2o1)C(CCl)CN3C(=O)c1cc2cc(OC)c(OC)c(OC)c2[nH]1]

In contrast, the compound comprising the alternative COI (cyclopropaoxazoloindole) alkylating subunit in combination with TMI is several hundred-fold less cytotoxic (Bioorg. Med. Chem. Lett. (2010) 20, 1854).

Other synthetic variations have also been reported. For example, amino seco-CBIs are known, where an amine group replaces the hydroxyl group of the phenol. These variants undergo the same spirocyclisation and DNA alkylation reactions as their phenol analogues and have equivalent cytotoxic potency (ChemBioChem (2014) 15, 1998).

A further variation involves linking two alkylating subunits together in a way that allows inter-strand cross-linking of two adenines in DNA (J. Am. Chem. Soc. (1989) 111, 6428; Angew. Chem. (2010) 49, 7336). These dimers can be even more cytotoxic than the corresponding monoalkylating agents, although the activity of the dimer can not necessarily be predicted from the activity of its component monoalkylating units.

An amino seco-duocarmycin and a dimer of alkylating subunits are shown below.

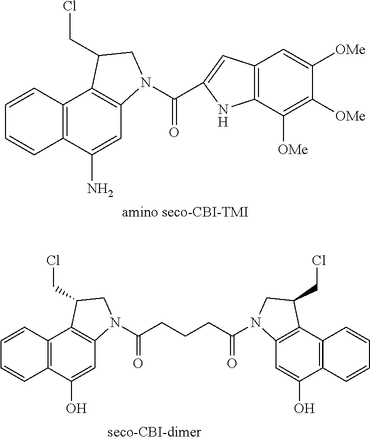 [SMILES: COc1cc2cc(C(=O)N3CC(CCl)c4c3cc(N)c3ccccc43)[nH]c2c(OC)c1OC ; O=C(CCCC(=O)N1C[C@@H](CCl)c2c1cc(O)c1ccccc21)N1C[C@@H](CCl)c2c1cc(O)c1ccccc21]

Several analogues of duocarmycin have been investigated as potential small molecule anticancer agents. However, clinical trials were not successful because toxicity limited the amounts that could be administered to very low doses.

More recently duocarmycin analogues have found application as payloads for antibody-drug conjugates (ADCs). ADCs are most frequently used in cancer therapy in which a cytotoxic small molecule (the payload) is connected via a linker to an antibody that recognises a tumour-associated antigen (Nat. Rev. Drug Discov. (2017) 16, 315; Pharmacol. Rev. (2016) 68, 3).

An ADC is constructed by chemically connecting a payload to a suitable linker, which is itself conjugated to a desired antibody. ADCs are designed to be stable in circulation but to release the payload at a predetermined target site, usually after receptor binding and internalisation into the target cell. In this way the cytotoxic action of the payload is specifically directed to the location in which damage is desirable, i.e., in a tumour. Several such ADCs have been approved as anticancer treatments.

The ADC concept is a clever means of directing a payload specifically to its target cells, thereby minimising the undesirable effects associated with conventional, non-specific systemic delivery of a toxic compound in vivo. Unfortunately, major technical challenges limit the successful application of the ADC concept.

The ADC concept limits the quantities of payloads that can be delivered to target cells, such that the payloads must be very cytotoxic for the ADC to have a therapeutic effect.

Due to their high cytotoxicity, duocarmycin analogues have been investigated as ADC payloads, both as monoalkylating agents, or as components of dimers that can cross-link DNA (see for example Mol. Pharm. (2015) 12, 1813; WO 2011/133039; Biopharm. Drug Dispos. (2016) 37, 93; Cancer Chemother. Pharmacol. (2016) 77:155-162; J. Med. Chem. (2012) 55, 766; J. Med. Chem. (2005) 48, 1344; Cancer Res. (1995) 55, 4079; Bioorg. Med. Chem. (2000) 8, 2175; WO 2018/035391; WO 2015/038426; WO 2015/153401; WO 2015/023355; WO 2017/194960; WO 2018/071455).

In the vast majority of these examples, the alkylating subunit is the highly toxic seco-CBI, as shown below.

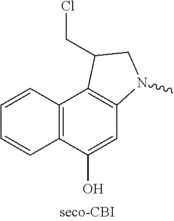

Although use of this alkylating subunit should lead to ADCs with the required cytotoxicity, seco-CBI has a significant disadvantage in being highly lipophilic. Lipophilic payloads and their derivatives are not very soluble in the aqueous buffers used to conjugate payload-linker components to antibodies, making the conjugation step difficult. The resulting ADCs are also prone to aggregation. ADC aggregates must be removed before the ADC can be used, adding complication and cost to the production of a clinically useful ADC.

Lipophilic payloads are also associated with faster clearance of ADCs from the blood stream, which reduces their overall exposure, and therefore their anti-tumour efficacy (Nat. Biotechnol. (2015) 33, 733-735).

Lipophilic payloads, particularly those that cause aggregation, can also generate ADCs that provoke an immune response in vivo, leading to unexpected toxicity or to decreased therapeutic efficacy.

However, despite the problems associated with lipophilicity, the high cytotoxicity of seco-CBI compounds continues to make them attractive payloads for anti-cancer ADCs, with various strategies employed to reduce the disadvantages.

For example, to counteract the high lipophilicity of seco-CBI some investigators have resorted to modifying the other component moieties, such that the ADC as a whole is less lipophilic. Some researchers have incorporated modified linkers (e.g. polyethylene glycol spacers, as in Mol. Pharm. (2015) 12, 1813; J. Med. Chem. (2005) 48, 1344). Others have constructed more water-soluble prodrug forms (e.g. J. Med. Chem. (2012) 55, 766, WO 2015/023355).

However, solving the payload lipophilicity issue by modifying other components of the ADC can itself have disadvantages. As well as making the synthesis of the payload-linker components more complex and time-consuming, this technique may also restrict the choice of linker.

The linker moiety attached to the payload has a considerable effect on the efficacy and safety profile of the ADC in vivo (Bioanalysis (2015) 7(13), 1561). It must be of appropriate stability in circulation but cleave in vivo when required. ADC design includes consideration of the physiological conditions in which the payload is to be released, so that the appropriate linker can be used. For example, linkers containing hydrazone moieties are pH sensitive and will cleave in lower pH environments such as endosomes and lysosomes. Disulfide linkers release the payload in a reducing environment, such as the intracellular milieu.

Some linker modifications made to mitigate undesirable lipophilic properties of the alkylating subunit may reduce the efficacy of the ADC in which they are used, such that the resulting ADC may not be the most efficacious product that could be made from a specified payload and antibody.

Other solutions to the problem of lipophilicity include ADC Bio's proprietary Lock-Release technology (www.adcbio.com) which immobilises antibodies on a solid-phase support before conjugation to the payload-linker component. Aggregation is prevented by physically separating the ADC molecules from each other during their manufacture. This allows a greater range of linkers to be used but adds additional cost to the conjugation process, without actually reducing the lipophilicity of the final ADC product.

Therefore, the lipophilicity of highly toxic alkylating subunits is a problem holding back development of new ADCs.

A further potential disadvantage of known duocarmycin analogues as ADC payloads is the high stability of the cyclopropyl form of the alkylating subunit. A stable payload that is released from an ADC into circulation may persist long enough to cause undesired systemic toxicity. Unfortunately, high stability is a feature of virtually all synthetic analogues of the CBI alkylating subunit that retain the desired potent cytotoxicity. Previous studies have shown a correlation between aqueous stability and cytotoxicity, such that the most cytotoxic analogues are stable in aqueous buffer at neutral pH (J Med Chem (2009) 52, 5271).

Accordingly, there is a need for alternative DNA-alkylating subunits that facilitate efficient synthesis of a range of ADCs while demonstrating the cytotoxicity and stability properties needed for efficacy.

It is therefore an object of the present invention to go at least some way towards meeting this need; and/or to at least provide the public with a useful choice. Other objects of the invention may become apparent from the following description which is given by way of example only.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### 3. SUMMARY OF THE INVENTION

The invention provides the novel alkylating subunit ‘2-methylbenzoxazole’ and protected and prodrug derivatives of this subunit that are suitable for attachment to a wide range of heteroaryl and aryl DNA binding moieties to produce highly cytotoxic duocarmycin analogues.

The invention also provides duocarmycin analogues comprising the novel 2-methylbenzoxazole alkylating subunit conjugated to a DNA minor groove binding unit.

These compounds can be used to produce ADCs, when bound to antibodies via linker groups.

Accordingly, in a first aspect the invention provides a compound of formula I or a pharmaceutically acceptable salt, hydrate or solvate thereof

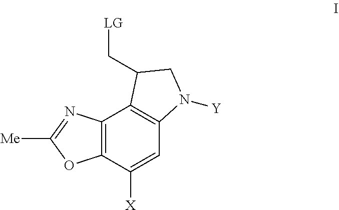

wherein:
LG is a leaving group;
X is a group selected from hydroxyl, protected hydroxyl, prodrug hydroxyl, amine, and protected amine; where amine is —NH₂, or —NH(C₁-C₆)alkyl; and
Y is a N-protecting group.

In a second aspect, the invention provides a compound of formula II or a pharmaceutically acceptable salt, hydrate or solvate thereof

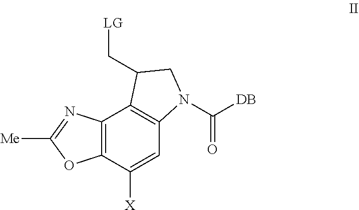

wherein:
LG is a leaving group;
X is a group selected from hydroxyl, protected hydroxyl, prodrug hydroxyl, amine, and protected amine; where amine is —NH₂, or —NH(C₁-C₆)alkyl; and
DB is a DNA minor groove binding unit.

In one embodiment, DB is an optionally substituted aryl or optionally substituted heteroaryl group attached directly or via an alkenyl group.

In one embodiment, DB is an optionally substituted indole, azaindole, benzofuran, pyridine, pyrimidine, pyrrole, imidazole, thiophene, thiazole, oxazole, pyrazole, triazole, pyrazine or pyridazine group.

In a third aspect the invention provides a compound of formula III or a pharmaceutically acceptable salt, hydrate or solvate thereof

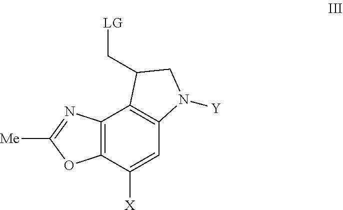

wherein:
LG is a leaving group;
X is a group selected from hydroxyl, protected hydroxyl, prodrug hydroxyl, amine, and protected amine; where amine is —NH₂, or —NH(C₁-C₆)alkyl; and

Y is selected from:
(a) a N-protecting group;
(b) —C(O)—Ar¹
(c) —C(O)—Ar¹—NH—C(O)—Ar²
(d) —C(O)—Ar¹—NH—C(O)—CH═CH—Ar³; or
(e) —C(O)—CH═CH—Ar³

wherein Ar¹, Ar² and Ar³ are each independently selected from a heteroaryl or aryl group, where each heteroaryl or aryl group is optionally substituted with one or more of —(C₁-C₆)alkyl, —CO—(C₁-C₆)alkyl, —CONH(C₁-C₆)alkyl, —CON(C₁-C₆)alkyl(C₁-C₆)alkyl, —OH, —O—(C₁-C₆)alkyl, —NH₂, —NH(C₁-C₆)alkyl, —N(C₁-C₆)alkyl(C₁-C₆)alkyl and —NHC(O)—(C₁-C₆)alkyl;

wherein in each instance —(C₁-C₆)alkyl, —CO—(C₁-C₆)alkyl, —CONH(C₁-C₆)alkyl, —CON(C₁-C₆)alkyl(C₁-C₆)alkyl, —O—(C₁-C₆)alkyl, —NH(C₁-C₆)alkyl, —N(C₁-C₆)alkyl(C₁-C₆)alkyl and —NHC(O)—(C₁-C₆)alkyl are independently optionally substituted with one or more of —NMe₂, —NHMe, —NH₂, —OH, morpholine and —SH.

In one embodiment, Ar¹, Ar² and Ar³ are independently selected from the group consisting of

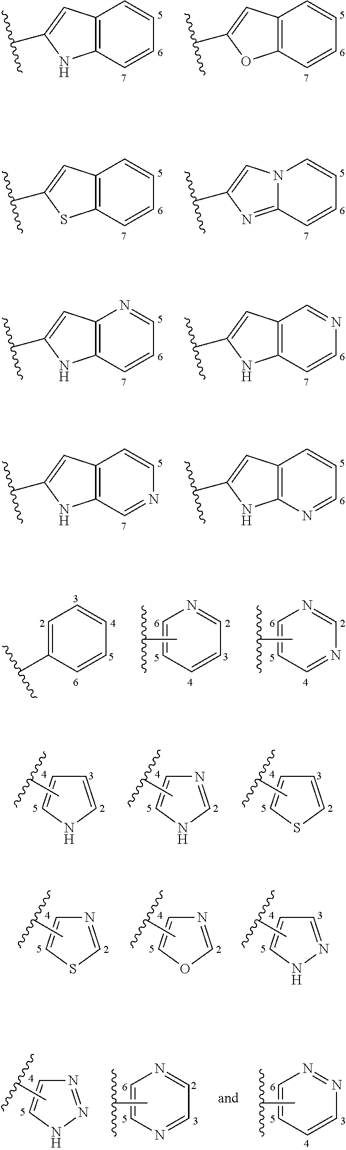

wherein [symbol] represents the point of attachment to the —C(O) or C(O)—CH═CH— group and each of the aryl or heteroaryl groups may be substituted at the numbered positions with up to three substituents selected from —(C₁-C₆)alkyl, —CO—(C₁-C₆)alkyl, —CONH(C₁-C₆)alkyl, —CON(C₁-C₆)alkyl(C₁-C₆)alkyl, —OH, —O—(C₁-C₆)alkyl, —NH₂, —NH(C₁-C₆)alkyl, —N(C₁-C₆)alkyl(C₁-C₆)alkyl and —NHC(O)—(C₁-C₆)alkyl;

wherein in each instance the substituents —(C₁-C₆)alkyl, —CO—(C₁-C₆)alkyl, —CONH(C₁-C₆)alkyl, —CON(C₁-C₆)alkyl(C₁-C₆)alkyl, —O—(C₁-C₆)alkyl, —NH(C₁-C₆)alkyl, —N(C₁-C₆)alkyl(C₁-C₆)alkyl and —NHC(O)—(C₁-C₆)alkyl may be independently optionally substituted with one or more of —NMe₂, —NHMe, —NH₂, —OH, morpholine and —SH.

In a fourth aspect the invention provides a compound of formula IV or a pharmaceutically acceptable salt, hydrate or solvate thereof

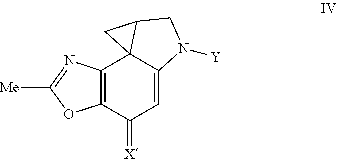

wherein V is Y or DB and X, Y and DB have the same meaning as defined for compounds of formulae I, II, and III and X′ is X with loss of H.

Compounds of formula IV may be formed through in vitro or in vivo rearrangement and concomitant elimination of H-LG from the corresponding seco-compound of formula I, II and III. All embodiments of the invention described herein for a compound of formulae I, II or III, or any of their moieties thereof are also specifically contemplated as part of the aspect of the invention that relates to compounds of formula IV, unless context dictates otherwise.

The following embodiments and preferences may relate alone or in combination of any two or more to any of the aspects of the invention set out herein.

In one embodiment LG is selected from the group consisting of chloride, bromide, iodide and —OSO₂R¹; wherein R¹ is selected from (C₁-C₁₀)alkyl, (C₁-C₁₀)heteroalkyl, (C₁-C₁₀)aryl or (C₁-C₁₀)heteroaryl.

In one embodiment LG is halo, preferably chloride, and the configuration at the chiral carbon to which LG is attached is (S).

In one embodiment X is selected from the group consisting of —OH, —OBn, —OTf, —OMOM, —OMEM, —OBOM, —OTBDMS, —OPMB, —OSEM, piperazine-1-carboxylate where the N at the 4 position is substituted with (C₁-C₁₀)alkyl, —OP(O)(OH)₂, —OP(O)(OR²)₂, —NH₂, —N═C(Ph)₂, —NHZ, NH(C₁-C₁₀)alkyl and —N—Z(C₁-C₁₀)alkyl;

wherein R² is t-Bu, Bn or allyl; and Z is selected from Boc, COCF₃, Fmoc, Alloc, Cbz, Teoc and Troc.

In one embodiment Y is a N-protecting group selected from Boc, COCF₃, Fmoc, Alloc, Cbz, Teoc and Troc.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Although the present invention is broadly as defined above, those persons skilled in the art will appreciate that the invention is not limited thereto, and that the invention also includes embodiments of which the following description gives examples.

### 4. BRIEF DESCRIPTION OF THE DRAWINGS

The invention will now be described with reference to the accompanying Figures in which:

FIG. 1 shows the LCMS analysis of the stability of seco-CBI-TMI in neutral aqueous buffer. The upper panel shows an example of the chromatogram (after an incubation time of 20 min). The lower panel summarizes the change in composition of the mixture over a total incubation time of >300 min.

FIG. 2 shows the LCMS analysis of the stability of compound 23 in neutral aqueous buffer. The upper panel shows an example of the chromatogram (after an incubation time of 200 min). The lower panel summarizes the change in composition of the mixture over a total incubation time of 500 min.

FIG. 3 shows the HPLC analysis of the stability of compound 52 in neutral aqueous buffer. The experiment was monitored hourly for a total of 8 hours.

FIG. 4 shows the HPLC analysis of the stability of compound 59 in neutral aqueous buffer. The experiment was monitored hourly for a total of 8 hours.

FIG. 5 shows the HPLC analysis of the stability of compound 66 in neutral aqueous buffer. The experiment was monitored hourly for a total of 8 hours.

### 5. DETAILED DESCRIPTION OF THE INVENTION

### 5.1 Definitions

The term “comprising” as used in this specification and claims means “consisting at least in part of”. When interpreting each statement in this specification and claims that includes the term “comprising”, features other than that or those prefaced by the term may also be present. Related terms such as “comprise” and “comprises” are to be interpreted in the same manner.

As used herein the term “and/or” means “and” or “or”, or both.

As used herein “(s)” following a noun means the plural and/or singular forms of the noun.

Asymmetric centres may exist in the compounds described herein. The asymmetric centres may be designated as (R) or (S), depending on the configuration of substituents in three-dimensional space at the chiral carbon atom. All chiral, diastereomeric and racemic forms of a structure are intended, unless a particular stereochemistry or isomeric form is indicated. All stereochemical isomeric forms of the compounds, including diastereomeric, enantiomeric, and epimeric forms, as well as d-isomers and I-isomers, and mixtures thereof, including enantiomerically enriched and diastereomerically enriched mixtures of stereochemical isomers, are within the scope of the invention.

Individual enantiomers can be prepared synthetically from commercially available enantiopure starting materials or by preparing enantiomeric mixtures and resolving the mixture into individual enantiomers. Resolution methods include (a) separation of an enantiomeric mixture by chromatography on a chiral stationary phase and (b) conversion of the enantiomeric mixture into a mixture of diastereomers and separation of the diastereomers by, for example, recrystallization or chromatography, and any other appropriate methods known in the art. Starting materials of defined stereochemistry may be commercially available or made and, if necessary, resolved by techniques well known in the art. Enantiomers having the “natural” configuration at the chiral carbon (the carbon bearing the CH₂-LG moiety in the seco form) are preferred.

The compounds described herein may also exist as conformational or geometric isomers, including cis, trans, syn, anti, entgegen (E), and zusammen (Z) isomers. All such isomers and any mixtures thereof are within the scope of the invention.

Also within the scope of the invention are any tautomeric isomers or mixtures thereof of the compounds described. As would be appreciated by those skilled in the art, a wide variety of functional groups and other structures may exhibit tautomerism. Examples include, but are not limited to, keto/enol, imine/enamine, and thioketone/enethiol tautomerism.

The compounds described herein may also exist as isotopologues and isotopomers, wherein one or more atoms in the compounds are replaced with different isotopes. Suitable isotopes include, for example, ¹H, ²H (D), ³H (T), ¹²C, ¹³C, ¹⁴C, ¹⁶, and ¹⁸O. Procedures for incorporating such isotopes into the compounds described herein will be apparent to those skilled in the art. Isotopologues and isotopomers of the compounds described herein are also within the scope of the invention.

Also within the scope of the invention are salts of the compounds described herein, including pharmaceutically acceptable salts. Such salts include, acid addition salts, base addition salts, and quaternary salts of basic nitrogen-containing groups. Acid addition salts can be prepared by reacting compounds, in free base form, with inorganic or organic acids. Examples of inorganic acids include, but are not limited to, hydrochloric, hydrobromic, nitric, sulfuric, and phosphoric acid. Examples of organic acids include, but are not limited to, acetic, trifluoroacetic, propionic, succinic, glycolic, lactic, malic, tartaric, citric, ascorbic, maleic, fumaric, pyruvic, aspartic, glutamic, stearic, salicylic, methanesulfonic, benzenesulfonic, isethionic, sulfanilic, adipic, butyric, and pivalic. Base addition salts can be prepared by reacting compounds, in free acid form, with inorganic or organic bases. Examples of inorganic base addition salts include alkali metal salts, alkaline earth metal salts, and other physiologically acceptable metal salts, for example, aluminium, calcium, lithium, magnesium, potassium, sodium, or zinc salts. Examples of organic base addition salts include amine salts, for example, salts of trimethylamine, diethylamine, ethanolamine, diethanolamine, and ethylenediamine. Quaternary salts of basic nitrogen-containing groups in the compounds may be prepared by, for example, reacting the compounds with alkyl halides such as methyl, ethyl, propyl, and butyl chlorides, bromides, and iodides, dialkyl sulfates such as dimethyl, diethyl, dibutyl, and diamyl sulfates, and the like.

The compounds described herein may form or exist as solvates with various solvents. If the solvent is water, the solvate may be referred to as a hydrate, for example, a mono-hydrate, a di-hydrate, or a tri-hydrate. All solvated forms and unsolvated forms of the compounds described herein are within the scope of the invention.

The general chemical terms used herein have their usual meanings.

Standard abbreviations for chemical groups are well known in the art and take their usual meaning, eg, Me=methyl, Et=ethyl, Bu=butyl, t-Bu=tert-butyl, Ph=phenyl, Bn=benzyl, Ac=acetyl, Boc=tert-butoxycarbonyl, Fmoc=9-fluorenylmethoxycarbonyl, Tf=triflate, OMOM=methoxymethyl ether, OMEM=methoxyethoxymethyl ether, OBOM=benzyloxymethyl ether, OTBDMS=tert-butyldimethylsilyl ether, DPPA=diphenylphosphoryl azide, NBS=N-bromosuccinimide, NIS=N-iodosuccinimide, OPMB=4-methoxybenzyl ether, EDCI=1-ethyl-3-(3-dimethylaminopropyl)carbodiimide, HOBt=hydroxybenzotriazole, OSEM=[2-(trimethylsilyl)ethoxy]methyl ether, Alloc=allyloxycarbonyl, Cbz=benzyloxycarbonyl, Teoc=(2-(trimethylsilyl)ethoxycarbonyl, TEMPO=2,2,6,6-tetramethyl-1-piperidinyloxy, Troc=2,2,2-trichlooroethylcarbonyl, and the like.

Unless stated otherwise, these abbreviations are applicable to all of the examples below.

The term “alkyl” as used herein alone or in combination with other terms, unless indicated otherwise, refers to a straight-chain or branched saturated or unsaturated acyclic hydrocarbon group. In some embodiments, alkyl groups have from 1 to 15, from 1 to 13, from 1 to 11, from 1 to 10, from 1 to 8, from 1 to 6, from 1 to 5, from 1 to 4, from 1 to 3, from 1 to 2, from 2 to 12, from 2 to 9, from 2 to 8, from 2 to 6, from 2 to 4, from 3 to 9, from 3 to 8, from 4 to 9, from 4 to 15, from 6 to 15, from 8 to 15, from 10 to 15, or 1, or 2, or 3 carbon atoms. In some embodiments, alkyl groups are saturated. Examples of such alkyl groups include but are not limited to -methyl, -ethyl, -n-propyl, -n-butyl, -n-pentyl, -n-hexyl, -n-heptyl, -n-octyl, -n-nonyl, -n-decyl, n-undecyl, n-dodecyl, n-tridecyl, n-tetradecyl, n-pentadecyl, -isopropyl, -sec-butyl, -isobutyl, -tert-butyl, -isopentyl, -neopentyl, 2-methylbutyl, -isohexyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, n-heptyl, isoheptyl, isooctyl, isononyl, isodecyl, isoundecyl, isododecyl, isotridecyl, isotetradecyl, and isopentadecyl and the like. In some embodiments, alkyl groups are unsaturated. Examples of such alkyl groups include but are not limited to -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl,-acetylenyl, -propynyl, -1-butynyl, -2-butynyl, -1-pentynyl, -2-pentynyl, -3-methyl-1-butynyl, and the like. The prefix “C-Cy”, wherein x and y are each an integer, when used in combination with the term “alkyl” refers to the number of carbon atoms in the alkyl group. In some embodiments, “alkyl” groups may be substituted with one or more optional substituents as described herein.

The term “aryl” as used herein alone or in combination with other terms, unless indicated otherwise, refers to cyclic aromatic hydrocarbon groups that do not contain any ring heteroatoms. Aryl groups include monocyclic, bicyclic and tricyclic ring systems. Examples of aryl groups include, but are not limited to, phenyl, azulenyl, heptalenyl, biphenyl, fluorenyl, phenanthrenyl, anthracenyl, indenyl, indanyl, pentalenyl, and naphthyl. In some embodiments, aryl groups have from 6 to 20, 6 to 14, 6 to 12, or 6 to 10 carbon atoms in the ring(s). In some embodiments, the aryl groups are phenyl or naphthyl. Aryl groups include aromatic-carbocycle fused ring systems. Examples include, but are not limited to, indanyl and tetrahydronaphthyl. The prefix “Cr-Cy”, wherein x and y are each an integer, when used in combination with the term “aryl” refers to the number of ring carbon atoms in the aryl group. In some embodiments, “aryl” groups may be substituted with one or more optional substituents as described herein.

The term “heteroaryl” as used herein alone or in combination with other terms, unless indicated otherwise, refers to an aromatic ring system containing 5 or more ring atoms, of which, one or more is a heteroatom. In some embodiments, the heteroatom is nitrogen, oxygen, or sulfur. A heteroaryl group is a variety of heterocyclic group that possesses an aromatic electronic structure. In some embodiments, heteroaryl groups include mono-, bi- and tricyclic ring systems having from 5 to 20, 5 to 16, from 5 to 14, from 5 to 12, from 5 to 10, from 5 to 8, or from 5 to 6 ring atoms. Heteroaryl groups include, but are not limited to pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, pyrazolopyridinyl, triazolopyridinyl, benzotriazolyl, benzoxazolyl, benzothiazolyl, imidazopyridinyl, imidazyl, isoxazolopyridinylxanthinyl, guaninyl, quinolinyl, isoquinolinyl, tetrahydroquinolinyl, quinoxalinyl, and quinazolinyl. Heteroaryl groups include fused ring systems in which all of the rings are aromatic, for example, indolyl, and fused ring systems in which only one of the rings is aromatic, for example, 2,3-dihydroindolyl. The prefix “x-y membered”, wherein x and y are each an integer, when used in combination with the term “heteroaryl” refers to the number of ring atoms in the heteroaryl group. In some embodiments “heteroaryl” groups may be substituted with one or more optional substituents as described herein.

The terms “halo” or “halogen” are intended to include F, Cl, Br, and I.

The term “heteroatom” is intended to include oxygen, nitrogen, sulfur, selenium, or phosphorus. In some embodiments, the heteroatom is selected from the group consisting of oxygen, nitrogen, and sulfur.

As used herein, the term “substituted” is intended to mean that one or more hydrogen atoms in the group indicated is replaced with one or more independently selected suitable substituents, provided that the normal valency of each atom to which the substituent/s are attached is not exceeded, and that the substitution results in a stable compound. In various embodiments, suitable optional substituents in the compounds described herein include but are not limited to halo, —(C₁-C₆)alkyl, —CO—(C₁-C₆)alkyl, —CONH(C₁-C₆)alkyl, —CON(C₁-C₆)alkyl(C₁-C₆)alkyl, —OH, —O—(C₁-C₆)alkyl, —NH₂, —NH(C₁-C₆)alkyl, —N(C₁-C₆)alkyl(C₁-C₆)alkyl, and —NHC(O)—(C₁-C₆)alkyl, —NMe₂, —NHMe, —NH₂, —OH, morpholine and —SH. The term “stable” in this context, unless indicated otherwise, refers to compounds which possess stability sufficient to allow manufacture and which maintain their integrity for a period of time sufficient to be useful for the purposes described herein.

The term “antibody” as used herein, refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cells or cells that produce autoimmune antibodies associated with an autoimmune disease. The term “antibody” includes intact monoclonal antibodies, polyclonal antibodies, multi-specific antibodies (e.g., bispecific antibodies) formed from at least two intact antibodies, and antibody fragments, so long as they exhibit the desired biological activity. Structurally, an antibody is typically a Y-shaped protein consisting of four amino acid chains, two heavy and two light. Each antibody has primarily two regions: a variable region and a constant region. The variable region, located on the ends of the arms of the Y, binds to and interacts with the target antigen. This variable region includes a complementarity determining region (CDR) that recognizes and binds to a specific binding site on a particular antigen. The constant region, located on the tail of the Y, is recognized by and interacts with the immune system (Janeway, C., Travers, P., Walport, M., Shlomchik (2001) Immuno Biology, 5^{th} Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody.

The term “reactive moiety” as used herein means a functional group that can react with a second functional group under relatively mild conditions and without the need for prior functionalisation. The reaction between the reactive moiety and second functional groups only requires the application of heat, pressure, a catalyst, acid and/or base. Examples of reactive moieties include carbamoyl halide, acyl halide, active ester, anhydride, α-haloacetyl, α-haloacetamide, maleimide, isocyanate, isothiocyanate, disulfide, thiol, hydrazine, hydrazide, sulfonyl chloride, aldehyde, methyl ketone, vinyl sulfone, halomethyl and methyl sulfonate. The second functional group will generally be a linker, or ligand.

The term “ligand” as used herein means a ligand that binds or reactively associates or complexes with a receptor, antigen or other receptive moiety associated with a given target-cell population. The ligand will generally bind via intermolecular forces such as hydrogen bonds, ionic bonds and Van der Waals forces. The ligand delivers the payload to the target cell population to which the ligand binds, generally by binding to cells expressing a particular antigen or cell surface receptor. For example, the ligand may bind to a cell surface receptor or surface protein, which is overexpressed in a diseased cell such as a cancer cell.

Examples of ligands for use in the invention include antibodies (which may be monoclonal, bi-specific, chimeric or humanized antibodies, or antibody fragments of any of these), growth factors, hormones, cell/tissue targeting peptides, aptamers and small molecules such as imaging agents, co-factors or cytokines.

The term “pharmaceutically acceptable salt”, as used herein, unless indicated otherwise, refers to pharmaceutically acceptable organic or inorganic salts of the compounds described herein. For example, the compounds described herein may contain an amino group, and accordingly acid addition salts can be formed with this amino group. Examples of salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

The terms “pharmaceutically acceptable solvate” or “solvate”, as used herein, unless indicated otherwise, refer to an association of one or more solvent molecules and a compound described herein. Examples of solvents that form pharmaceutically acceptable solvates include, but are not limited to, water, isopropanol, ethanol, methanol, DMSO, ethyl acetate, acetic acid, and ethanolamine.

### 5.2 Compounds of the Invention

The compounds of the invention provide new DNA-alkylating units with highly desirable properties, that can be utilised in the synthesis of a wide range of highly efficacious ADCs using conventional techniques.

Specifically, the compounds of the invention comprise:
(a) N-protected 2-methylbenzoxaxole DNA alkylating units (where Y is a N-protecting group) and
(b) 2-methylbenzoxaxole DNA alkylating units attached to a DNA minor groove binding unit.

The N-protected 2-methylbenzoxaxole DNA alkylating units of the invention may be readily attached to DNA minor groove binding units to provide highly cytotoxic duocarmycin analogues. They can also be conjugated to other chemical moieties to form new biologically active compounds, including ADCs.

Compounds of the invention in which the DNA alkylating subunit is attached to a DNA minor groove binding unit, can be converted to ADCs by attaching an antibody or other ligand via a linker. The linker may be attached directly to the DNA alkylating subunit via the hydroxyl or amine group X, or indirectly via the DNA minor groove binding unit at the Y position.

In a first aspect, the invention provides a compound of formula I or a pharmaceutically acceptable salt, hydrate or solvate thereof

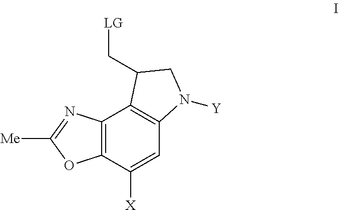

wherein:
LG is a leaving group;
X is a group selected from hydroxyl, protected hydroxyl, prodrug hydroxyl, amino, and protected amino; where amino is —NH₂, or —NH(C₁-C₆)alkyl; and
Y is a N-protecting group.

Compounds are formula I can be used in the synthesis of compounds of formula II and III, and in ADCs and their precursors.

The invention also relates to compounds of formula Ia,

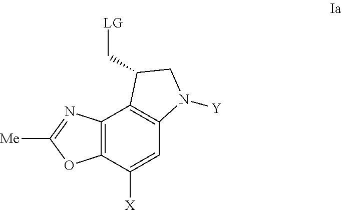

wherein LG, X and Y are as defined for formula I.

In a second aspect, the invention provides a compound of formula II or a pharmaceutically acceptable salt, hydrate or solvate thereof

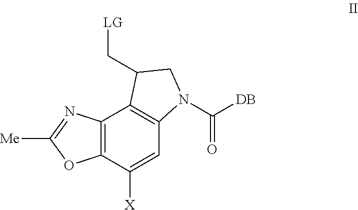

wherein:
LG is a leaving group;
X is a group selected from hydroxyl, protected hydroxyl, prodrug hydroxyl, amino, and protected amino; where amino is —NH₂, or —NH(C₁-C₆)alkyl; and
DB is a DNA minor groove binding unit.

The invention also relates to compounds of formula IIa.

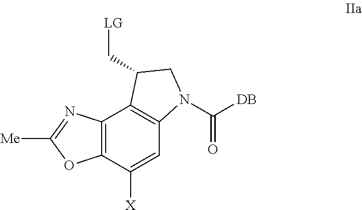

wherein LG, X and DB are as defined for formula II.

In one embodiment, DB is an optionally substituted aryl or optionally substituted heteroaryl group attached directly or via an alkenyl group.

In one embodiment, DB is an optionally substituted indole, azaindole, benzofuran, benzene, pyridine, pyrimidine, pyrrole, imidazole, thiophene, thiazole, oxazole, pyrazole, triazole, pyrazine or pyridazine group.

As discussed below, there is extensive information available in the art on the design and synthesis of DNA minor groove binding units and of ways to establish their mode and strength of DNA binding. Accordingly, a person skilled in the art can readily establish whether a particular chemical entity constitutes a DNA minor groove binding unit.

In one embodiment DB comprises a reactive moiety RM which is compatible with a complementary reactive site on a linker group, or a component of a linker group, wherein said linker group is attached to, or is suitable for attachment to a ligand, for example, an antibody.

In one embodiment, RM is a reactive moiety selected from the group consisting of azide, alkyne, carbamoyl halide, acyl halide, active ester, anhydride, α-haloacetyl, α-haloacetamide, maleimide, isocyanate, isothiocyanate, disulfide, thiol, hydrazine, hydrazide, sulfonyl chloride, aldehyde, methyl ketone, vinyl sulfone, halomethyl and methyl sulfonate.

In some embodiments of the invention, such as the compounds of formula II, the 2-methylbenzoxazole DNA alkylating subunit is bonded to a DNA minor groove binding unit (DB). Appropriate DNA binding moieties for use in the invention have an affinity for binding in the minor groove of double stranded DNA. There is extensive information available on the design and synthesis of such molecules and of ways to establish their mode and strength of DNA binding, for example as described in Drug-Nucleic Acid Interactions edited by J. B. Chaires and M. J. Waring (Methods in Enzymology Vol 340, Academic Press, 2001); Molecular Recognition of DNA by Small Molecules (Bioorg. Med. Chem. (2001) 9, 2215); Structure-Based DNA-Targeting Strategies with Small Molecule Ligands for Drug Discovery (Med. Res. Rev. (2013) 33, 1119); and A Fluorescent Intercalator Displacement Assay for Establishing DNA Binding Selectivity and Affinity (Chem. Rev. (2004) 37, 61).

The DNA binding moieties for attachment to the DNA-alkylating unit are heteroaryl or aryl groups which may be substituted with other functional groups. In general, planar aryl and hetero ring systems have appropriate physicochemical properties for binding within the minor groove, which is usually driven by a combination of H-bonding and van der Waals interactions with the DNA components of the walls and base of the groove. Aryl and heteroaryl ring substituents which enhance these interactions increase the strength of binding.

Binding is further favoured by linking together two or more ring systems, to create a longer and more extensive interaction with the minor groove, provided that the overall structure retains the correct curvature and twist to match that of the minor groove into which it binds. This is most favourably achieved when there is minimal distortion of either the small molecule ligand or the DNA; i.e. when there is a high degree of shape complementarity. The use of amide bonds to link ring systems is a favoured motif as the amide can itself participate in H-bond interactions with the DNA, while also providing sufficient flexibility to accommodate the desired twist and curvature.

A further factor for consideration, especially with DNA minor groove binders of extended length, is to maintain the correct register or positioning between H-bond donors and acceptors on the ligand and on the DNA. In some cases, this can be achieved by changing the nature of, or shifting the position of substituents on the aryl or heteroaryl rings. Extensive libraries of DNA minor groove binding ligands have been constructed and assayed for their binding affinity (e.g. Total Synthesis of Distamycin A and 2640 Analogues: A Solution-Phase Combinatorial Approach to the Discovery of New, Bioactive DNA Binding Agents and Development of a Rapid, High-Throughput Screen for Determining Relative DNA Binding Affinity or DNA Binding Sequence Selectivity, J. Am. Chem. Soc. (2000) 122, 6382) and such libraries have been applied to the preparation of duocarmycin analogues (e.g. Parallel Synthesis and Evaluation of 132 (+)-1,2,9,9a-Tetrahydrocyclopropa[c]benz[e]indol-4-one (CBI) Analogues of CC-1065 and the Duocarmycins Defining the Contribution of the DNA-Binding Domain, J. Org. Chem. (2001) 66, 6654).

In specific embodiments of the compounds of formula II, and IIa the DNA minor groove binding unit comprises a heteroaryl group which may be linked via an amide bond to a second heteroaryl group or aryl group.

In other embodiments of the compounds of formula II and IIa, the DNA minor groove binding unit comprises a single aryl or heteroaryl group linked to the DNA alkylating unit via an alkenyl group (—CH═CH—).

In a third aspect the invention provides a compound of formula III or a pharmaceutically acceptable salt, hydrate or solvate thereof

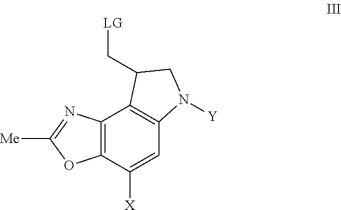

wherein:
LG is a leaving group;
X is a group selected from hydroxyl, protected hydroxyl, prodrug hydroxyl, amino, and protected amino; where amino is —NH₂, or —NH(C₁-C₆)alkyl; and

Y is selected from:
(a) a N-protecting group;
(b) —C(O)—Ar¹
(c) —C(O)—Ar¹—NH—C(O)—Ar²
(d) —C(O)—Ar¹—NH—C(O)—CH═CH—Ar³; or
(e) —C(O)—CH═CH—Ar³

wherein Ar¹, Ar² and Ar³ are each independently selected from a heteroaryl or aryl group, where each heteroaryl or aryl group is optionally substituted with one or more of —(C₁-C₆)alkyl, —CO—(C₁-C₆)alkyl, —CONH(C₁-C₆)alkyl, —CON(C₁-C₆)alkyl(C₁-C₆)alkyl, —OH, —O—(C₁-C₆)alkyl, —NH₂, —NH(C₁-C₆)alkyl, —N(C₁-C₆)alkyl(C₁-C₆)alkyl and —NHC(O)—(C₁-C₆)alkyl;

wherein in each instance —(C₁-C₆)alkyl, —CO—(C₁-C₆)alkyl, —CONH(C₁-C₆)alkyl, —CON(C₁-C₆)alkyl(C₁-C₆)alkyl, —O—(C₁-C₆)alkyl, —NH(C₁-C₆)alkyl, —N(C₁-C₆)alkyl(C₁-C₆)alkyl and —NHC(O)—(C₁-C₆)alkyl are independently optionally substituted with one or more of —NMe₂, —NHMe, —NH₂, —OH, morpholine and —SH.

The invention also relates to compounds of formula IIIa,

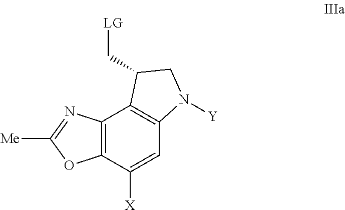

wherein LG, X and DB are as defined for formula III.

The following embodiments apply to the compounds of formula I, Ia, II, IIa, III and IIIa.

The term “leaving group” as used herein means a group that departs from a carbon centre in a substitution reaction. Usually such a group is stable in anionic form. Examples of leaving groups are well known in the art and include but are not limited to halo groups and sulfonate groups such as optionally substituted (C₁-C₆)alkanesulfonate (for example, methanesulfonate, trifluoromethanesulfonate and trifluoroethanesulfonate) and optionally substituted benzenesulfonate.

In one embodiment LG is selected from the group consisting of chloride, bromide, iodide and —OSO₂R¹; wherein R¹ is selected from (C₁-C₁₀)alkyl, (C₁-C₁₀)heteroalkyl, (C₁-C₁₀)aryl or (C₁-C₁₀)heteroaryl. In one embodiment, LG is a halide group, preferably chloride. Scheme 2 below demonstrates the synthesis of DNA alkylating subunits comprising different leaving groups.

In the compounds of the invention, the group X may be a free hydroxyl or free amino group, or may be a hydroxyl or amino group protected by a suitable protecting group (protected hydroxyl or protected amino group, respectively). X may also be a prodrug form of hydroxyl (prodrug hydroxyl).

The term “prodrug hydroxyl” means a group that is converted in vivo by the action of biochemicals such as enzymes, to provide a free OH group. Conventional procedures for the selection and preparation of suitable prodrug hydroxyl groups that can be used are described in “Design of Prodrugs”, edited by H. Bundgaard, Elsevier, 1985. Examples are well known in the art and include but are not limited to phosphate groups, carbamates and glycosides.

Compounds of the invention in which X is a prodrug hydroxyl can be used in ADCs in which the linker will attach to the DNA minor groove binding unit, rather than through X. This would result in a prodrug form of the ADC. A person skilled in the art, designing an ADC incorporating a compound of the invention, would be able to select the appropriate position at which to tether the antibody component, and determine the desirability of specific prodrug forms for the intended application of the ADC.

The term “protected hydroxyl” as used herein refers to a hydroxyl group that has been protected against undesirable reactions during synthetic procedures. The protected hydroxyl group is readily converted to the free hydroxyl group, when no longer needed and/or to allow reaction of the hydroxyl group. Hydroxyl protecting groups are described in Protective Groups in Organic Synthesis edited by T. W. Greene et al. (John Wiley & Sons, 1999). As used herein, the term “protected hydroxyl” also includes groups such as OTf, that comprise good leaving groups and are useful in the synthesis of derivatives in which the OH group is replaced with an alternative group. Examples of hydroxyl protecting groups useful in the compounds of the invention include —OBn, —OTf, —OMOM, —OMEM, —OBOM, —OTBDMS, —OPMB, —OSEM.

The term “protected amino” as used herein refers to an amino group that has been protected against undesirable reactions during synthetic procedures. The protected amino group is readily converted to the free amino group, when no longer needed and/or to allow reaction of the amino group. In the compounds of the invention, the amino groups at the X position are selected from —NH₂, and —NH(C₁-C₆)alkyl. Amino protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504.

Examples of amino protecting groups useful in the compounds of the invention include, but are not limited to, acyl and acyloxy groups, for example acetyl, chloroacetyl, trichloroacetyl, o-nitrophenylacetyl, o-nitrophenoxy-acetyl, trifluoroacetyl, acetoacetyl, 4-chlorobutyryl, isobutyryl, picolinoyl, aminocaproyl, benzoyl, methoxy-carbonyl, 9-fluorenylmethoxycarbonyl, 2,2,2-trifluoroethoxycarbonyl, 2-trimethylsilylethoxy-carbonyl, tert-butyloxycarbonyl, benzyloxycarbonyl, p-nitrobenzyloxycarbonyl, 2,4-dichloro-benzyloxycarbonyl, and the like. Further examples include Nosyl (o- or p-nitrophenylsulfonyl), Bpoc (2-(4-biphenyl)isopropoxycarbonyl) and Dde (1-(4,4-dimethyl-2,6-dioxohexylidene)ethyl).

In one embodiment X is selected from the group consisting of —OH, —OBn, —OTf, —OMOM, —OMEM, —OBOM, —OTBDMS, —OPMB, —OSEM, piperazine-1-carboxylate where the N at the 4 position is substituted with (C₁-C₁₀)alkyl, —OP(O)(OH)₂, —OP(O)(OR²)₂, —NH₂, —N═C(Ph)₂, —NHZ, NH(C₁-C₁₀)alkyl and —N—Z(C₁-C₁₀)alkyl;

wherein R² is t-Bu, Bn or allyl; and Z is selected from Boc, COCF₃, Fmoc, Alloc, Cbz, Teoc and Troc.

In one embodiment, X is —OH or —NH₂.

In one embodiment X is protected or prodrug —OH.

In one embodiment X is protected —NH₂.

In one embodiment, X is selected from the group comprising —OBn, —OTf, —OMOM and -OMEM.

Methods of making compounds of the invention in which X is hydroxyl, protected hydroxyl or prodrug hydroxyl are shown in Scheme 2 below. Methods of making compounds of the invention in which X is amino or protected amino are shown in Scheme 3 below.

The compounds of the invention in which Y is a N-protecting group provide 2-methylbenzoxaxole DNA-alkylating units which may be readily attached to DNA minor groove binding units to provide highly cytotoxic duocarmycin analogues.

The term “N-protecting group” as used herein means a group that is capable of being readily removed to provide the free N and which protects the N atom against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, acyl and acyloxy groups, for example acetyl, chloroacetyl, trichloroacetyl, o-nitrophenylacetyl, o-nitrophenoxy-acetyl, trifluoroacetyl, acetoacetyl, 4-chlorobutyryl, isobutyryl, picolinoyl, aminocaproyl, benzoyl, methoxy-carbonyl, 9-fluorenylmethoxycarbonyl, 2,2,2-trifluoroethoxycarbonyl, 2-trimethylsilylethoxy-carbonyl, tert-butyloxycarbonyl, benzyloxycarbonyl, p-nitrobenzyloxycarbonyl, 2,4-dichloro-benzyloxycarbonyl, and the like. Further examples include Nosyl (o- or p-nitrophenylsulfonyl), Bpoc (2-(4-biphenyl)isopropoxycarbonyl) and Dde (1-(4,4-dimethyl-2,6-dioxohexylidene)ethyl).

In one embodiment Y is a N-protecting group selected from Boc, COCF₃, Fmoc, Alloc, Cbz, Teoc and Troc.

A person skilled in the art will be able to select protecting groups and prodrug hydroxyl moieties that are suitable for the particular synthetic scheme being used and the desired end product.

In the compounds of formula III, the DNA minor groove binding unit comprises a heteroaryl or aryl group which may be linked via an amide bond or via —NH—C(O)—CH═CH— to a second heteroaryl group or aryl group.

In other embodiments, the DNA minor groove binding unit comprises a single aryl or heteroaryl group.

In one embodiment, Ar¹, Ar² and Ar³ are independently selected from the group consisting of

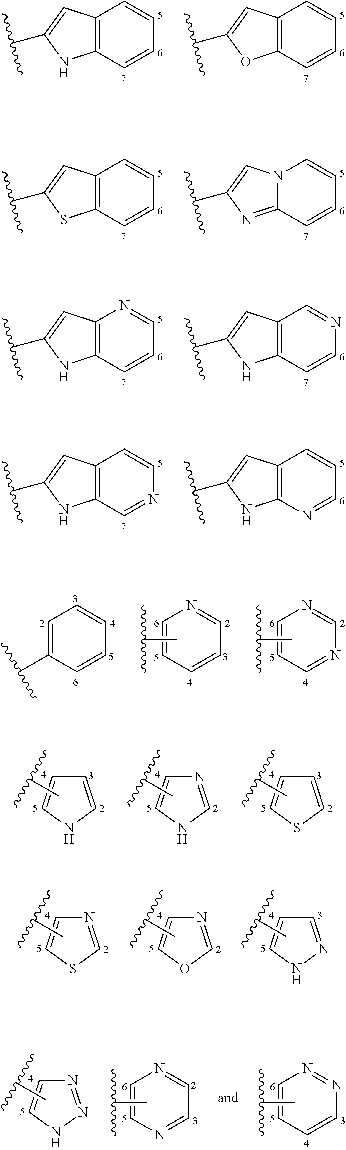

where [symbol] represents the point of attachment to the —C(O) or C(O)—CH═CH— group and each of the aryl or heteroaryl groups may be substituted at the numbered positions with up to three substituents selected from —(C₁-C₆)alkyl, —CO—(C₁-C₆)alkyl, —CONH(C₁-C₆)alkyl, —CON(C₁-C₆)alkyl(C₁-C₆)alkyl, —OH, —O—(C₁-C₆)alkyl, —NH₂, —NH(C₁-C₆)alkyl, —N(C₁-C₆)alkyl(C₁-C₆)alkyl and —NHC(O)—(C₁-C₆)alkyl.

As would be appreciated by a person skilled in the art, 5-azaindole, 6-azaindole, 7-azaindole, imidazole, thiazole, oxazole and pyrazole groups can be substituted with up to two groups and triazole, with only one.

When Ar¹ is connected to Ar² via NH—C(O) or when Ar¹ is connected to Ar³ via —NH—C(O)—CH═CH, the point of connection on Ar¹ may be any one of the numbered positions. As would be appreciated by a person skilled in the art, such a connection will reduce by one the number of possible substituents on Ar¹.

In each instance the substituents —(C₁-C₆)alkyl, —CO—(C₁-C₆)alkyl, —CONH(C₁-C₆)alkyl, —CON(C₁-C₆)alkyl(C₁-C₆)alkyl, —O—(C₁-C₆)alkyl, —NH(C₁-C₆)alkyl, —N(C₁-C₆)alkyl(C₁-C₆)alkyl and —NHC(O)—(C₁-C₆)alkyl may be independently optionally substituted with one or more of —NMe₂, —NHMe, —NH₂, —OH, morpholine and —SH.

In one embodiment, Ar¹ is a heteroaryl group. In one embodiment the heteroaryl group is an indole, azaindole, benzofuran or benzothiophene group, which is connected to the DNA alkylating unit via —C(O) or C(O)—CH═CH— at the 2-position of the heteroaryl group.

In one embodiment, Ar¹ is connected to Ar² via NH—C(O) or Ar¹ is connected to Ar³ via —NH—C(O)—CH═CH. In one embodiment, the point of connection on Ar¹ is at the 5 position of the indole, azaindole, benzofuran or benzothiophene group.

In one embodiment Ar² is selected from the group consisting of indole, azaindole, benzene, benzofuran, pyridine, pyrimidine, pyrrole, imidazole, thiophene, thiazole, oxazole, pyrazole, triazole, pyrazine or pyridazine.

In one embodiment, Ar² is selected from the group consisting of indole, azaindole, benzene, benzofuran, pyrrole or imidazole.

In one embodiment Ar³ is selected from benzene, pyridine, pyrimidine and pyridazine. In one embodiment Ar³ is benzene or pyridine, preferably benzene.

In one embodiment Y is —C(O)—Ar¹ where Ar¹ is a heteroaryl or aryl group optionally substituted with one or more of —(C₁-C₆)alkyl, —CO—(C₁-C₆)alkyl, —CONH(C₁-C₆)alkyl, —CON(C₁-C₆)alkyl(C₁-C₆)alkyl, —OH, —O—(C₁-C₆)alkyl, —NH₂, —NH(C₁-C₆)alkyl, —N(C₁-C₆)alkyl(C₁-C₆)alkyl or —NHC(O)—(C₁-C₆)alkyl,

wherein in each instance —(C₁-C₆)alkyl, —CO—(C₁-C₆)alkyl, —CONH(C₁-C₆)alkyl, —CON(C₁-C₆)alkyl(C₁-C₆)alkyl, —O—(C₁-C₆)alkyl, —NH(C₁-C₆)alkyl, —N(C₁-C₆)alkyl(C₁-C₆)alkyl and —NHC(O)—(C₁-C₆)alkyl are independently optionally substituted with one or more of —NMe₂, —NHMe, —NH₂, —OH, morpholine and —SH.

In one embodiment, Ar¹ is substituted at one position of the heteroaryl or aryl ring.

In one embodiment, Ar¹ is a heteroaryl group.

In one embodiment the heteroaryl group is an indole, azaindole, benzofuran or benzothiophene group, which is connected to the DNA alkylating unit via —C(O) or C(O)—CH═CH— at the 2-position of the heteroaryl group.

In one embodiment, Ar¹ is

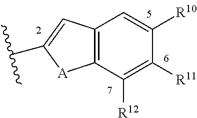

wherein A is NH, O or S, and

R¹⁰, R¹¹ and R¹² are independently selected from H, —(C₁-C₆)alkyl, —CO—(C₁-C₆)alkyl, —CONH(C₁-C₆)alkyl, —CON(C₁-C₆)alkyl(C₁-C₆)alkyl, —OH, —O—(C₁-C₆)alkyl, —NH₂, —NH(C₁-C₆)alkyl, —N(C₁-C₆)alkyl(C₁-C₆)alkyl and —NHC(O)—(C₁-C₆)alkyl,

wherein in each instance —(C₁-C₆)alkyl, —O—(C₁-C₆)alkyl, —CONH(C₁-C₆)alkyl, —N(C₁-C₆)alkyl(C₁-C₆)alkyl, and —NH—C(O)—(C₁-C₆)alkyl are independently optionally substituted with one or more of —NMe₂, —NHMe, —NH₂, —OH, morpholine and —SH.

In one embodiment, A is NH.

In one embodiment, R¹⁰, R¹¹ and R¹² are OMe.

In one embodiment, R¹⁰ is —O—(C₁-C₆)alkyl optionally substituted with one or more of —NMe₂, —NHMe, —NH₂, —OH, morpholine and —SH; R¹¹ and R¹² are both H.

In one embodiment, R¹⁰ is —NHC(O)—(C₁-C₆)alkyl optionally substituted with one or more of —NMe₂, —NHMe, —NH₂, —OH, morpholine and —SH; R¹¹ and R¹² are both H.

In one embodiment R¹⁰ is —NH—C(O)—Ar² where Ar² is an optionally substituted indole, azaindole, benzene, benzofuran, pyrrole or imidazole group; R¹¹ and R¹² are both H.

In one embodiment R¹⁰ is —NH—C(O)—Ar² where Ar² is an optionally substituted indole group; R¹¹ and R¹² are both H. In one embodiment, the indole group is substituted with —O—(C₁-C₆)alkyl optionally substituted with one or more of —NMe₂, —NHMe, —NH₂, —OH, morpholine and —SH.

In one embodiment R¹⁰ is —NH—C(O)—Ar² where Ar² is an optionally substituted benzene group; R¹¹ and R¹² are both H. In one embodiment, the benzene group is substituted with —OH, —NH₂, or —O—(C₁-C₆)alkyl, wherein —O—(C₁-C₆)alkyl is optionally substituted with —NMe₂.

In one embodiment R¹⁰ is —NH—C(O)—CH═CH—Ar³ where Ar³ is an optionally substituted benzene, pyrimidine group or pyrrole group; R¹¹ and R¹² are both H. In one embodiment, the benzene, pyrimidine group or pyrrole group is substituted with —O—(C₁-C₆)alkyl, —NH₂, or —NHC(O)—(C₁-C₆)alkyl, wherein —O—(C₁-C₆)alkyl is optionally substituted with morpholine.

In one embodiment, Ar¹ is selected from the group consisting of:

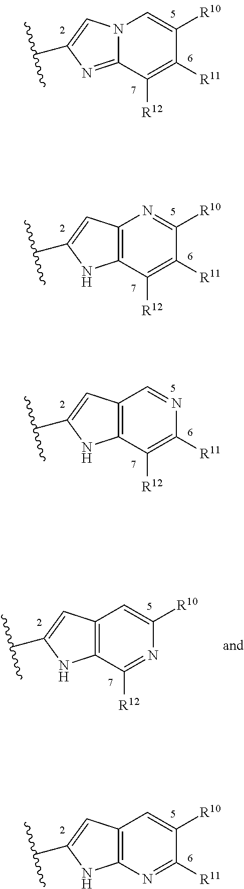

wherein R¹⁰, R¹¹ and R¹² (when present) are independently selected from H, —(C₁-C₆)alkyl, —CO—(C₁-C₆)alkyl, —CONH(C₁-C₆)alkyl, —CON(C₁-C₆)alkyl(C₁-C₆)alkyl, —OH, —O—(C₁-C₆)alkyl, —NH₂, —NH(C₁-C₆)alkyl, —N(C₁-C₆)alkyl(C₁-C₆)alkyl and —NHC(O)—(C₁-C₆)alkyl, wherein in each instance —(C₁-C₆)alkyl, —CO—(C₁-C₆)alkyl, —CONH(C₁-C₆)alkyl, —CON(C₁-C₆)alkyl(C₁-C₆)alkyl, —O—(C₁-C₆)alkyl, —NH(C₁-C₆)alkyl, —N(C₁-C₆)alkyl(C₁-C₆)alkyl and —NHC(O)—(C₁-C₆)alkyl are independently optionally substituted with one or more of —NMe₂, —NHMe, —NH₂, —OH, morpholine and —SH.

In one embodiment, R¹⁰, R¹¹ and R¹² are OMe.

In one embodiment, one of R¹⁰, R¹¹ and R¹² is —O—(C₁-C₆)alkyl optionally substituted with one or more of —NMe₂, —NHMe, —NH₂, —OH, morpholine and —SH.

In one embodiment, one of R¹⁰, R¹¹ and R¹² is —NHC(O)—(C₁-C₆)alkyl optionally substituted with one or more of —NMe₂, —NHMe, —NH₂, —OH, morpholine and —SH.

In one embodiment one of R¹⁰, R¹¹ and R¹² is —NH—C(O)—Ar² where Ar² is an optionally substituted indole, azaindole, benzene, benzofuran, pyrrole or imidazole group.

In one embodiment one of R¹⁰, R¹¹ and R¹² is —NH—C(O)—Ar² where Ar² is an optionally substituted indole group. In one embodiment, the indole group is substituted with —O—(C₁-C₆)alkyl optionally substituted with one or more of —NMe₂, —NHMe, —NH₂, —OH, morpholine and —SH.

In one embodiment one of R¹⁰, R¹¹ and R¹² is —NH—C(O)—Ar² where Ar² is an optionally substituted benzene group. In one embodiment, the benzene group is substituted with —OH, —NH₂, or —O—(C₁-C₆)alkyl, wherein —O—(C₁-C₆)alkyl is optionally substituted with —NMe₂.

In one embodiment one of R¹⁰, R¹¹ and R¹² is —NH—C(O)—CH═CH—Ar³ where Ar³ is an optionally substituted benzene, pyrimidine group or pyrrole group. In one embodiment, the benzene, pyrimidine group or pyrrole group is substituted with —O—(C₁-C₆)alkyl, —NH₂, or —NHC(O)—(C₁-C₆)alkyl, wherein —O—(C₁-C₆)alkyl is optionally substituted with morpholine.

In one embodiment Y is —C(O)—Ar¹—NH—C(O)—Ar² where Ar¹ and Ar² are heteroaryl or aryl groups optionally substituted with one or more of —(C₁-C₆)alkyl, —CO—(C₁-C₆)alkyl, —CONH(C₁-C₆)alkyl, —CON(C₁-C₆)alkyl(C₁-C₆)alkyl, —OH, —O—(C₁-C₆)alkyl, —NH₂, —NH(C₁-C₆)alkyl, —N(C₁-C₆)alkyl(C₁-C₆)alkyl or —NHC(O)—(C₁-C₆)alkyl,

wherein in each instance —(C₁-C₆)alkyl, —CO—(C₁-C₆)alkyl, —CONH(C₁-C₆)alkyl, —CON(C₁-C₆)alkyl(C₁-C₆)alkyl, —O—(C₁-C₆)alkyl, —NH(C₁-C₆)alkyl, —N(C₁-C₆)alkyl(C₁-C₆)alkyl and —NHC(O)—(C₁-C₆)alkyl are independently optionally substituted with one or more of —NMe₂, —NHMe, —NH₂, —OH, morpholine and —SH.

In one embodiment, Ar¹ is a heteroaryl group and Ar² is a heteroaryl or aryl group.

In one embodiment Ar¹ is an indole, azaindole, benzofuran or benzothiophene group, which is connected to the DNA alkylating unit at the 2-position of the heteroaryl group.

In one embodiment Ar² is selected from the group consisting of indole, azaindole, benzene, benzofuran, pyridine, pyrimidine, pyrrole, imidazole, thiophene, thiazole, oxazole, pyrazole, triazole, pyrazine or pyridazine.

In one embodiment, Ar² is selected from the group consisting of indole, azaindole, benzene, benzofuran, pyrrole or imidazole group.

In one embodiment Ar¹ is an indole, azaindole, benzofuran or benzothiophene group, which is connected to the DNA alkylating unit at the 2-position of the heteroaryl group and Ar² is selected from the group consisting of indole, azaindole, benzene, benzofuran, pyridine, pyrimidine, pyrrole, imidazole, thiophene, thiazole, oxazole, pyrazole, triazole, pyrazine or pyridazine.

In one embodiment, the point of connection on Ar¹ is at the 5 position of the indole, azaindole, benzofuran or benzothiophene group.

In one embodiment Y is —C(O)—Ar¹—NH—C(O)—CH═CH—Ar³ where Ar² and Ar³ are heteroaryl or aryl groups optionally substituted with one or more of —(C₁-C₆)alkyl, —CO—(C₁-C₆)alkyl, —CONH(C₁-C₆)alkyl, —CON(C₁-C₆)alkyl(C₁-C₆)alkyl, —OH, —O—(C₁-C₆)alkyl, —NH₂, —NH(C₁-C₆)alkyl, —N(C₁-C₆)alkyl(C₁-C₆)alkyl or —NHC(O)—(C₁-C₆)alkyl,

wherein in each instance —(C₁-C₆)alkyl, —CO—(C₁-C₆)alkyl, —CONH(C₁-C₆)alkyl, —CON(C₁-C₆)alkyl(C₁-C₆)alkyl, —O—(C₁-C₆)alkyl, —NH(C₁-C₆)alkyl, —N(C₁-C₆)alkyl(C₁-C₆)alkyl and —NHC(O)—(C₁-C₆)alkyl are independently optionally substituted with one or more of —NMe₂, —NHMe, —NH₂, —OH, morpholine and —SH.

In one embodiment, Ar¹ is a heteroaryl group and Ar³ is a heteroaryl or aryl group.

In one embodiment Ar¹ is an indole, azaindole, benzofuran or benzothiophene group, which is connected to the DNA alkylating unit at the 2-position of the heteroaryl group.

In one embodiment Ar³ is selected from benzene, pyridine, pyrimidine and pyridazine. In one embodiment the Ar³ is benzene or pyridine, preferably benzene.

In one embodiment Ar¹ is an indole, azaindole, benzofuran or benzothiophene group, which is connected to the DNA alkylating unit at the 2-position of the heteroaryl group and Ar³ is selected from benzene, pyridine, pyrimidine and pyridazine.

In one embodiment, the point of connection on Ar¹ is at the 5 position of the indole, azaindole, benzofuran or benzothiophene group.

In one embodiment Y is —C(O)—CH═CH—Ar³ where Ar³ is a heteroaryl or aryl group optionally substituted with one or more of —(C₁-C₆)alkyl, —CO—(C₁-C₆)alkyl, —CONH(C₁-C₆)alkyl, —CON(C₁-C₆)alkyl(C₁-C₆)alkyl, —OH, —O—(C₁-C₆)alkyl, —NH₂, —NH(C₁-C₆)alkyl, —N(C₁-C₆)alkyl(C₁-C₆)alkyl or —NHC(O)—(C₁-C₆)alkyl,

wherein in each instance —(C₁-C₆)alkyl, —CO—(C₁-C₆)alkyl, —CONH(C₁-C₆)alkyl, —CON(C₁-C₆)alkyl(C₁-C₆)alkyl, —O—(C₁-C₆)alkyl, —NH(C₁-C₆)alkyl, —N(C₁-C₆)alkyl(C₁-C₆)alkyl and —NHC(O)—(C₁-C₆)alkyl are independently optionally substituted with one or more of —NMe₂, —NHMe, —NH₂, —OH, morpholine and —SH.

In one embodiment Ar³ is selected from benzene, pyridine, pyrimidine and pyridazine. In one embodiment the Ar³ is benzene or pyridine, preferably benzene.

In one embodiment Ar³ is a benzene group optionally substituted with —O—(C₁-C₆)alkyl which is optionally substituted with one or more of —NMe₂, —NHMe, —NH₂, —OH, morpholine and —SH.

The compounds of formula I, II and III are seco precursors to compounds of formula IV, which are thought to be the active agents in vivo.

In a fourth aspect the invention provides a compound of formula IV or a pharmaceutically acceptable salt, hydrate or solvate thereof

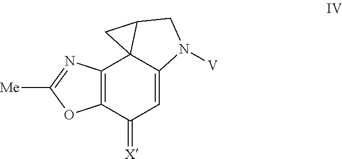

wherein V is Y or DB and X, Y and DB have the same meaning as defined for compounds of formulae I, II, and III and X′ is X with loss of H.

Compounds of formula IV may be formed through in vitro, or in vivo rearrangement and concomitant elimination of H-LG from the corresponding seco-compound of formula I, II and III. All embodiments of the invention described herein for a compound of formulae I, II or III, or any of their moieties thereof are also specifically contemplated as part of the aspect of the invention that relates to compounds of formula IV, unless context dictates otherwise.

Compounds of formulae I, II and III contain leaving groups (LG) which facilitate cyclopropyl ring formation under physiological conditions to form compounds of formula IV.

In one aspect, the invention provides a compound selected from the group consisting of any one of compounds 49, 18, 50, 51, 23, 52, 53, 57, 58, 59, 63, 64, 65, 66, 70, 74, 79, 80, 81, 82, 83, 24, 26, 85, 87, 88, 89, 90, 91 and 93.

In one embodiment, the invention provides a compound selected from the group consisting of any one of compounds 49, 18, 50, 51, 23, 52, 57, 53, 58, 59, 63, 64, 65, 66, 70, 74, 79, 80, 81, 82, 83, 24, 85, 88 and 90.

In one embodiment, the invention provides a compound selected from the group consisting of:

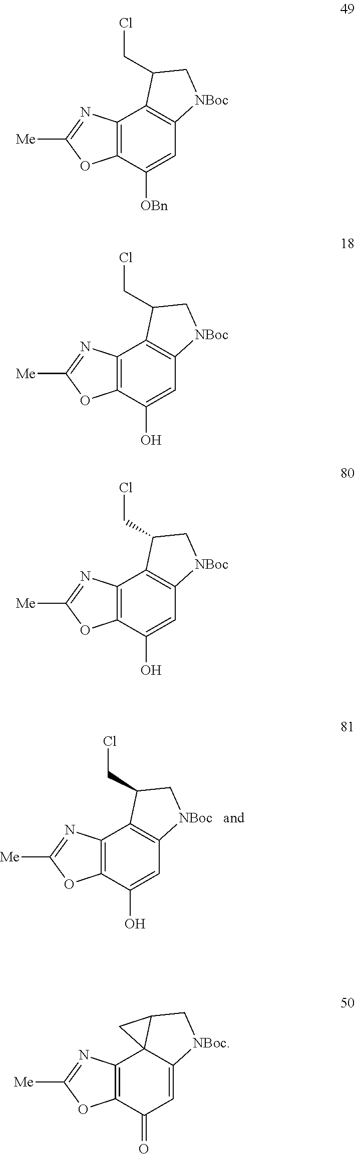 [SMILES: Cc1nc2c3c(cc(OCc4ccccc4)c2o1)N(C(=O)OC(C)(C)C)CC3CCl ; Cc1nc2c3c(cc(O)c2o1)N(C(=O)OC(C)(C)C)CC3CCl ; Cc1nc2c3c(cc(O)c2o1)N(C(=O)OC(C)(C)C)C[C@H]3CCl ; Cc1nc2c3c(cc(O)c2o1)N(C(=O)OC(C)(C)C)C[C@@H]3CCl ; Cc1nc2c(o1)C(=O)C=C1N(C(=O)OC(C)(C)C)CC3CC123]

In one embodiment, the invention provides a compound selected from the group consisting of:

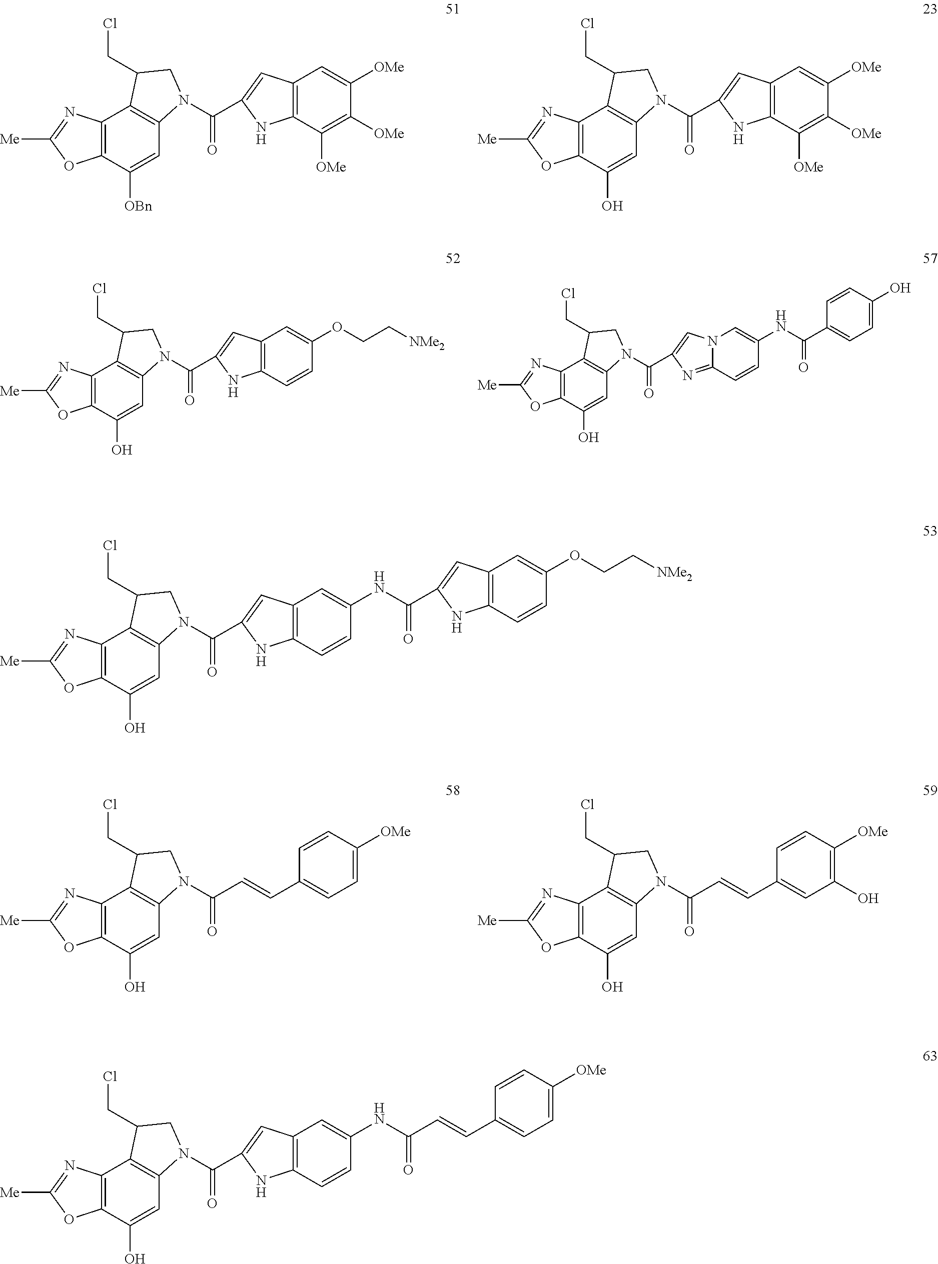

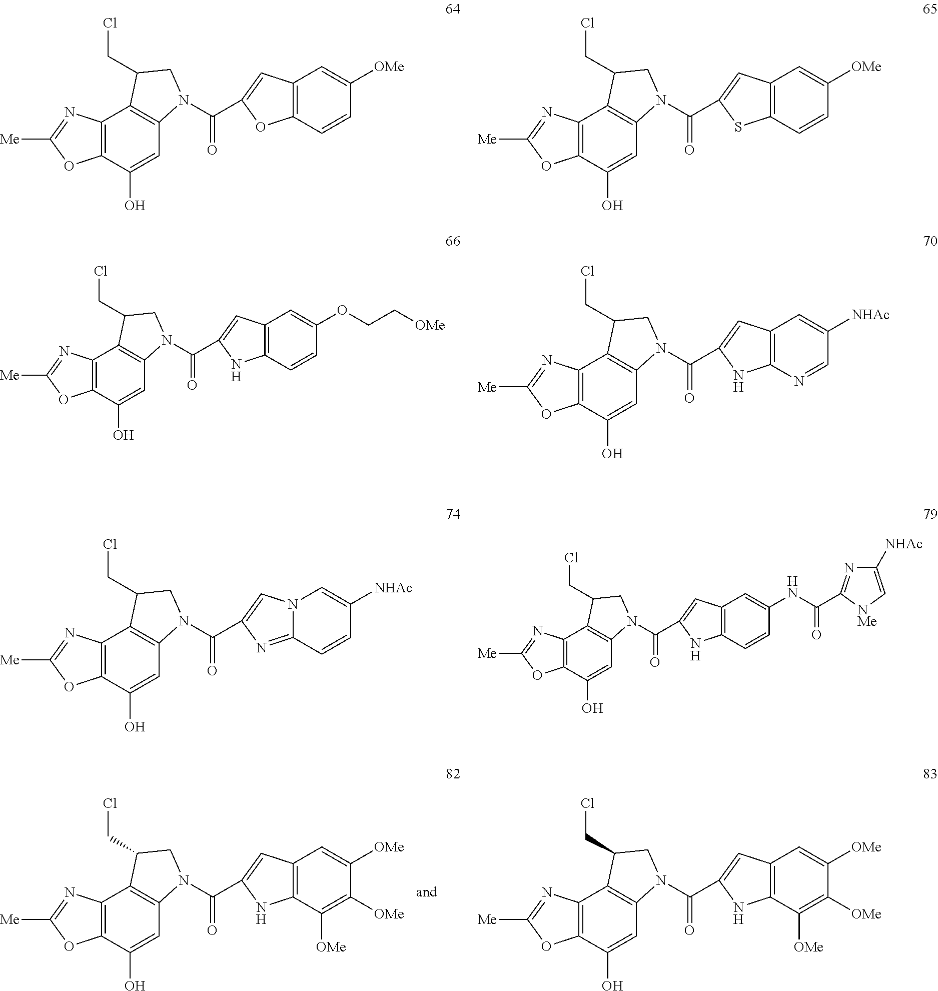

In one embodiment, the invention provides a compound selected from the group consisting of:

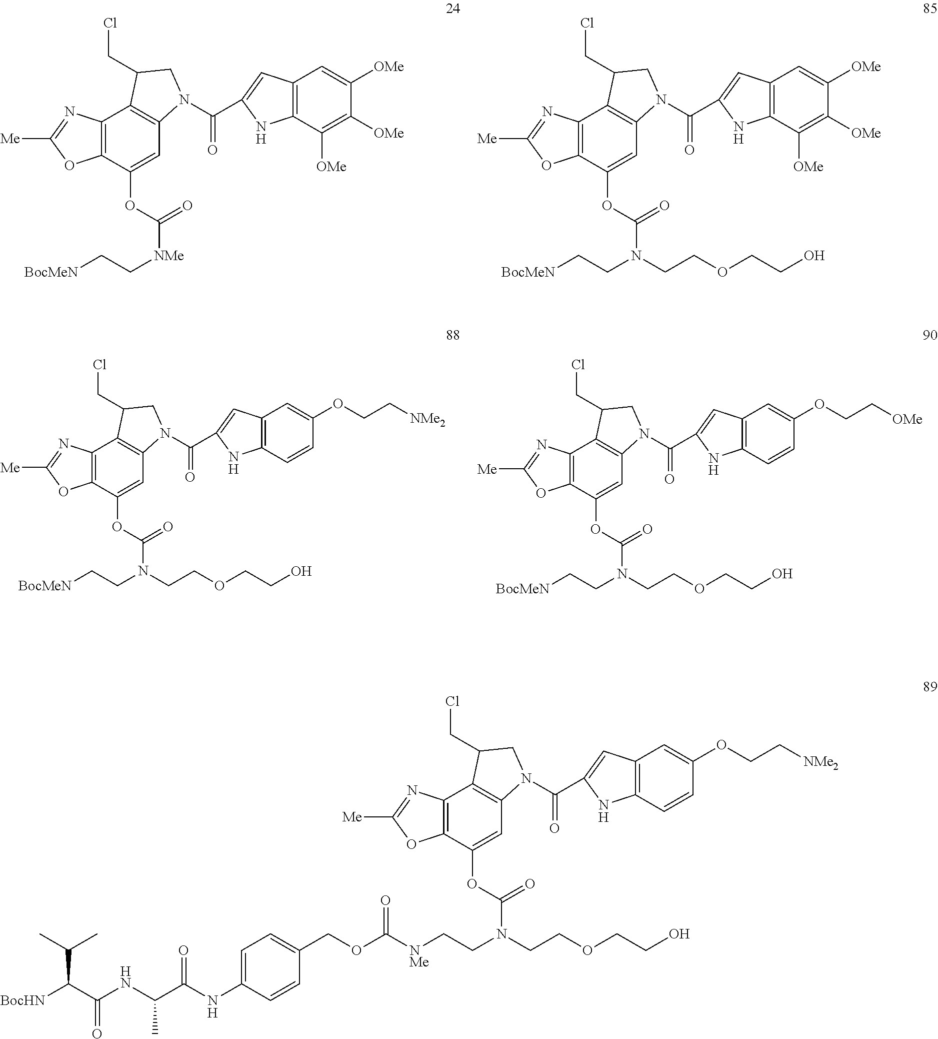

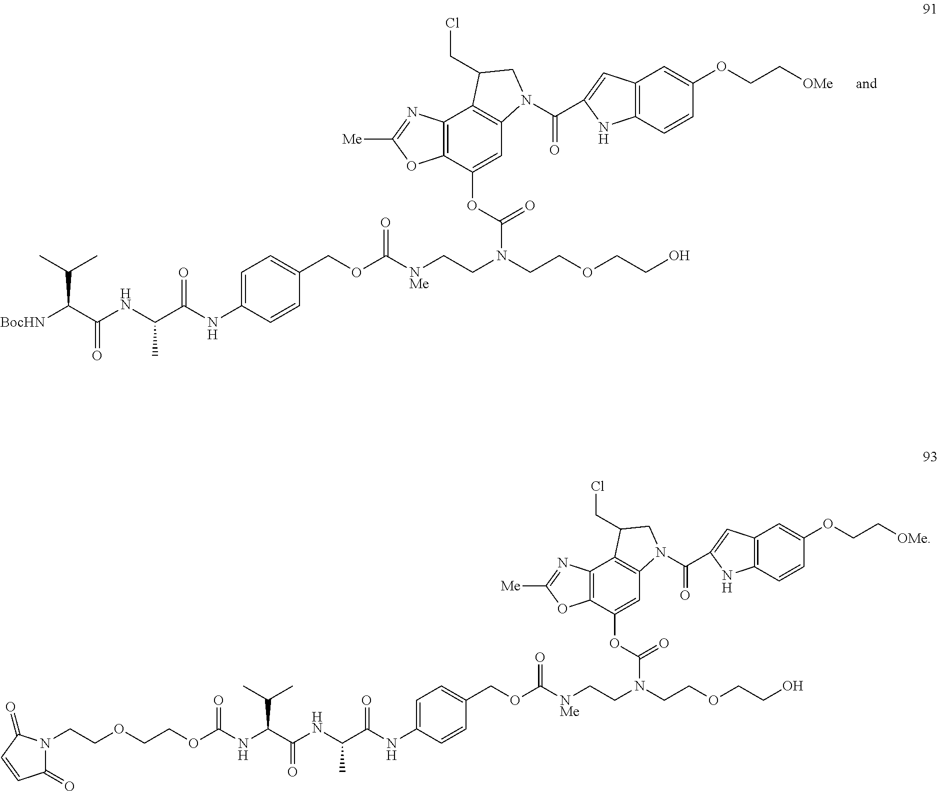 [SMILES: COCCOc1ccc2[nH]c(C(=O)N3CC(CCl)c4c3cc(OC(=O)N(CCOCCO)CCN(C)C(=O)OCc3ccc(NC(=O)[C@H](C)NC(=O)[C@@H](NC(=O)OC(C)(C)C)C(C)C)cc3)c3oc(C)nc43)cc2c1 ; COCCOc1ccc2[nH]c(C(=O)N3CC(CCl)c4c3cc(OC(=O)N(CCOCCO)CCN(C)C(=O)OCc3ccc(NC(=O)[C@H](C)NC(=O)[C@@H](NC(=O)OCCOCCN5C(=O)C=CC5=O)C(C)C)cc3)c3oc(C)nc43)cc2c1]

### 5.3 Synthesis of Compounds of the Invention

The compounds of the invention may be prepared using the methods and procedures described herein or methods and procedures analogous thereto. Other suitable methods for preparing compounds of the invention will be apparent to those skilled in the art.

It will be appreciated that where typical or preferred process conditions (for example, reaction temperatures, times, mole ratios of reactants, solvents, pressures, etc.) are indicated, other process conditions can also be used unless otherwise stated. Optimum reaction conditions may vary with the particular reactants used.

The starting materials useful in the methods and reactions are commercially available or can be prepared by known procedures or modifications thereof, for example those described in standard reference texts such as Fieser and Fieser's Reagents for Organic Synthesis, Volumes 1-15 (John Wiley and Sons, 1991), Organic Reactions, Volumes 1-40 (John Wilev and Sons. 1991). March's Advanced Organic Chemistry. (John Wiley and Sons, 4th Edition), and Larock's Comprehensive Organic Transformations (VCH Publishers Inc., 1989).

The various starting materials, intermediates, and compounds may be isolated and purified where appropriate using conventional techniques such as precipitation, filtration, crystallization, evaporation, distillation, and chromatography. Characterization of the compounds may be performed using conventional methods such as by melting point, mass spectrum, nuclear magnetic resonance, and various other spectroscopic analyses.

Conventional protecting groups may be necessary to prevent certain functional groups from undergoing undesired reactions. The need for protection and deprotection and the selection of appropriate protecting groups can be readily determined by a person skilled in the art. Suitable protecting groups for various functional groups as well as suitable conditions for protecting and deprotecting particular functional groups are well known in the art (see, for example, T. W. Greene and G. M. Wuts, Protecting Groups in Organic Synthesis, Third Edition, Wiley, New York, 1999).

The individual enantiomers of payloads containing the 2-methylbenzoxazole alkylating subunit can be prepared using chiral HPLC resolution of suitable intermediates, for example, compounds 49 or 18. Suitable columns include those used to resolve related alkylating subunits e.g. CBI (J. Am. Chem. Soc. (1994) 116, 7996), CTI (Bioorg. Med. Chem. Lett. (2009) 19, 6962), iso-DSA (J. Am. Chem. Soc. (2009) 131, 1187), CImI (Bioorg. Med. Chem. (2016) 24, 4779). The resolved intermediates can be converted to payloads and drug-linker conjugates by methods analogous to those described for the racemates.

General methods to make the 2-methylbenzoxazole alkylating subunit where X=protected OH are shown in Scheme 2. Compounds 5 and 7 in this scheme are easily converted to 2-methylbenzoxazole alkylating subunits where X=free OH and X=prodrug OH.

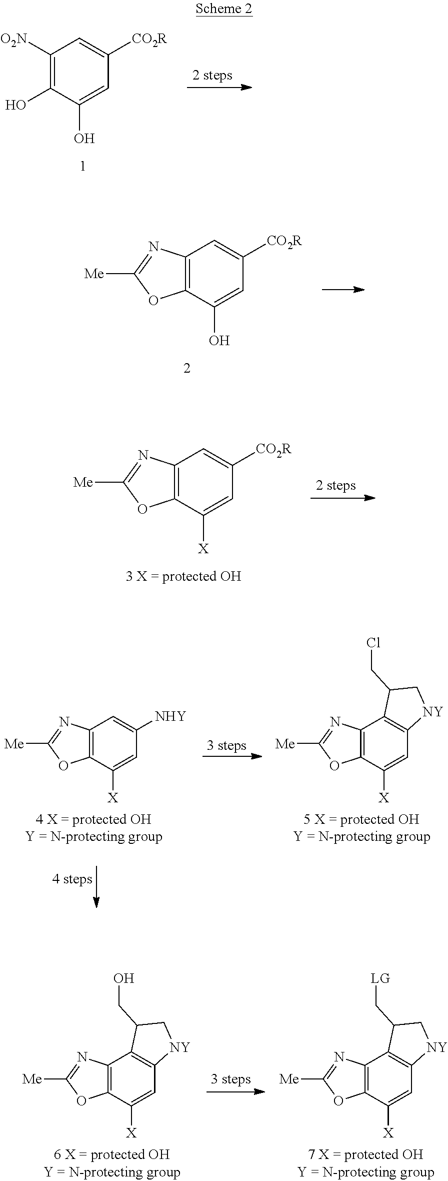

In this scheme 3,4-dihydroxy-5-nitrobenzoate esters 1 serve as starting materials. These may be prepared, for example, by oxidation of 3,4-dihydroxy-5-nitrobenzaldehyde to the corresponding acid, followed by esterification with an alcohol ROH. Alternatively, 1 may be prepared by nitration of 4-hydroxy-3-methoxybenzoic acid, followed by dealkylation of the methoxy group using a reagent such as HBr or BBr₃, followed by esterification with an alcohol ROH.

The nitro group of 1 is reduced to the corresponding amine by exposure to suitable conditions (e.g. hydrogenation over a Pd or Pt catalyst, or exposure to Fe(III) salts under acidic conditions) and the product is treated with a trialkylorthoacetate, such as trimethylorthoacetate, under acidic conditions to induce cyclisation to the substituted 2-methylbenzoxazole 2.

At this point in the synthesis, various X=protected —OH can be introduced. For example, a benzyl protecting group can be introduced by reaction of 2 with benzyl bromide or benzyl chloride under basic conditions to give 3 where X=OBn. Similarly a MOM protecting group can be introduced by reaction with MOMCl; a MEM protecting group can be introduced by reaction with MEMCl; a BOM protecting group can be introduced by reaction with BOMCI; a TBDMS protecting group can be introduced by reaction with TBDMSCl; a PMB protecting group can be introduced by reaction with PMBCl; and a SEM protecting group can be introduced by reaction with SEMCl. For the different protecting groups different reaction conditions (e.g. base, solvent, temperature, chloride or bromide reagent, additive such as an iodide salt) may be chosen as appropriate to optimise the yield of 3.

The ester of 3 is then hydrolysed under standard conditions to give the corresponding acid, which is converted to the NHY group of 4. Where Y represents a carbamate protecting group the second of these steps may be conveniently conducted in a single pot using the diphenylphosphoryl azide (DPPA) reagent in combination with an organic base such as trimethylamine and a suitable alcohol. For example, DPPA and t-BuOH will give 4 where Y=Boc. Similarly the use of benzyl alcohol will give Y=Cbz, the use of 2-(trimethylsilyl)ethanol will give Y=Teoc, and the use of 2,2,2-(trichloro)ethanol will give Y=Troc. Alternatively, the intermediate isocyanate may be reacted with water instead of an alcohol to generate the corresponding unprotected amine. This may then be converted to the protected form under standard conditions e.g. reaction with trifluoroacetic anhydride will give Y═COCF₃, while reaction with benzyl chloroformate will give Y=Cbz.

Compound 4 can be halogenated selectively in the 4-position by reaction with a suitable reagent, e.g. NBS introduces bromide at the 4-position while NIS introduces iodide at the 4-position. These reactions are best performed with a single equivalent of halogenating agent to minimise dihalogenation at the 4- and 6-positions. To make the alkylating subunit where the leaving group is chloride, i.e. 5, the halogenated intermediate is then reacted with 1,3-dichloropropene in the presence of a suitable base such as NaH or K₂CO₃, which serves to deprotonate the NHY group and direct chloroallylation to this position. This intermediate is then treated with a suitable reagent, such as tributyltin hydride, or tris(trimethylsilyl)silane, to abstract the halogen atom and initiate a radical mediated-cyclisation onto the pendant chloroallyl group, which generates product 5.

Alkylating subunits where the leaving group is a halide or sulfonate can be made via intermediate 6. Compound 6 can be prepared from 4 by a modification of the steps described above. Allyl bromide is used in place of 1,3-dichloropropene, and the radical-mediated cyclisation is conducted in the presence of the spin trap TEMPO (2,2,6,6-tetramethyl-1-piperidinyloxy). This generates a product containing a N—O bond which is cleaved by exposure to Zn and an acid such as acetic acid, to generate 6. The primary alcohol of 6 is converted to LG=halide or sulfonate by the use of standard reagents, e.g. bromine and triphenylphosphine can be used to make LG=Br, while methanesulfonyl chloride and triethylamine can be used to make LG=OSO₂Me.

The protecting groups of X and of Y can be selectively removed and replaced at the stage of compounds 5 and 7. For several of the Y protecting groups which may be less stable to the conditions used in the synthetic steps after compound 4, this is likely to be the preferred method for their introduction. For example, Y═COCF₃ may be unstable to the basic conditions used in the allylation reaction but can be introduced by deprotection of 5 or 7 (where Y=Boc for example) followed by reaction with trifluoroacetic anhydride. Similarly, Y=Fmoc may also be unstable to the same basic conditions, while Y=Alloc may interfere in the radical-mediated cyclisation. Nevertheless, these alternative protecting groups can be very useful in subsequent reactions of 5 and 7 where stability to different reaction conditions is required. This is illustrated below, as an example, in the synthesis of alkylating subunits where X=protected NH₂.

For 5 and 7 the group X=protected OH can also be converted to free OH (i.e. compound 8 below), and thereby to prodrug OH. The latter conversion proceeds via well-established synthetic methods. For example, reaction of 8 with a 4-alkylpiperazinecarbonyl chloride gives 5 and 7 in which X=piperazine-1-carboxylate where the N at the 4-position is substituted with an alkyl group. In another example the free OH of 8 is reacted with a phosphoramidite reagent of the general structure R₂NP(OR²)₂, where R is lower alkyl e.g. ethyl or isopropyl, and R⁴ is t-Bu, Bn or allyl. The intermediate product is then oxidised with a suitable reagent such as hydrogen peroxide or an organic peracid to give 5 and 7 where X═OP(O)(OR²)₂.

General methods to make the 2-methylbenzoxazole alkylating subunit where X═NH₂, protected NH₂, NHR¹, or protected NHR¹ where R¹ is (C₁-C₆) alkyl, are shown in Scheme 3.

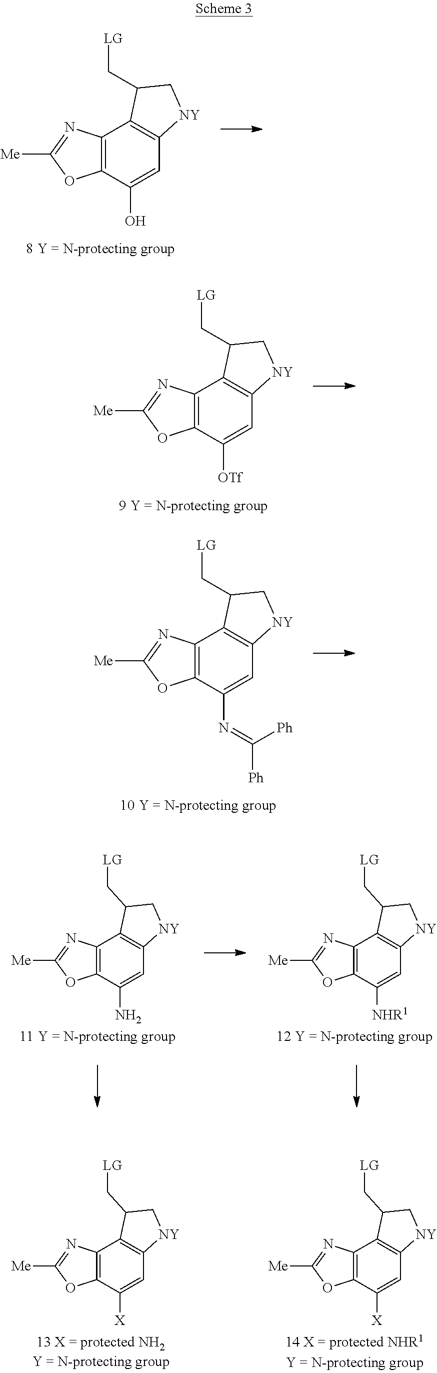

Compound 8 containing a free OH group is reacted with triflic anhydride and a suitable base, such as triethylamine, to give compound 9. This compound is reacted with benzophenone imine using a suitable catalyst such as Pd(OAc)₂ and ligand such as BINAP to effect amination and generate compound 10. The benzophenone imine is cleaved under acidic conditions to generate compound 11. The acidic conditions required mean that some protecting groups Y are more suitable than others. For example, Y═COCF₃ is more suitable than Y=Boc, therefore one convenient route entails swapping Y=Boc to Y═COCF₃ at the stage of compound 7. Other combinations of protecting groups are also possible and can offer different synthetic advantages depending on the particular target compounds.

Compound 11 can be further converted to alkylating subunits 12 bearing an NHR¹ (where R¹ is (C₁-C₆) alkyl) substituent at the X position. For example 11 can be reacted with acetic formic anhydride to generate a formylated intermediate that is reduced using borane to give 12 where R¹=Me. Alternatively 11 can be condensed with aldehydes R¹CHO where R¹ is (C₁-C₆)alkyl to give imines which are reduced with reagents such as sodium borohydride or sodium cyanoborohydride to give 12 where R¹═(C₁-C₆)alkyl. Compounds 11 and 12 can be converted to protected analogues 13 and 14 using standard conditions as indicated above, for example reaction with trifluoroacetic anhydride will generate X═NHZ and NZR¹ where Z═COCF₃ and R¹ is (C₁-C₆) alkyl, while reaction with fluorenylmethyloxycarbonyl chloride will generate X═NHZ and NZR¹ where Z=Fmoc and R¹ is (C₁-C₆) alkyl.

General methods to make DNA alkylating agents incorporating the 2-methylbenzoxazole alkylating subunit and a DNA minor groove binding moiety (DB) are shown in Scheme 4.

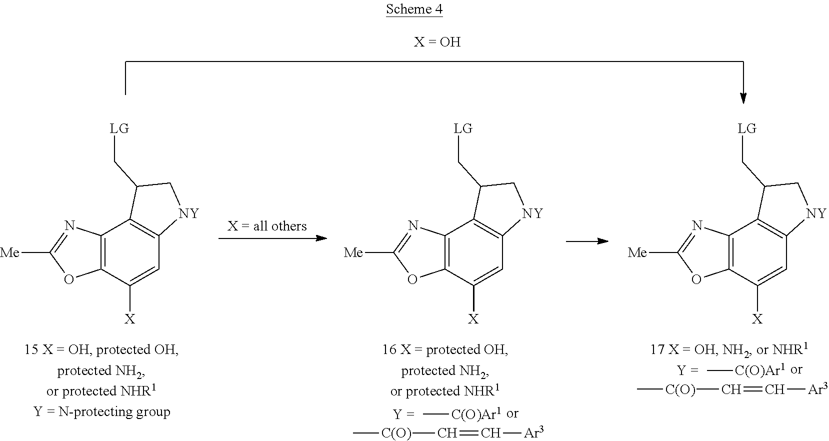

In cases where 15 contains X═OH the protecting group Y is removed by an appropriate method (e.g. for Y=Boc by treatment with HCl) and the resulting intermediate is reacted with the DNA minor groove binding unit Ar¹CO₂H or Ar³CH═CHCO₂H in the presence of a suitable amide coupling reagent such as EDCI or HOBt. Other activated forms of the DNA minor groove binding unit may also be used, such as acid chlorides. This approach directly generates 17 where X═OH.

In the cases where 15 contains X═protected OH or protected NH₂ or protected NHR¹ the synthesis proceeds in 2 steps, via intermediate 16. It will be clear to a person skilled in the art that appropriate choices of the protecting groups in X and Y should be made in order to allow deprotection of Y in the presence of the protecting group used in X. For example, if Y is Boc then X should not be NHBoc or NRBoc, or any other protecting group which is too acid-sensitive e.g. NHTeoc or NRTeoc. Suitable combinations of orthogonal protecting groups are readily available by following the general schemes shown above. The amide coupling to form compound 16 where Y═—C(O)Ar¹ or —C(O)—CH═CH—Ar³ is performed in the same general way as described above, and 16 is converted to 17 by removing the protecting group in X. In the case where compound 17 contains X═NH₂ then a benzophenone imine may serve as a suitable protected precursor, i.e. 15 where X ═N═C(Ph)₂.

The same approaches can be used to make payloads in which the side chain contains two linked aromatic rings, by substituting the appropriate side chain acids i.e. by using Ar²C(O)NHAr¹CO₂H or Ar³CH═CHC(O)NHAr¹CO₂H.

Scheme 5 shows a prophetic example of the synthesis of a compound of the invention in which X is NH₂.

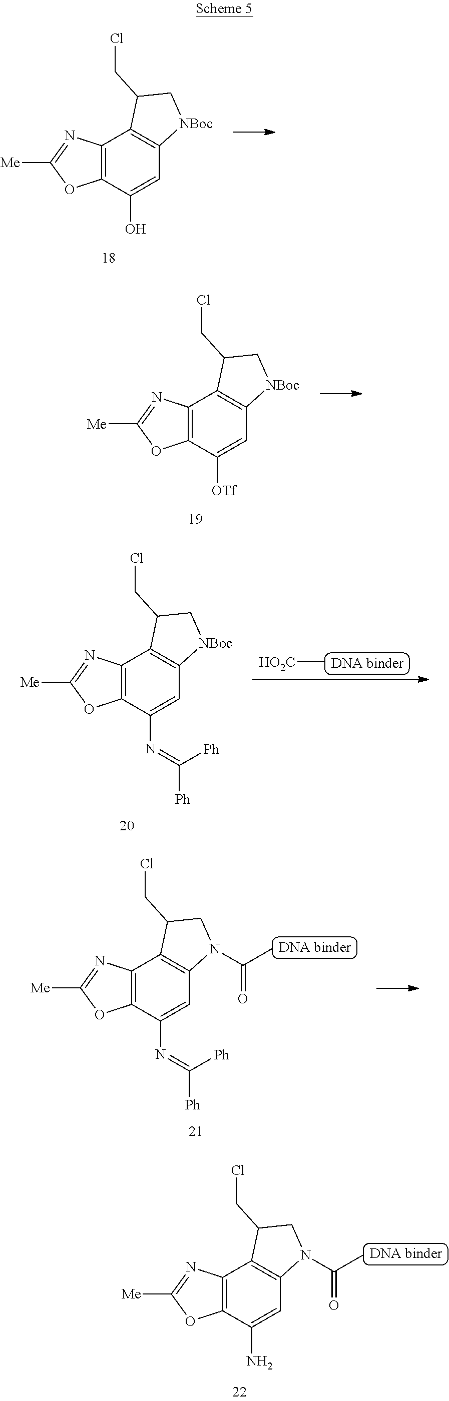

In the synthesis proposed in Scheme 5, 2-methylbenzoxazole alkylating subunits containing a free amino group (general structure 22) can be prepared by a method analogous to the reported method for seco-CBI compounds (Bioorg. Med. Chem. (2016) 24, 6075). In particular, compound 18 is converted to the triflate 19 using triflic anhydride and triethylamine. Compound 19 is aminated using benzophenone imine and a Pd(OAc)₂ catalyst and a BINAP ligand to give 20. The Boc protecting group is selectively cleaved using HCl in methanol or TFA in dichloromethane, and the resulting intermediate is reacted with a suitable side chain acid, using EDCI as a coupling reagent, to give amide 21. The benzophenone protecting group is removed using aqueous acetic acid to give the desired product 22. This method can also be applied to the R and S enantiomers of 18 to give the corresponding R and S enantiomers of 22.

Many of the DNA minor groove binding unit acids Ar¹CO₂H or Ar³CH═CHCO₂H with suitable substituents are commercially available compounds, or can be readily made from commercially available compounds by simple functional group changes e.g. esters and nitriles can be hydrolysed to carboxylic acids, nitro substituents can be reduced to amino substituents, which can be further alkylated or acylated, and halide substituents can undergo a range of metal-mediated reactions and/or displacement reactions to access a variety of desired substituents. In addition, numerous other aryl and heteroaryl compounds useful for preparing suitable DNA minor groove binding unit acids are known compounds, or can be made by modifications of known synthetic methods. Many of these methods are described in “Comprehensive Heterocyclic Chemistry III”, edited by A. R. Katritzky, C. A. Ramsden, E. F. V. Scriven and R. J. K. Taylor, Elsevier, 2008. For example, the synthesis of indoles is reviewed in section 3.03 (Vol 3, pp 269-351), the synthesis of azaindoles is reviewed in section 10.06 (Vol 10, pp 263-338), the synthesis of benzofurans is reviewed in section 3.07 (Vol 3, pp 497-569), and the synthesis of benzothiophenes is reviewed in section 3.11 (Vol 3, pp 834-930), all of which are incorporated by reference herein. There are many other reviews on the synthesis of substituted heteroaryl compounds, for example indole synthesis is reviewed in the following publications: Chem. Rev. (2006) 106, 2875; Chem. Rev. (2005) 105, 2873; Perkin Trans 1 (2000), 1045.

More specifically, for application as DNA minor groove binding unit acids, methods are known that produce heteroaryl compounds with a carboxylate substituent in the desired 2-position. Many of these are included in the reviews cited above, but particular examples are also given here: for indole-2-carboxylates Org. Lett. (2016) 18, 3586; Org. Lett. (2004) 6, 2953; for azaindole-2-carboxylates WO 2009/030725; for benzofuran-2-carboxylates Org. Lett. (2013) 15, 3876; J. Chem. Soc., Perkin Trans. 2 (1998) 1063; and for benzothiophene-2-carboxylates Org. Lett. (2013) 15, 3876; Org. Lett. (2012) 14, 5334.

Many of these methods have been adopted in the synthesis of particular DNA minor groove binding unit acids for use in the preparation of duocarmycin analogues. For example, the synthesis of 72 different substituted indole-2-carboxylates and their coupling to the seco-CBI alkylating subunit is described in Bioorg. Med. Chem. (2003) 11, 3815. Other examples of a range of substituted indole-2-carboxylates used to prepare duocarmycin analogues are described in J. Med. Chem. (2017) 60, 5834; Chem Med Chem (2014) 9, 2193; Eur. J. Org. Chem. (2006) 2314; and J. Am. Chem. Soc. (1997) 119, 4977; while the similar use of a variety of substituted cinnamic acids in described in J. Med. Chem. (1997) 40, 972. The synthesis of DNA minor groove binding unit acids of the type in which Ar¹ is connected to an aryl or heteroaryl group Ar³ has also been reported, for example in WO 2011/133039; J. Med. Chem. (2003) 46, 634; and J. Org. Chem. (2001) 66, 6654 (which describes the synthesis of 132 such side chains combining, amongst other ring systems, indoles, benzofurans, benzothiophenes, pyrroles, thiophenes, imidazoles, and thiazoles).

A person skilled in the art will recognise that many other side chains in which Ar¹ is connected to Ar² or Ar³ can be prepared by forming an amide bond between an Ar¹ unit containing an amino substituent and an Ar² or Ar³ unit containing a carboxylic acid substituent, using standard methods and coupling reagents, and using appropriate protecting groups as appropriate for the other substituents involved in the particular examples. Therefore, there is a wealth of information and precedent describing general and specific methods of preparation of the acids Ar¹CO₂H or Ar³CH═CHCO₂H or Ar²CONHAr¹CO₂H or Ar³CH═CHCONHAr¹CO₂H to be reacted with the DNA alkylating unit, in the synthesis of the compounds of the invention.

DNA alkylating agents incorporating the 2-methylbenzoxazole alkylating subunit and a minor groove binding side chain can be used as payloads for ADCs. For this purpose, a linker must be used to connect the payload to the antibody. The linker may be connected to the payload in several different ways. For example, the linker can be connected at the X position, which may be via a carbamate (—OC(O)NHR or —OC(O)NR₂ or —NHC(O)OR or —NRC(O)OR′) or via an ether (—OR) functional group. These types of linkers need to fragment to release X═OH or NH₂ or NHR after the ADC is metabolised, a type of connection known as ‘traceless’ linkers. Several examples of suitable linker types are known, which often incorporate self-immolative spacers such as para-aminobenzylethers or para-aminobenzylcarbamates, which may be further substituted in a way that affects their fragmentation rate, or connected to an extra cleavable spacer such as an N,N-dialkyl-1,2-diaminoethane.

Illustrative examples are shown below of the syntheses of representative drug-linker compounds containing traceless linkers to the X position.

Scheme 6 outlines preparation of a compound of the invention where a linker is connected via a phenol carbamate.

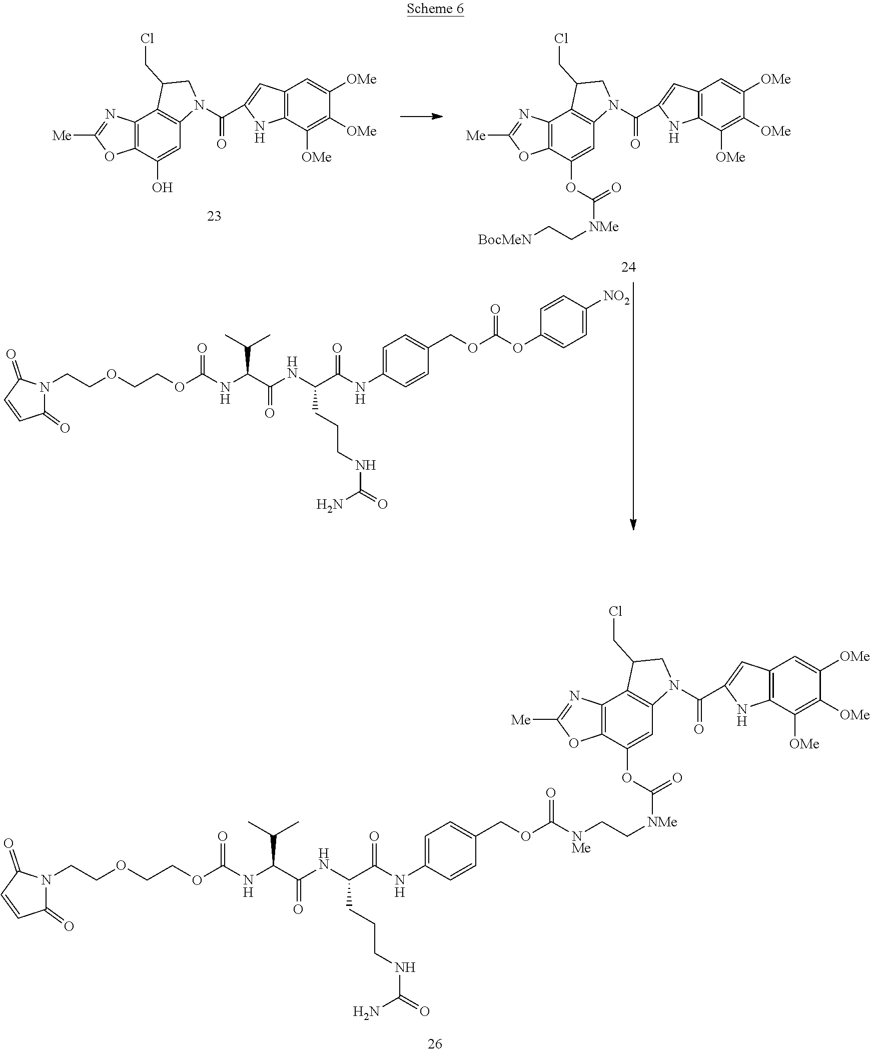

In this scheme compound 23 is reacted with 4-nitrophenyl chloroformate and the product is then reacted with monoBoc-protected N,N′-dimethyl-1,2-diaminoethane to give 24.

The Boc group of 24 is deprotected using TFA to give the corresponding TFA salt. The activated maleimide-valine-citrulline-PABA compound 25 is prepared as described (Mol. Pharm. (2015) 12, 1813). The TFA salt and 25 are reacted under slightly basic conditions giving drug-linker 26.

Scheme 7 outlines preparation of a compound of the invention where a linker is connected via a phenol ether.

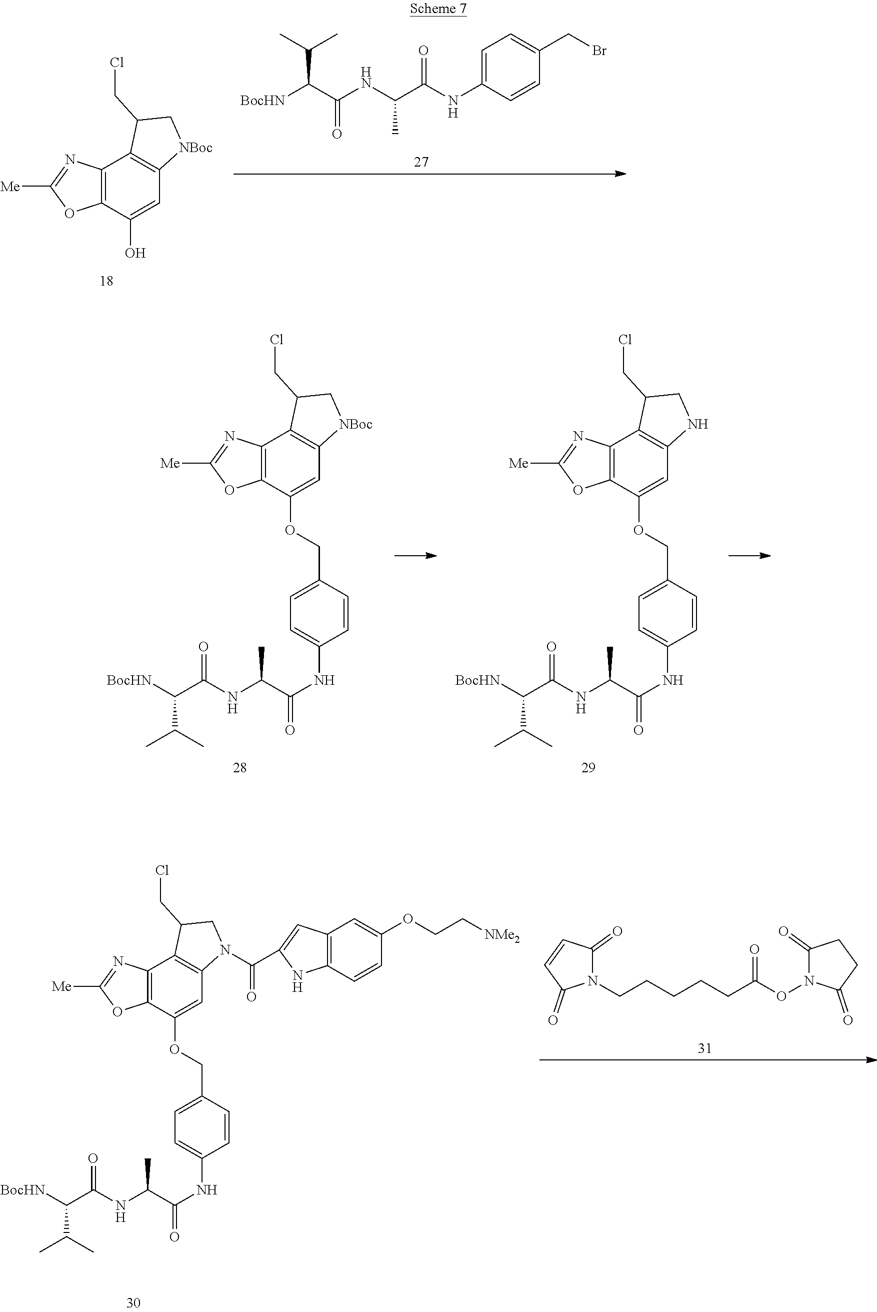 [SMILES: Cc1nc2c3c(cc(O)c2o1)N(C(=O)OC(C)(C)C)CC3CCl ; CC(C)[C@H](NC(=O)OC(C)(C)C)C(=O)N[C@@H](C)C(=O)Nc1ccc(CBr)cc1 ; Cc1nc2c3c(cc(OCc4ccc(NC(=O)[C@H](C)NC(=O)[C@@H](NC(=O)OC(C)(C)C)C(C)C)cc4)c2o1)N(C(=O)OC(C)(C)C)CC3CCl ; Cc1nc2c3c(cc(OCc4ccc(NC(=O)[C@H](C)NC(=O)[C@@H](NC(=O)OC(C)(C)C)C(C)C)cc4)c2o1)NCC3CCl ; Cc1nc2c3c(cc(OCc4ccc(NC(=O)[C@H](C)NC(=O)[C@@H](NC(=O)OC(C)(C)C)C(C)C)cc4)c2o1)N(C(=O)c1cc2cc(OCCN(C)C)ccc2[nH]1)CC3CCl ; O=C(CCCCCN1C(=O)C=CC1=O)ON1C(=O)CCC1=O]

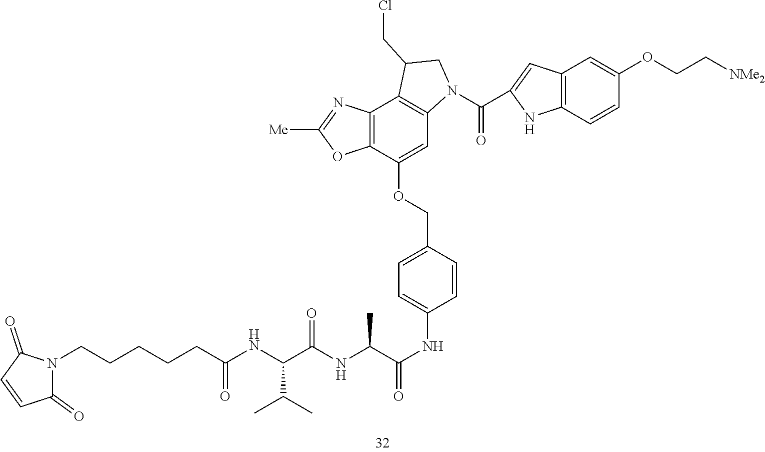 [SMILES: Cc1nc2c3c(cc(OCc4ccc(NC(=O)[C@H](C)NC(=O)[C@@H](NC(=O)CCCCCN5C(=O)C=CC5=O)C(C)C)cc4)c2o1)N(C(=O)c1cc2cc(OCCN(C)C)ccc2[nH]1)CC3CCl]

In this scheme, phenol 18 is deprotonated using a suitable base such as MeLi and then reacted with the known valine-alanine-PAB-bromide compound 27 (WO 2018/035391) to give compound 28. Both Boc protecting groups are removed by treating with TFA and the Boc protecting group on the aliphatic amine is replaced by subsequently treating with (Boc)₂O in the presence of DIPEA to give compound 29. This compound is reacted with 5-(2-(dimethylamino)ethoxy)-1H-indole-2-carboxylic acid using EDCI as a coupling reagent to generate 30. The Boc protecting group is removed with TFA and the amine is reacted with the commercially available NHS ester 31 giving payload-linker compound 32.

Scheme 8 outlines preparation of a compound of the invention where a linker is connected via an amino carbamate.

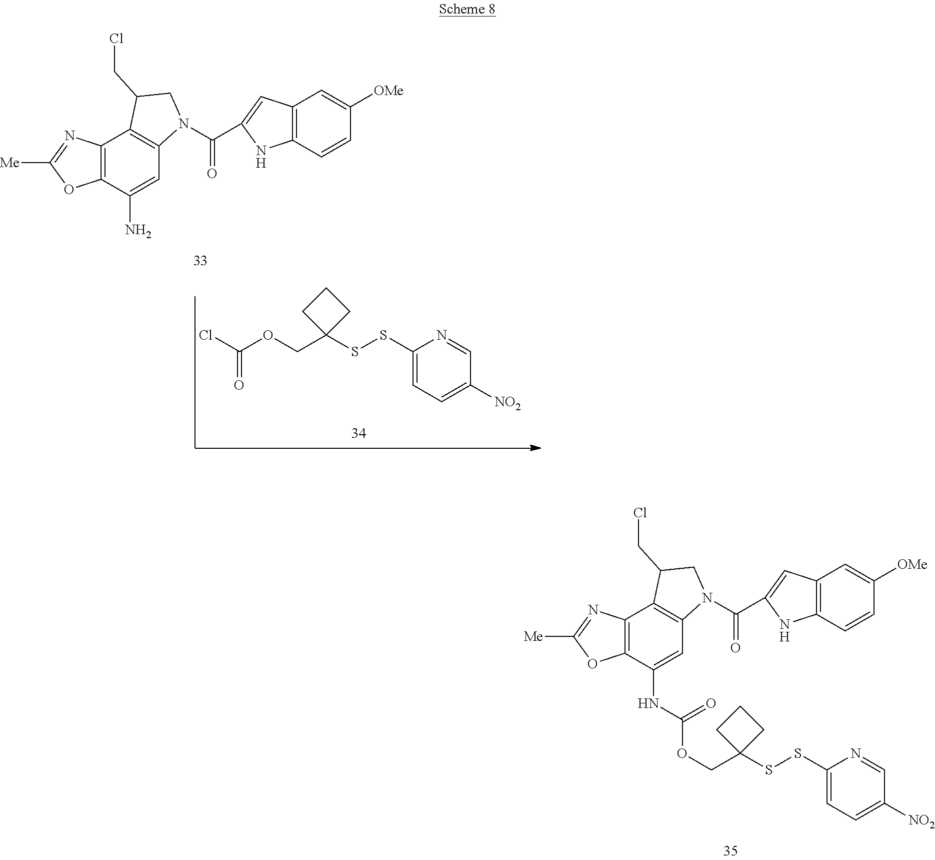 [SMILES: COc1ccc2[nH]c(C(=O)N3CC(CCl)c4c3cc(N)c3oc(C)nc43)cc2c1 ; O=C(Cl)OCC1(SSc2ccc([N+](=O)[O-])cn2)CCC1 ; COc1ccc2[nH]c(C(=O)N3CC(CCl)c4c3cc(NC(=O)OCC3(SSc5ccc([N+](=O)[O-])cn5)CCC3)c3oc(C)nc43)cc2c1]

Compound 33 is reacted with the known disulfide chloroformate reagent 34 (ACS Med. Chem. Lett. (2016) 7, 988) in the presence of pyridine as a base giving compound of the invention 35.

These examples also serve to illustrate that the linker should contain a specific functional group for selective metabolism or reaction in vivo. Examples include dipeptides that are recognised by lysosomal enzymes (e.g. a valine-citrulline or valine-alanine dipeptide that is a recognised cleavage site for cathepsins) or a disulfide bond that is cleaved on exposure to high levels of intracellular reducing agents such as glutathione. The linker should also contain a functional group that allows connection to the antibody, e.g. a maleimide that reacts selectively with thiols such as those produced by the reduction of antibody intrachain disulfide bonds, or those on engineered cysteine residues in site-selectively modified antibodies.

An alternative site for connecting the payload to the linker is at a functional group within the side chain Y. This approach can be used when X═OH or X═prodrug OH. In the latter case the prodrug is usually introduced to the alkylating subunit before the side chain is connected, as illustrated in Scheme 9 below.

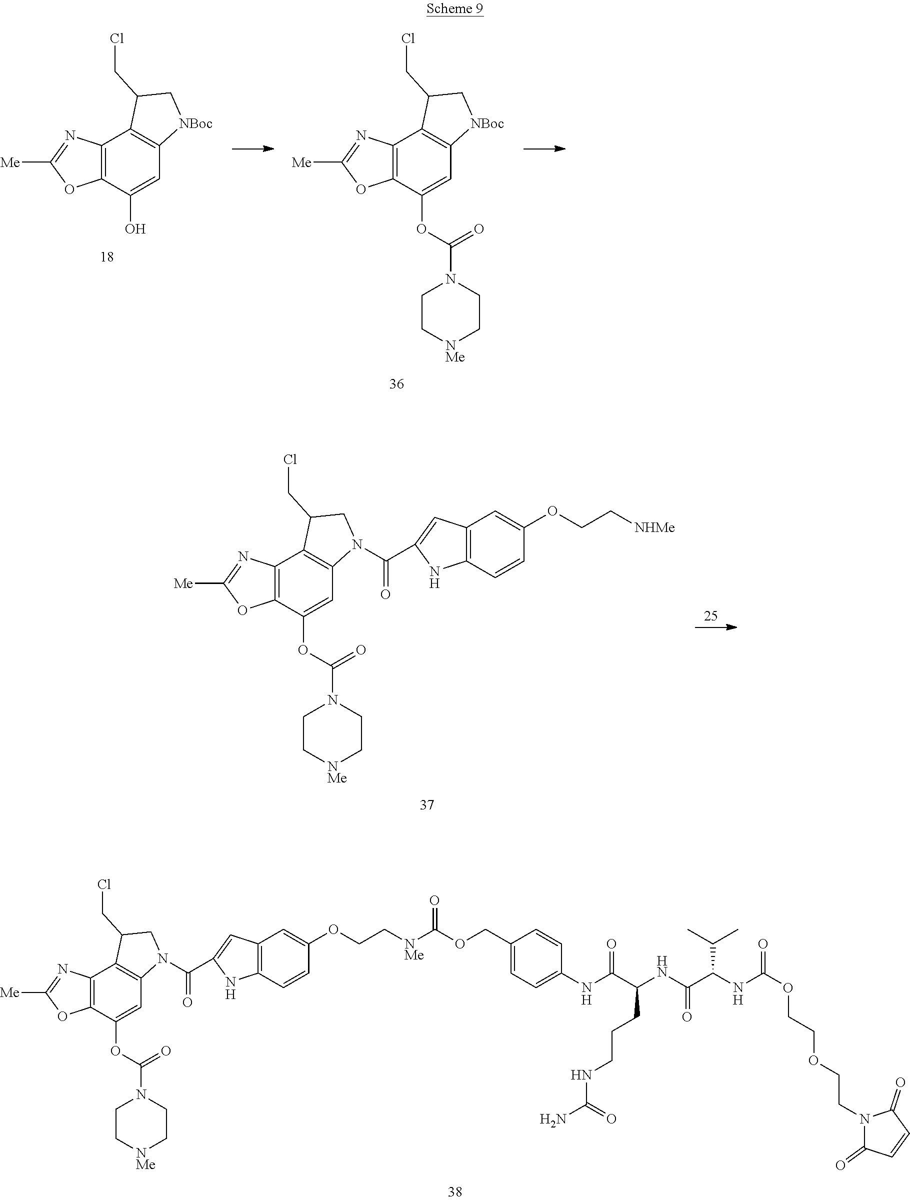 [SMILES: Cc1nc2c3c(cc(O)c2o1)N(C(=O)OC(C)(C)C)CC3CCl ; Cc1nc2c3c(cc(OC(=O)N4CCN(C)CC4)c2o1)N(C(=O)OC(C)(C)C)CC3CCl ; CNCCOc1ccc2[nH]c(C(=O)N3CC(CCl)c4c3cc(OC(=O)N3CCN(C)CC3)c3oc(C)nc43)cc2c1 ; Cc1nc2c3c(cc(OC(=O)N4CCN(C)CC4)c2o1)N(C(=O)c1cc2cc(OCCN(C)C(=O)OCc4ccc(NC(=O)[C@H](CCCNC(N)=O)NC(=O)[C@@H](NC(=O)OCCOCCN5C(=O)C=CC5=O)C(C)C)cc4)ccc2[nH]1)CC3CCl]

Scheme 9 outlines preparation of a compound of the invention where a linker is connected to the side chain via a carbamate. In this scheme, the phenol of the alkylating subunit is protected as a carbamate prodrug. Compound 18 is reacted with 4-methylpiperazinecarbonyl chloride in the presence of DMAP giving compound 36. The Boc protecting group is removed using HCl and the intermediate is reacted with 5-(2-(methylamino)ethoxy)-1H-indole-2-carboxylic acid using EDCI as a coupling reagent to give compound 37. Reaction with the activated linker 25 described above gives drug-linker 38.

When the prodrug is a phosphate OP(O)(OH)₂ the synthesis preferably proceeds using phosphate ester intermediates OP(O)(OR⁴)₂ up until the final step of phosphate ester deprotection. In connecting to the side chain Y there is a lot of flexibility as to how the linker is attached to the side chain. The connection may be traceless, as shown in the scheme above, for which suitable side chain functional groups include alcohols (for carbamate connection), primary and secondary amines (for carbamate connection), tertiary amines (for quaternary salt connection) and thiols (for disulfide connection). These functional groups can be attached to the side chain at any of the identified substituent positions. The connection may also be one that is not traceless, i.e. after cleavage from the ADC the payload still contains a fragment of the linker. This approach is suitable when the linker fragment is of a structure and in a position that does not interfere with alkylation of DNA by the payload, i.e. does not detrimentally impact the cytotoxicity of the released species. Within this limitation non-traceless linkers can be connected to any type of the side chain reactive moieties RM already defined.

### 5.4 Uses of the Compounds of the Invention

The compounds of the invention comprise highly cytotoxic payloads, or payload components, for use in the preparation of ADCs and other biologically active compounds.

Compounds of the invention in which the 2-methylbenzoxazole subunit is attached to a DNA minor groove binding unit, can be converted to ADCs by attaching an antibody or other ligand binding group via a linker. The linker may be attached directly to the DNA alkylating subunit via the hydroxyl or amino group X, or indirectly via the DNA minor groove binding unit.

The binding ligand (e.g., antibody) and appropriate linker can be selected for the particular clinical application the ADC is intended for. The nature of the linker may have an influence over the pharmacokinetic properties of the conjugate, and so should be selected for compatibility with the binding ligand to be used, and the pharmacological requirements of the conjugate as a whole. The linker may include stretcher units, spacer units and moieties to increase solubility.

Examples of such linker groups, stretcher units, and spacer units include but are not limited to those described in U.S. Pat. No. 7,964,566B2 and US2017/0232108A1, which are incorporated by reference herein in their entirety.

In one aspect the invention provides a use of a compound of formula I, Ia, II, IIa, III or IIIa in the preparation of an ADC.

In another aspect, the invention provides a method of making an ADC or ADC component comprising reacting a compound of formula I, Ia, II, IIa, III or IIIa with a linker or linker-antibody moiety.

The new 2-methylbenzoxazole DNA alkylating units are much less lipophilic than the widely used CBI alkylating units which are present in many duocarmycin analogues, as demonstrated in Examples 6 and 24.

This will make the corresponding ADCs easier to manufacture, as the payload component will be more soluble in the aqueous solvents used to conjugate the antibody-linker component. A less lipophilic payload will also cause less aggregation of the ADC which will further simplify manufacture. Reduced aggregation will lessen the risk of an immune response in vivo, and a less lipophilic ADC will have longer clearance from the blood stream and so a greater overall exposure. In summary, a 2-methylbenzoxazole-based payload will generate ADCs that are easier to make, safer and more efficacious.

DNA alkylating agents based on 2-methylbenzoxazole analogues of the duocarmycins are highly cytotoxic compounds. This is demonstrated in Examples 7 and 25 which describe cytotoxicity tests comparing the compounds of the invention with seco-CBI-TMI. The latter is widely recognised as having sufficient cytotoxic potency for application as an ADC payload. The closest comparison between seco-CBI-TMI and compounds of the invention is made with compound 23. Since compound 23 shares the same minor groove binding side chain (i.e. TMI) a direct head-to-head comparison of these two compounds can be made, which demonstrates equivalent cytotoxic potency. The high cytotoxicity of the compounds of the invention is very surprising. The 2-methylbenzoxazole analogues are closely related in structure to known COI duocarmycin analogues (Bioorg. Med. Chem. Lett. (2010) 20, 1854). As illustrated with seco-COI-TMI the only difference between these structures is the orientation of the oxazole ring fusion, and the nature of the 2-substituent (Me versus CO₂Me). Nevertheless, COI duocarmycin analogues are several hundred-fold less cytotoxic than CBI analogues, making COI analogues unsuitable for use as ADC payloads.

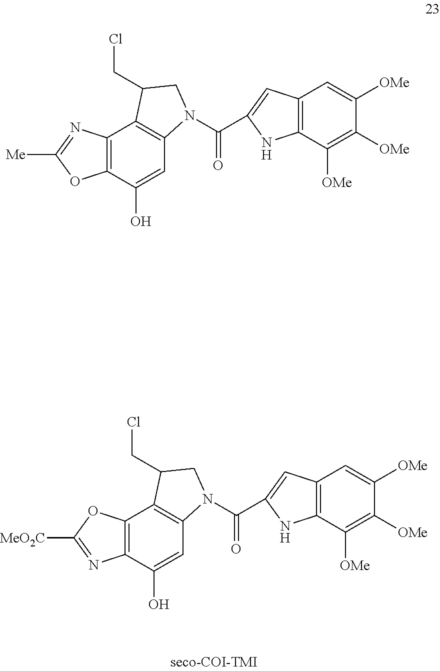 [SMILES: COc1cc2cc(C(=O)N3CC(CCl)c4c3cc(O)c3oc(C)nc43)[nH]c2c(OC)c1OC ; COC(=O)c1nc2c(O)cc3c(c2o1)C(CCl)CN3C(=O)c1cc2cc(OC)c(OC)c(OC)c2[nH]1]

Another advantage of the 2-methoxybenzoxazole duocarmycin analogues of the invention is that they undergo rapid hydrolysis in aqueous buffers under physiological conditions to form inactive products, as demonstrated in Examples 8 and 26.

This means that they are unlikely to persist in circulation very long, and so, if systemic release from an ADC occurs, they are less likely to cause associated off-mechanism toxicity. This stability property is also very surprising because it has been found that cytotoxicity and aqueous stability of duocarmycin analogues are correlated. In other words, all of the most cytotoxic analogues (including CBI) previously reported are also the most stable, and therefore all of the previously reported analogues have the potential to cause undesirable side-effects if released into the general circulation.

While the compounds of the invention are primarily useful as payloads to be incorporated into ADCs, in some embodiments, they may be used as therapeutics in their own right. Therefore, the present invention further relates to a pharmaceutical composition comprising a compound of formula I, Ia, II, IIa, III or IIIa, and a pharmaceutically acceptable carrier.

The term “pharmaceutically acceptable carrier” refers to a carrier (e.g. adjuvant or vehicle) that may be administered to a subject together with the compound of formula I, Ia, II, IIa, III or IIIa, which is generally safe, non-toxic, and neither biologically nor otherwise undesirable, including carriers suitable for veterinary as well as human pharmaceutical use.

Pharmaceutically acceptable carriers that may be used in the compositions include, but are not limited to, ion exchangers, alumina, aluminium stearate, lecithin, self-emulsifying drug delivery systems (SEDDS) such as d-a-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-3-cyclodextrins, or other solubilized derivatives may also be advantageously used to enhance delivery. Oil solutions or suspensions may also contain a long-chain alcohol diluent or dispersant, or carboxymethyl cellulose or similar dispersing agents, which are commonly used in the formulation of pharmaceutically acceptable dosage forms such as emulsions and or suspensions.

The pharmaceutical composition of the invention may be administered as a single dose or in a multiple dose schedule, either as the sole therapeutic agent or simultaneously, sequentially, or separately, in combination with one or more additional therapeutic agents. The one or more additional therapeutic agents will depend on the disease or condition to be treated or other desired therapeutic benefits. The one or more additional therapeutic agents can be used in therapeutic amounts indicated or approved for the particular agent, as would be known to those skilled in the art.

The pharmaceutical compositions are formulated to allow for administration to a subject by any chosen route, including but not limited to oral or parenteral (including topical, subcutaneous, intramuscular and intravenous) administration. In some embodiments, the compositions are formulated for administration orally, intravenously, subcutaneously, intramuscularly, transdermally, intraperitoneally, or other pharmacologically acceptable routes. For example, the compositions may be formulated with an appropriate pharmaceutically acceptable carrier (including excipients, diluents, auxiliaries, and combinations thereof) selected with regard to the intended route of administration and standard pharmaceutical practice. For example, the compositions may be administered orally as a powder, liquid, tablet or capsule, or topically as an ointment, cream or lotion. Suitable formulations may contain additional agents as required, including emulsifying, antioxidant, flavouring or colouring agents, and may be adapted for immediate-, delayed-, modified-, sustained-, pulsed- or controlled-release.

The compositions may be administered via the parenteral route. Examples of parenteral dosage forms include aqueous solutions, isotonic saline or 5% glucose of the active agent, or other well-known pharmaceutically acceptable excipients. Cyclodextrins, for example, or other solubilising agents well-known to those familiar with the art, can be utilized as pharmaceutical excipients for delivery of the therapeutic agent.

Examples of dosage forms suitable for oral administration include, but are not limited to tablets, capsules, lozenges, or like forms, or any liquid forms such as syrups, aqueous solutions, emulsions and the like, capable of providing a therapeutically effective amount of the composition. Capsules can contain any standard pharmaceutically acceptable materials such as gelatin or cellulose. Tablets can be formulated in accordance with conventional procedures by compressing mixtures of the active ingredients with a solid carrier and a lubricant. Examples of solid carriers include starch and sugar bentonite. Active ingredients can also be administered in a form of a hard-shell tablet or a capsule containing a binder, e.g., lactose or mannitol, a conventional filler, and a tabletting agent.

Examples of dosage forms suitable for transdermal administration include, but are not limited, to transdermal patches, transdermal bandages, and the like.

Examples of dosage forms suitable for topical administration of the compositions include any lotion, stick, spray, ointment, paste, cream, gel, etc., whether applied directly to the skin or via an intermediary such as a pad, patch or the like.

Examples of dosage forms suitable for suppository administration of the compositions include any solid dosage form inserted into a bodily orifice particularly those inserted rectally, vaginally and urethrally.

Examples of dosage of forms suitable for injection of the compositions include delivery via bolus such as single or multiple administrations by intravenous injection, subcutaneous, subdermal, and intramuscular administration or oral administration.

Examples of dosage forms suitable for depot administration of the compositions include pellets or solid forms wherein the active(s) are entrapped in a matrix of biodegradable polymers, microemulsions, liposomes or are microencapsulated.

Examples of infusion devices for the compositions include infusion pumps for providing a desired number of doses or steady state administration and include implantable drug pumps. Examples of implantable infusion devices for compositions include any solid form in which the active(s) are encapsulated within or dispersed throughout a biodegradable polymer or synthetic, polymer such as silicone, silicone rubber, silastic or similar polymer.

Examples of dosage forms suitable for transmucosal delivery of the compositions include depositories solutions for enemas, pessaries, tampons, creams, gels, pastes, foams, nebulised solutions, powders and similar formulations containing in addition to the active ingredients such carriers as are known in the art to be appropriate. Such dosage forms include forms suitable for inhalation or insufflation of the compositions, including compositions comprising solutions and/or suspensions in pharmaceutically acceptable, aqueous, or organic solvents, or mixture thereof and/or powders. Transmucosal administration of the compositions may utilize any mucosal membrane but commonly utilizes the nasal, buccal, vaginal and rectal tissues. Formulations suitable for nasal administration of the compositions may be administered in a liquid form, for example, nasal spray, nasal drops, or by aerosol administration by nebulizer, including aqueous or oily solutions of the polymer particles. Formulations may be prepared as aqueous solutions for example in saline, solutions employing benzyl alcohol or other suitable preservatives, absorption promoters to enhance bio-availability, fluorocarbons, and/or other solubilising or dispersing agents known in the art.

Examples of dosage forms suitable for buccal or sublingual administration of the compositions include lozenges, tablets and the like. Examples of dosage forms suitable for opthalmic administration of the compositions include inserts and/or compositions comprising solutions and/or suspensions in pharmaceutically acceptable, aqueous, or organic solvents.

Examples of formulations of compositions may be found in, for example, Sweetman, S. C. (Ed.). Martindale. The Complete Drug Reference, 33rd Edition, Pharmaceutical Press, Chicago, 2002, 2483 pp.; Aulton, M. E. (Ed.) Pharmaceutics. The Science of Dosage Form Design. Churchill Livingstone, Edinburgh, 2000, 734 pp.; and, Ansel, H. C, Allen, L. V. and Popovich, N. G. Pharmaceutical Dosage Forms and Drug Delivery Systems, 7th Ed., Lippincott 1999, 676 pp. Excipients employed in the manufacture of drug delivery systems are described in various publications known to those skilled in the art including, for example, Kibbe, E. H. Handbook of Pharmaceutical Excipients, 3rd Ed., American Pharmaceutical Association, Washington, 2000, 665 pp. The USP also provides examples of modified-release oral dosage forms, including those formulated as tablets or capsules. See, for example, The United States Pharmacopeia 23/National Formulary 18, The United States Pharmacopeial Convention, Inc., Rockville Md., 1995 (hereinafter “the USP”), which also describes specific tests to determine the drug release capabilities of extended-release and delayed-release tablets and capsules. The USP test for drug release for extended-release and delayed-release articles is based on drug dissolution from the dosage unit against elapsed test time. Descriptions of various test apparatus and procedures may be found in the USP. Further guidance concerning the analysis of extended release dosage forms has been provided by the F.D.A. (See Guidance for Industry. Extended release oral dosage forms: development, evaluation, and application of in vitro/in vivo correlations. Rockville, Md.: Center for Drug Evaluation and Research, Food and Drug Administration, 1997).

The dosage forms described herein can be in the form of physically discrete units suitable for use as unitary dosages for the subjects to be treated, each unit containing a predetermined quantity of active material calculated to produce the desired therapeutic effect.

Dosage levels of the active ingredients in the pharmaceutical compositions may be varied so as to provide an amount of the active ingredient which is effective to achieve the desired therapeutic effect for a particular patient, composition, and mode of administration, without being toxic to the patient (an effective amount).

The selected dosage level will depend upon a variety of pharmacokinetic factors including the activity of the particular compositions employed, the route of administration, the time of administration, the rate of excretion of the particular compound of the invention being employed, other drugs, compounds and/or materials used in combination with the particular compositions employed, the age, sex, weight, condition, general health and prior medical history of the patient being treated, and like factors well known in the medical arts. Generally, the daily amount or regimen should be in the range of about 0.01 mg to about 2000 mg of the compound of the invention per kilogram (kg) of body mass.

### 6. EXAMPLES

General Methods and Materials

All reagents were purchased as reagent grade and used without further purification. Solvents for reactions were distilled prior to use according to standard procedures. Petroleum ether refers to the fraction with bp 40-60° C. Solvents were dried according to standard procedures, i.e. over anhydrous Na₂SO₄ or MgSO₄. Column chromatography was carried out on silica gel (Merck 230-400 mesh).

NMR spectra were recorded on a Bruker Avance 400 MHz instrument for 1H and 100 MHz for ¹³C spectra chemical shifts are reported in parts per million (ppm) and calibrated to tetramethylsilane (0 ppm) as an internal standard in 1H spectra, and residual solvent in ¹³C spectra. Multiplicities are reported as follows: br=broad, s=singlet, d=doublet, t=triplet, q=quartet, m=multiplet, dd=doublet of doublets, dt=doublet of triplets, ddd=doublet of doublet of doublets. High-resolution mass spectra (HRMS) were obtained using a Bruker microTOF-Q II or an Agilent 6530B Accurate Mass Q-TOF mass spectrometer. LRMS was performed with a Surveyor MSQ mass spectrometer.

Unless alternative general methods and materials are given, the above general materials and methods are applicable to all of the examples below.

### Example 1. tert-Butyl 6-methyl-4-oxo-8,8a-dihydro-1H-cyclopropa[c]oxazolo[4,5-e]indole-2(4H)-carboxylate (50)

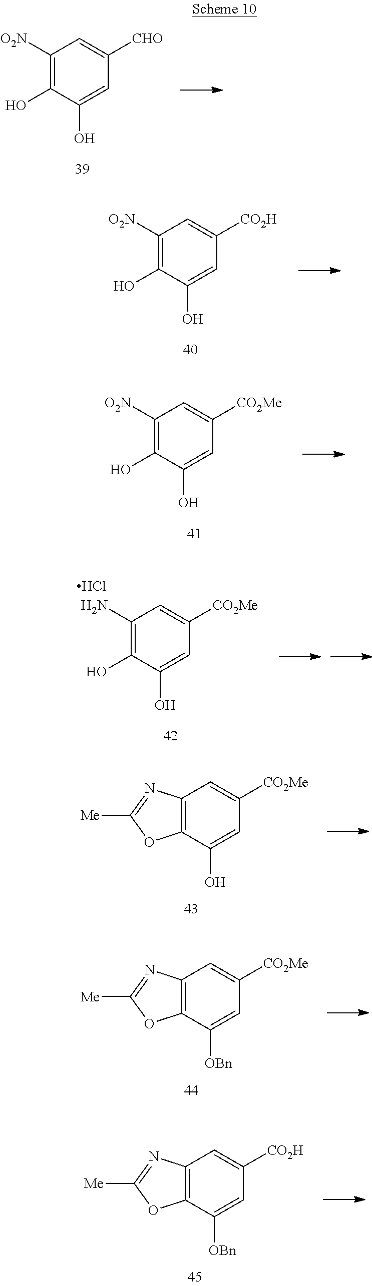

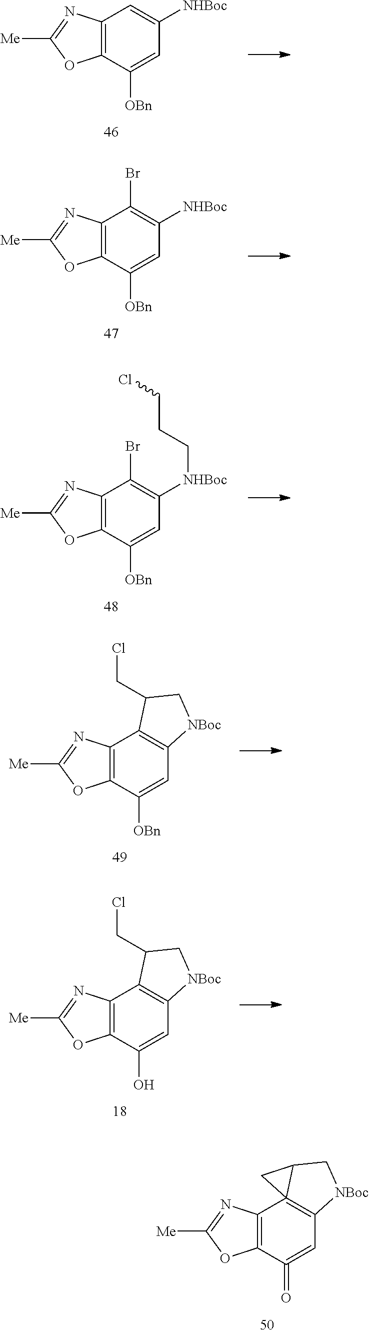

A solution of NaH₂PO₄.2H₂O (4.29 g, 27.5 mmol) in H₂O (10 mL) was added to a solution of 3,4-dihydroxy-5-nitrobenzaldehyde 39 (5.03 g, 27.5 mmol) in DMSO (25 mL). To the stirred mixture was added a solution of NaClO₂ (80%, 4.35 g, 38.5 mmol) in H₂O (25 mL) dropwise over 50 min keeping the internal temperature below 45° C. The dark red-brown mixture was stirred at room temperature for 16 h and then poured into aqueous NaHCO₃ (5%, 80 mL). The mixture was washed with CH₂Cl₂ (×2) and the aqueous phase was acidified with c.HCl to give pH ˜1. The mixture was extracted with EtOAc (×3) and the combined extracts were washed with brine and then dried and evaporated to give 3,4-dihydroxy-5-nitrobenzoic acid (40) as a yellow-brown solid (5.12 g, 94%); ¹H NMR (d₆-DMSO) δ 13.02 (br s, 1H), 10.80 (br s, 2H), 7.87 (d, J=2.0 Hz, 1H), 7.57 (d, J=2.0 Hz, 1H).

A mixture of 40 (3.99 g, 20.0 mmol) in MeOH (60 mL) and c.H₂SO₄ (1.5 mL) was stirred at reflux for 19 h and then cooled and evaporated. The residue was partitioned between EtOAc and brine (×2). The EtOAc extracts were dried and evaporated and the resulting solid was recrystallized from H₂O to give methyl 3,4-dihydroxy-5-nitrobenzoate (41) as an orange-brown crystalline solid (3.30 g, 77%); mp 137-139° C.; ¹H NMR (d₆-DMSO) δ 10.84 (br s, 2H), 7.89 (d, J=2.1 Hz, 1H), 7.57 (d, J=2.1 Hz, 1H), 3.83 (s, 3H).

HCl in dioxane (4M, 2.94 mL, 11.8 mmol) and Pd/C (10%, 0.25 g) were added to a solution of 41 (2.51 g, 11.8 mmol) in EtOH (35 mL). The mixture was degassed with nitrogen, flushed with hydrogen, and then stirred under a hydrogen balloon until the reduction was complete (˜8 h). The mixture was filtered through Celite, washing with EtOH, and the filtrate was evaporated to give methyl 3-amino-4,5-dihydroxybenzoate hydrochloride (42) as a pale yellow solid (3.0 g) which was used directly in the next step. Anal. (C₈H₁₀NO₄) calculated for [M+H]⁺ 184.1; found 184.1.

Trimethyl orthoacetate (13.6 mL, 107 mmol) was added to a portion of 42 (2.71 g) prepared as described above. The suspension was stirred at reflux for 1 h, then cooled and diluted with petroleum ether. The precipitate was filtered off and dried to give methyl 7-hydroxy-2-methylbenzo[d]oxazole-5-carboxylate (43) as a light tan solid (2.02 g, 91% from 41); ¹H NMR (d₆-DMSO) δ 10.76 (s, 1H), 7.66 (d, J=1.5 Hz, 1H), 7.43 (d, J=1.5 Hz, 1H), 3.85 (s, 3H), 2.62 (s, 3H); ¹³C NMR (d₆-DMSO) δ 166.1, 165.0, 143.0, 142.2, 141.9, 126.5, 112.1, 111.1, 52.2, 14.1. Anal. (C₁₀H₉NO₄) calculated for [M+H]⁺ 208.06043; found 208.06061.

Benzyl bromide (98%, 1.24 mL, 10.2 mmol) and K₂CO₃ (1.48 g, 10.7 mmol) were added to a solution of 43 (2.02 g, 9.75 mmol) in DMF (20 mL) and the mixture was stirred at room temperature for 18 h. The mixture was diluted with H₂O and the solid was filtered off and dried to give methyl 7-(benzyloxy)-2-methylbenzo[d]oxazole-5-carboxylate (44) (2.81 g, 97%) as a light tan solid; ¹H NMR (d₆-DMSO) δ 7.83 (d, J=1.3 Hz, 1H), 7.63 (d, J=1.3 Hz, 1H), 7.54-7.49 (m, 2H), 7.46-7.34 (m, 3H), 5.36 (s, 2H), 3.87 (s, 3H), 2.63 (s, 3H); ¹³C NMR (d₆-DMSO) δ 165.9, 165.4, 143.0, 142.8, 142.6, 136.1, 128.5, 128.2, 128.0, 126.7, 113.2, 109.5, 70.5, 52.4, 14.1. Anal. (C₁₇H₁₅NO₄) calculated for [M+H]⁺ 298.10738; found 298.10834.

A solution of KOH (1.62 g, 29.1 mmol) in H₂O (15 mL) was added to a suspension of 44 (2.79 g, 9.38 mmol) in MeOH (60 mL) and the mixture was stirred at 70° C. for 40 min. The MeOH was evaporated and the aqueous remainder was acidified with aqueous HCl (2N, 12 mL) at 0° C. The precipitated solid was filtered off and dried to give 7-(benzyloxy)-2-methylbenzo[d]oxazole-5-carboxylic acid (45) as an off-white solid (2.62 g, 98%); ¹H NMR (d₆-DMSO) δ 13.06 (br s, 1H), 7.81 (d, J=1.3 Hz, 1H), 7.62 (d, J=1.3 Hz, 1H), 7.53-7.48 (m, 2H), 7.46-7.34 (m, 3H), 5.36 (s, 2H), 2.63 (s, 3H). Anal. (C₁₆H₁₃NO₄) calculated for [M+H]⁺ 284.09173; found 284.09164.

Et₃N (1.54 mL, 11.1 mmol) and DPPA (97%, 2.25 mL, 10.1 mmol) were added to a suspension of 45 (2.61 g, 9.21 mmol) in dry tert-BuOH (80 mL) and the mixture was stirred at reflux for 3 h. The solvent was evaporated and the residue was purified by column chromatography (petroleum ether:EtOAc 9:1 then 4:1) to give tert-butyl (7-(benzyloxy)-2-methylbenzo[d]oxazol-5-yl)carbamate (46) as a cream solid (2.44 g, 75%); ¹H NMR (d₆-DMSO) δ 9.37 (s, 1H), 7.52-7.47 (m, 2H), 7.44-7.33 (m, 4H), 7.21 (s, 1H), 5.21 (s, 2H), 2.56 (s, 3H), 1.48 (s, 9H); ¹³C NMR (d₆-DMSO) δ 164.1, 152.8, 142.8, 142.4, 136.8, 136.3, 135.1, 128.5, 128.2, 128.1, 100.72, 100.67, 79.1, 70.3, 28.1, 14.1. Anal. (C₂₀H₂₂N₂O₄) calculated for [M+H]⁺ 355.1652; found 355.1668. A sample was recrystallized from EtOAc/petroleum ether to give white needles, mp 150-152 OC.

NBS (1.16 g, 6.52 mmol) was added in portions over 10 min to a solution of 46 (2.31 g, 6.52 mmol) in CH₃CN (100 mL) at 0° C. The mixture was stirred at room temperature for 3 h and then the solvent was evaporated. The residue was dissolved in EtOAc and the solution was washed with H₂O (×2), then with brine, and then dried and evaporated. The resulting cream solid was recrystallized from EtOAc/petroleum ether to give tert-butyl (7-(benzyloxy)-4-bromo-2-methylbenzo[d]oxazol-5-yl)carbamate (47) as a white solid (2.15 g, 76%); mp 154-156° C.; ¹H NMR (CDCl₃) δ 8.01 (s, 1H), 7.53-7.47 (m, 2H), 7.43-7.32 (m, 3H), 7.03 (s, 1H), 5.27 (s, 2H), 2.65 (s, 3H), 1.55 (s, 9H); ¹³C NMR (CDCl₃) δ 165.0, 152.9, 143.0, 142.0, 136.4, 136.1, 133.9, 128.8, 128.6, 128.3, 102.2, 92.4, 81.4, 71.6, 28.5, 14.8. Anal. (C₂₀H₂₁⁷⁹BrN₂O₄) calculated for [M+H]⁺ 433.0757; found 433.0762; (C₂₀H₂₁⁸¹BrN₂O₄) calculated for [M+H]⁺ 435.0740; found 435.0745. The mother liquor was evaporated and the residue was purified by column chromatography (petroleum ether:EtOAc 4:1) to give more 47 (0.60 g, 21%).

1,3-Dichloropropene (mixed isomers, 90%, 1.92 mL, 18.6 mmol) and K₂CO₃ (4.3 g, 31 mmol) were added to a solution of 47 (2.69 g, 6.21 mmol) in DMF (12 mL) and the mixture was stirred at 80° C. for 7 h. The DMF was evaporated and the residue was partitioned between EtOAc and H₂O. The aqueous layer was extracted again with EtOAc (×2) and the combined EtOAc layers were washed with brine (×3) and then dried and evaporated to give crude tert-butyl (7-(benzyloxy)-4-bromo-2-methylbenzo[d]oxazol-5-yl)(3-chloroallyl)carbamate (48) as a light brown gum (3.21 g, 100%); ¹H NMR (CDCl₃) (mixture of E and Z isomers and Boc rotamers) δ 7.50-7.32 (m, 5H), 6.85-6.66 (m, 1H), 6.05-5.88 (m, 2H), 5.34-5.15 (m, 2H), 4.52-4.44, 4.42-4.26, 4.06-4.03, 3.89-3.80 (4×m, 2H), 2.69 (s, 3H), 1.56 (s, 9H). Anal. (C₂₃H₂₄⁷⁹BrClN₂O₄) calculated for [M+H]⁺ 507.06807; found 507.06909.

Bu₃SnH (97%, 1.09 mL, 3.92 mmol) and AIBN (64 mg, 0.39 mmol) were added to a solution of 48 (995 mg, 1.96 mmol) in dry toluene (15 mL) under nitrogen and the mixture was stirred at reflux. Further portions of Bu₃SnH (97%, 0.54 mL, 2.0 mmol) and AIBN (32 mg, 0.2 mmol) were added after 1.5 and 3 h. After 4 h the toluene was evaporated, and the residue was partitioned between CH₃CN and petroleum ether. The petroleum ether layer was extracted again with CH₃CN (×2) and the combined extracts were washed with petroleum ether (×2) and then evaporated. The residue was purified by column chromatography (petroleum ether:EtOAc 9:1 then 6:1) to give the crude product as a white foam. This was stirred with petroleum ether (8 mL) to give tert-butyl 4-(benzyloxy)-8-(chloromethyl)-2-methyl-7,8-dihydro-6H-oxazolo[4,5-e]indole-6-carboxylate (49) as a white solid (668 mg, 80%); mp 142-143° C.; ¹H NMR (CDCl₃) δ 7.78 (br s, 1H), 7.52-7.45 (m, 2H), 7.43-7.31 (m, 3H), 5.26 (s, 2H), 4.21 (dd, J=11.5, 9.7 Hz, 1H), 4.15-3.96 (m, 3H), 3.65 (t, J=9.9 Hz, 1H), 2.62 (s, 3H), 1.57 (s, 9H); ¹³C NMR (CDCl₃) δ 165.0, 152.6, 143.7, 141.4, 139.3, 136.9, 136.4, 128.8, 128.4, 128.1, 111.6, 97.6, 81.2, 71.4, 53.2, 46.8, 40.9, 28.7, 14.8. Anal. (C₂₃H₂₅ClN₂O₄) calculated for [M+H]⁺ 429.15756; found 429.15735.

An aqueous solution of NH₄HCO₂ (25%, 3.9 mL, 15.4 mmol) and Pd/C (10%, wetted with 53% H₂O, 0.13 g) were added to a solution of 48 (660 mg, 1.54 mmol) in THF (10 mL) under nitrogen at 0° C., and the mixture was stirred vigorously at this temperature. Further portions of aqueous NH₄HCO₂ (25%, 3.9 mL, 15.4 mmol) and Pd/C (10%, wetted with 53% H₂O, 0.13 g) were added after 3 h. After 7 h the mixture was filtered through Celite, washing with EtOAc. The EtOAc layer from the filtrate was washed with brine and then dried and evaporated. The residue was recrystallized from EtOAc/petroleum ether to give tert-butyl 8-(chloromethyl)-4-hydroxy-2-methyl-7,8-dihydro-6H-oxazolo[4,5-e]indole-6-carboxylate (18) as a white solid (444 mg, 85%); mp 234-238° C.; ¹H NMR (CDCl₃) δ 7.66 (br s, 1H), 7.63 (br s, 1H), 4.18 (dd, J=11.5, 9.6 Hz, 1H), 4.11-3.95 (m, 3H), 3.63 (apparent t, J=9.9 Hz, 1H), 2.62 (s, 3H), 1.56 (s, 9H); ¹³C NMR (CDCl₃) δ 165.2, 153.0, 141.1, 140.9, 139.2, 136.1, 111.1, 99.9, 81.7, 53.3, 46.9, 40.8, 28.7, 14.8. Anal. (C₁₆H₁₉ClN₂O₄) calculated for [M+H]⁺ 339.11061; found 339.11149.

K₂CO₃ (41 mg, 0.3 mmol) was added to a solution of 18 (67 mg, 0.2 mmol) in DMF (1 mL) and the mixture was stirred at room temperature for 3.5 h, then diluted with EtOAc and H₂O. The EtOAc layer was washed with brine (×3) and then dried and evaporated. The residue was purified by column chromatography (petroleum ether:EtOAc 2:3 then 1:4) to give 50 as a colourless oil (22 mg, 37%); ¹H NMR (CDCl₃) δ 6.72 (s, 1H), 4.05 (d, J=11.4 Hz, 1H), 3.99 (dd, J=11.4, 5.0 Hz, 1H), 2.94-2.88 (m, 1H), 2.57 (s, 3H), 2.01 (dd, J=7.8, 4.0 Hz, 1H), 1.55 (s, 9H), 1.46 (dd, J=4.9, 4.4 Hz, 1H); ¹³C NMR (CDCl₃) δ 175.1, 165.5, 158.6, 151.7, 148.1, 145.8, 109.8, 83.6, 53.7, 33.1, 28.3, 24.9, 23.4, 14.6. Anal. (C₁₆H₁₈N₂O₄) calculated for [M+H]⁺ 303.13393; found 303.13458.

### Example 2. (8-(Chloromethyl)-4-hydroxy-2-methyl-7,8-dihydro-6H-oxazolo[4,5-e]indol-6-yl)(5,6,7-trimethoxy-1H-indol-2-yl)methanone (23)

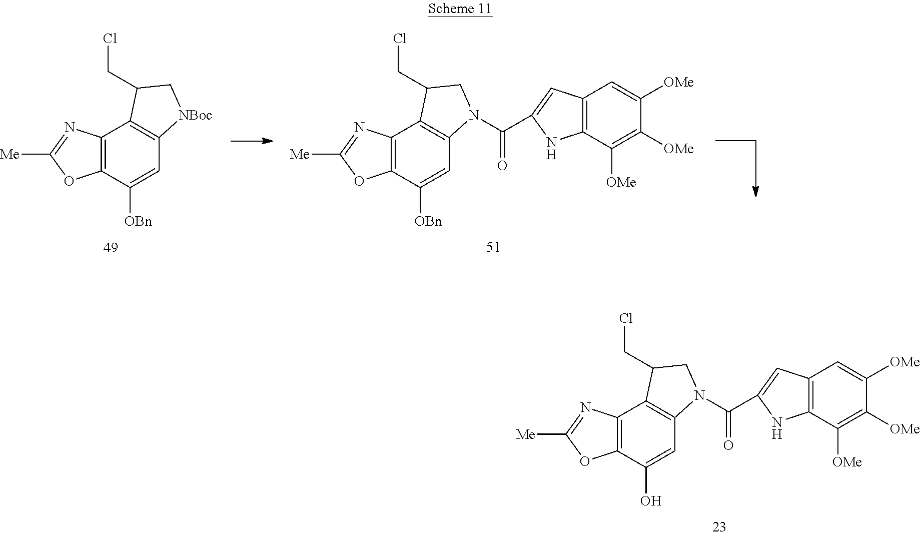

A mixture of 49 (51 mg, 0.12 mmol) and HCl in dioxane (4M, 1 mL) was stirred at room temperature for 3 h and then evaporated. The residue was suspended in DMA (1.5 mL) and 5,6,7-trimethoxy-1H-indole-2-carboxylic acid (31.4 mg, 0.13 mmol) and EDCI.HCl (46 mg, 0.24 mmol) were added. The mixture was stirred at room temperature for 1 h and then dilute aqueous NaHCO₃ was added. The precipitated solid was filtered off, washed with H₂O, and dried. The crude product was recrystallized from EtOAc to give (4-(benzyloxy)-8-(chloromethyl)-2-methyl-7,8-dihydro-6H-oxazolo[4,5-e]indol-6-yl)(5,6,7-trimethoxy-1H-indol-2-yl)methanone (51) as an off-white solid (34 mg, 51%); mp 225-227° C.; ¹H NMR (d₆-DMSO) δ 11.44 (d, J=1.4 Hz, 1H), 8.04 (br s, 1H), 7.53-7.47 (m, 2H), 7.45-7.33 (m, 3H), 7.03 (d, J=2.0 Hz, 1H), 6.97 (s, 1H), 5.27 (s, 2H), 4.74 (t, J=10.2 Hz, 1H), 4.40 (dd, J=10.9, 5.3 Hz, 1H), 4.25-4.16 (m, 1H), 4.13 (dd, J=10.9, 3.1 Hz, 1H), 4.06-4.00 (m, 1H), 3.93 (s, 3H), 3.83 (s, 3H), 3.78 (s, 3H), 2.64 (s, 3H); ¹³C NMR (d₆-DMSO) δ 165.2, 160.1, 149.2, 142.3, 141.5, 139.9, 139.1, 138.5, 136.7, 136.3, 130.8, 128.6, 128.2, 127.9, 125.3, 123.2, 113.1, 106.1, 70.5, 61.1, 61.0, 56.0, 54.6, 46.8, 40.7, 14.2. Anal. (C₃₀H₂₈ClN₃O₆) calculated for [M+H]⁺ 562.17394; found 562.17311.

An aqueous solution of NH₄HCO₂ (25%, 0.14 mL, 0.55 mmol) and Pd/C (10%, wetted with 53% H₂O, 34 mg) were added to a solution of 51 (31 mg, 0.055 mmol) in THF (20 mL) under nitrogen at 0° C., and the mixture was stirred vigorously at this temperature. More aqueous NH₄HCO₂ (25%, 0.28 mL, 1.1 mmol) and Pd/C (10%, wetted with 53% H₂O, 35 mg) were added after 1 h. After 2 h the mixture was filtered through Celite, washing with THF, and the filtrate was evaporated to dryness at 30° C. The residue was triturated with H₂O and the solid was filtered off and dried. A further trituration with EtOAc gave 23 as a white solid (21 mg, 81%); mp 285° C. (dec); ¹H NMR (d₆-DMSO) δ 11.39 (d, J=1.6 Hz, 1H), 10.45 (s, 1H), 7.84 (s, 1H), 7.01 (d, J=2.1 Hz, 1H), 6.96 (s, 1H), 4.71 (t, J=10.1 Hz, 1H), 4.36 (dd, J=10.9, 5.1 Hz, 1H), 4.17-4.09 (m, 2H), 3.97 (dd, J=11.2, 7.8 Hz, 1H), 3.94 (s, 3H), 3.82 (s, 3H), 3.78 (s, 3H), 2.61 (s, 3H); ¹³C NMR (d₆-DMSO) δ 164.8, 156.0, 149.2, 141.3, 141.2, 139.8, 139.1, 138.6, 136.2, 131.0, 125.2, 123.2, 111.0, 105.9, 101.4, 98.0, 61.1, 61.0, 56.0, 54.7, 46.9, 40.7, 14.2. Anal. (C₂₃H₂₂ClN₃O₆) calculated for [M+H]⁺ 472.12699; found 472.12694.

### Example 3. (8-(Chloromethyl)-4-hydroxy-2-methyl-7,8-dihydro-6H-oxazolo[4,5-e]indol-6-yl)(5-(2-(dimethylamino)ethoxy)-1H-indol-2-yl)methanone (52)

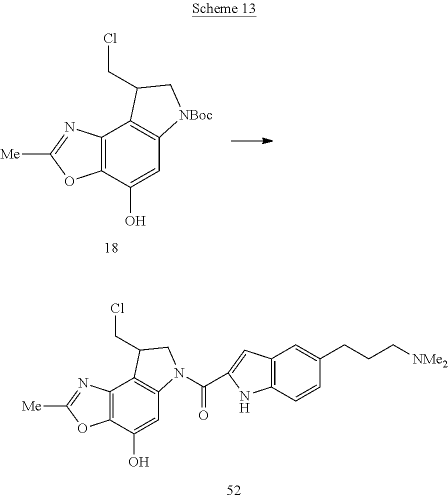 [SMILES: Cc1nc2c3c(cc(O)c2o1)N(C(=O)OC(C)(C)C)CC3CCl ; Cc1nc2c3c(cc(O)c2o1)N(C(=O)c1cc2cc(CCCN(C)C)ccc2[nH]1)CC3CCl]

A mixture of 18 (33 mg, 0.097 mmol) and HCl in dioxane (4M, 1 mL) was stirred at room temperature for 4 h and then evaporated. The residue was suspended in DMA (1.0 mL) and 5-(2-(dimethylamino)ethoxy)-1H-indole-2-carboxylic acid hydrochloride (34 mg, 0.12 mmol), EDCI.HCl (65 mg, 0.34 mmol), and anhydrous toluenesulfonic acid (3.3 mg, 0.019 mmol) were added. The mixture was stirred at room temperature for 1.5 h and then cooled in an ice bath. EtOAc (50 mL) and H₂O (10 mL) were added and the pH was adjusted to 8-9 using saturated aqueous NaHCO₃. The EtOAc layer was separated, washed with water, and then dried and evaporated. The residue was purified by column chromatography (CH₂Cl₂:MeOH 10:1) to give 52 (34 mg, 75%); mp 206-210° C. (dec); ¹H NMR (d₆-DMSO) δ 11.59 (br s, 1H), 10.46 (br s, 1H), 7.91 (s, 1H), 7.38 (d, J=8.9 Hz, 1H), 7.17 (d, J=2.3 Hz, 1H), 7.05 (d, J=1.7 Hz, 1H), 6.92 (dd, J=8.9, 2.4 Hz, 1H), 4.76 (t, J=10.2 Hz, 1H), 4.48-4.40 (m, 1H), 4.22-4.06 (m, 4H), 4.04-3.95 (m, 1H), 2.80 (poorly resolved t, J=5.3 Hz, 2H), 2.61 (s, 3H), 2.34 (s, 6H); ¹³C NMR (d₆-DMSO) δ 164.7, 159.8, 152.8, 141.28, 141.25, 138.6, 136.2, 131.6, 131.0, 127.5, 115.7, 113.1, 111.0, 105.1, 103.3, 101.6, 65.6, 57.4, 54.5, 46.9, 45.1, 40.8, 14.2. Anal (C₂₄H₂₅ClN₄O₄) calculated for [M+H]⁺ 469.1637; found 469.1642.

### Example 4. N-(2-(8-(Chloromethyl)-4-hydroxy-2-methyl-7,8-dihydro-6H-oxazolo[4,5-e]indole-6-carbonyl)-1H-indol-5-yl)-5-(2-(dimethylamino)ethoxy)-1H-indole-2-carboxamide (53)

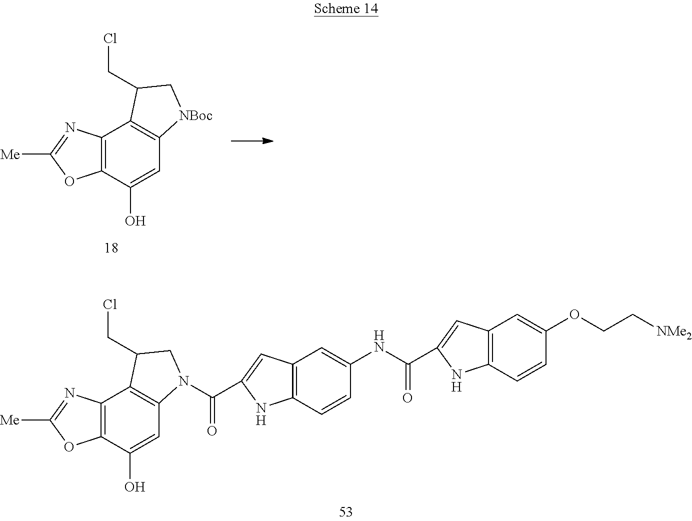 [SMILES: Cc1nc2c3c(cc(O)c2o1)N(C(=O)OC(C)(C)C)CC3CCl ; Cc1nc2c3c(cc(O)c2o1)N(C(=O)c1cc2cc(NC(=O)c4cc5cc(OCCN(C)C)ccc5[nH]4)ccc2[nH]1)CC3CCl]

A mixture of 18 (33 mg, 0.097 mmol) and HCl in dioxane (4M, 1 mL) was stirred at room temperature for 4 h and then evaporated. The residue was suspended in DMA (1.0 mL) and 5-(5-(2-(dimethylamino)ethoxy)-1H-indole-2-carboxamido)-1H-indole-2-carboxylic acid hydrochloride (43 mg, 0.097 mmol), EDCI.HCl (65 mg, 0.34 mmol), and anhydrous toluenesulfonic acid (3.3 mg, 0.019 mmol) were added. The mixture was stirred at room temperature for 1.5 h and then cooled in an ice bath. EtOAc (150 mL) and H₂O (50 mL) were added and the pH was adjusted to 8-9 using saturated aqueous NaHCO₃. The EtOAc layer was separated, washed with water, and then dried and evaporated. The residue was purified by column chromatography (CH₂Cl₂:MeOH 10:1 then 5:1) to give 53 (46 mg, 75%); mp 231° C. (dec); ¹H NMR (d₆-DMSO) δ 11.70 (br s, 1H), 11.59 (br s, 1H), 10.47 (br s, 1H), 10.13 (s, 1H), 8.21 (d, J=1.5 Hz, 1H), 7.93 (s, 1H), 7.57 (dd, J=8.9, 1.9 Hz, 1H), 7.46 (d, J=8.8 Hz, 1H), 7.41-7.27 (m, 2H), 7.22-7.11 (m, 2H), 6.87 (dd, J=8.9, 2.4 Hz, 1H), 4.80 (t, J=10.2, 1H), 4.53-4.43 (m, 1H), 4.24-3.98 (m, 5H), 2.77 (poorly resolved t, J=5.4 Hz, 2H), 2.62 (s, 3H), 2.34 (s, 6H); ¹³C NMR (d₆-DMSO) δ 164.8, 159.8, 159.5, 152.8, 141.3, 141.2, 138.6, 138.6, 136.3, 133.2, 132.3, 132.1, 131.3, 127.4, 127.1, 119.3, 115.1, 113.2, 112.9, 112.2, 111.1, 105.6, 103.2, 103.1, 101.6, 65.6, 57.5, 54.5, 46.9, 45.2, 40.8, 39.5, 14.2. Anal (C₃₃H₃₁ClN₆O₅) calculated for [M+H]⁺ 627.2117; found 627.2119.

### Example 5. N-(2-(8-(Chloromethyl)-4-hydroxy-2-methyl-7,8-dihydro-6H-oxazolo[4,5-e]indole-6-carbonyl)imidazo[1,2-a]pyridin-6-yl)-4-hydroxybenzamide (57)

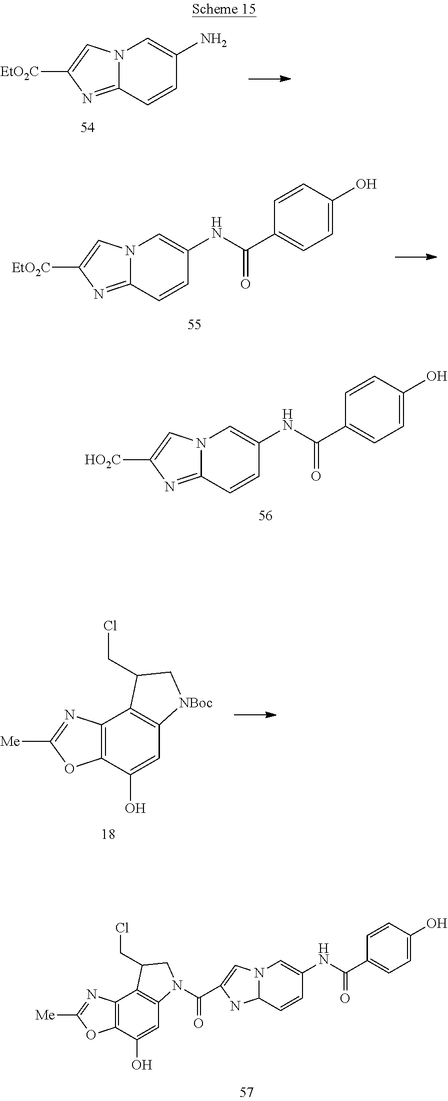 [SMILES: CCOC(=O)c1cn2cc(N)ccc2n1 ; CCOC(=O)c1cn2cc(NC(=O)c3ccc(O)cc3)ccc2n1 ; O=C(Nc1ccc2nc(C(=O)O)cn2c1)c1ccc(O)cc1 ; Cc1nc2c3c(cc(O)c2o1)N(C(=O)OC(C)(C)C)CC3CCl ; Cc1nc2c3c(cc(O)c2o1)N(C(=O)C1=CN2C=C(NC(=O)c4ccc(O)cc4)C=CC2N1)CC3CCl]

A mixture of ethyl 6-aminoimidazo[1,2-a]pyridine-2-carboxylate (54) (50 mg, 0.24 mmol), 4-hydroxybenzoic acid (68 mg, 0.49 mmol), EDCI.HCl (163 mg, 0.85 mmol), and anhydrous toluenesulfonic acid (8.7 mg, 0.05 mmol) in DMA (1 mL) was stirred at room temperature for 2 h. The mixture was diluted with H₂O and extracted with EtOAc. The EtOAc layer was dried and evaporated and the residue was purified by column chromatography (EtOAc only then EtOAc:MeOH 10:1) to give ethyl 6-(4-hydroxybenzamido)imidazo[1,2-a]pyridine-2-carboxylate (55) as a greenish-amber solid (41 mg, 52%); mp 263-266° C.; ¹H NMR (ds-DMSO) δ 10.17, 10.16 (partially overlapping s, 2H), 9.40-9.35 (m, 1H), 8.64 (s, 1H), 7.93-7.84 (m, 2H), 7.62 (d, J=9.7 Hz, 1H), 7.55 (dd, J=9.8, 1.9 Hz, 1H), 6.93-6.84 (m, 2H), 4.30 (q, J=7.1 Hz, 2H), 1.32 (t, J=7.1 Hz, 3H); 13C NMR (ds-DMSO) δ 165.3, 162.7, 160.8, 142.3, 135.6, 129.8, 127.6, 124.5, 123.5, 118.9, 117.5, 117.2, 115.0, 60.1, 14.3. Anal (C₁₇H₁₅N₃O₄) calculated for [M+H]⁺ 326.1135; found 326.1125.

Solid KOH (171 mg, 3.05 mmol) was added to a solution of 55 (198 mg, 0.61 mmol) in THF (6 mL), MeOH (6 mL), and H₂O (3 mL) and the mixture was stirred at room temperature for 1 h and then evaporated. The residue was diluted with water (10 mL) and acidified with aqueous HCl (5M) to pH<1. The solid was filtered off, washed with H₂O. and dried to give 6-(4-hydroxybenzamido)imidazo[1,2-a]pyridine-2-carboxylic acid (56) as a yellow solid (143 mg, 79%), mp 268-271° C.; ¹H NMR (d₆-DMSO) δ 10.17 (s, 2H), 9.40 (s, 1H), 8.59 (s, 1H), 7.95-7.84 (m, 2H), 7.63 (d, J=9.7 Hz, 1H), 7.56 (dd, J=9.7, 1.9 Hz, 1H), 6.96-6.83 (m, 2H), 1H not observed; ¹³C NMR (d₆-DMSO) δ 165.3, 163.9, 160.8, 142.0, 136.1, 129.8, 127.6, 124.6, 123.5, 118.8, 117.3, 117.2, 115.0. Anal (C₁₅H₁₁N₃O₄) calculated for [M+H]⁺ 298.0822; found 298.0818.

A mixture of 18 (38 mg, 0.11 mmol) and HCl in dioxane (4M, 1 mL) was stirred at room temperature for 3 h 15 min and then evaporated. The residue was suspended in DMA (1.0 mL) and 56 (28 mg, 0.093 mmol), EDCI.HCl (63 mg, 0.33 mmol), and anhydrous toluenesulfonic acid (3.3 mg, 0.019 mmol) were added. The mixture was stirred at room temperature for 1.5 h. EtOAc (150 mL) and H₂O (50 mL) were added and the EtOAc layer was separated, washed with H₂O (×2), and then dried and evaporated. The residue was triturated with MeOH to give 57 (30 mg, 63%); mp 232-235° C.; ¹H NMR (d₆-DMSO) δ 10.41 (s, 1H), 10.17 (s, 2H), 9.42 (br s, 1H), 8.62 (s, 1H), 7.97 (br s, 1H), 7.88 (d, J=8.7 Hz, 2H), 7.69 (d, J=9.7 Hz, 1H), 7.56 (dd, J=9.7, 2.0 Hz, 1H), 6.90 (d, J=8.7 Hz, 2H), 4.93 (t, J=10.7 Hz, 1H), 4.73-4.62 (m, 1H), 4.18-4.07 (m, 2H), 4.03-3.92 (m, 1H), 2.61 (s, 3H); ¹³C NMR (d₆-DMSO) δ 165.3, 164.7, 161.4, 160.8, 141.4, 141.3, 141.2, 140.9, 138.6, 136.2, 129.8, 127.5, 124.6, 123.0, 118.6, 117.4, 117.1, 115.0, 111.1, 101.5, 54.8, 46.9, 40.7, 14.2. Anal (C₂₆H₂₀ClN₅O₅) calculated for [M+H]⁺ 518.1226; found 518.1223.

### Example 6: Lipophilicity of Compounds of the Invention

The lipophilicity was calculated for the representative compounds of the invention using the ChemDraw Professional v.17.0.0 software package (Perkin Elmer Informatics Inc). The results are shown in Table 1.

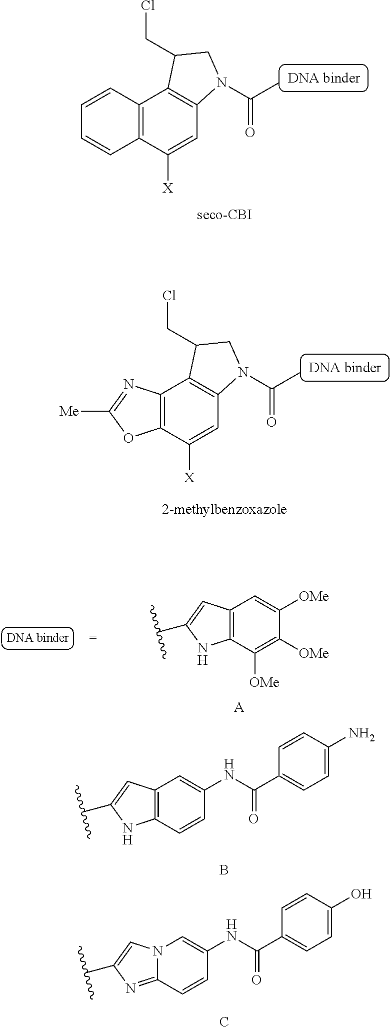

The compounds of the invention bear a variety of DNA minor groove binding side chains: side chain A is the 5,6,7-trimethoxyindole structure found in the duocarmycin natural products; side chain B has been used in the payload of the ADC BMS-936561 (Biopharm. Drug Disp. (2016) 37, 93); and side chain C has been used in the payload seco-DUBA in the ADC SYD985 (Mol. Pharm. (2015) 12, 1813).

In every case the compounds containing the 2-methylbenzoxazole alkylating subunit have a substantially lower calculated log P than those incorporating the seco-CBI alkylating subunit (on the order of 1.5 units), and this applies whether the alkylating subunit is in the form of a phenol (X═OH) or an amine (X═NH₂).

These substantial reductions in payload lipophilicity are highly likely to lead to favourable properties for drug-linkers and ADCs which incorporate the 2-methylbenzoaxazole alkylating subunit.

TABLE 1
Lipophilicity comparison
LogP

| DNA | | | 2- |
|---|---|---|---|
| alkylating | DNA | Seco- | Methyl |
| agents | binder | CBI | benzoxazole |
| Phenols | A | 3.36 | 1.88 |
| (X = OH) | B | 3.74 | 2.26 |
| | C | 3.84 | 2.36 |
| Amines | A | 2.94 | 1.46 |
| (X = NH₂) | B | 3.32 | 1.85 |
| | C | 3.42 | 1.95 |

### Example 7: Cytotoxicity

The cytotoxicity of the payloads of the invention was determined by measuring the inhibition of proliferation of two human tumour cell lines, the cervical carcinoma SiHa, and the ovarian carcinoma SKOV3. Log-phase monolayers were exposed continuously to the payloads for 5 days in 96-well plates, followed by sulforhodamine B staining. The IC₅₀ was determined by interpolation as the drug concentration required to inhibit cell density to 50% of that of the untreated controls on the same plate. Every plate contained the reference compound seco-CBI-TMI as an internal control.

The data presented in Table 2 show that DNA alkylating agents containing the 2-methylbenzoxazole alkylating subunit are highly cytotoxic compounds, with IC₅₀s in the nM or sub-nM range, i.e. within the range considered suitable for ADC application.

Importantly compounds 23 and seco-CBI-TMI have the same TMI minor groove binding side chain, allowing a head-to-head comparison of the effect of the alkylating subunit on cytotoxicity. In this comparison the 2-methylbenzoxazole alkylating subunit produces the same cytotoxicity as seco-CBI, which is notable since the latter is one of the most potent of all known variants of the duocarmycin-type alkylating subunits (J. Med. Chem. (2009) 52, 5771).

The marked cytotoxicity of 23 is surprising because the closely related compound seco-COI-TMI (which differs only in the orientation of the oxazole ring fusion and the nature of the 2-substituent) was several hundred fold less cytotoxic than anticipated when compared to related reference compounds (Bioorg. Med. Chem. Lett. (2010) 20, 1854).

TABLE 2
Cytotoxicity comparison

| | IC₅₀ (nM) | |
|---|---|---|
| Compound | SiHa | SKOV3 |
| Seco- | 0.23 | 0.58 |
| CBI-TMI | | |
| 23 | 0.28 | 0.54 |
| 52 | 0.48 | 1.1 |
| 53 | 7.2 | 9.0 |
| 57 | 5.7 | 12 |

### Example 8: Stability

The aqueous stability of 23 and reference compound seco-CBI-TMI was investigated by LC-MS analysis. Samples containing the payloads at a concentration of 4 μM in Tris buffer (pH 7.4) containing 10% DMF at 37° C. were monitored at regular intervals over 300-500 min.

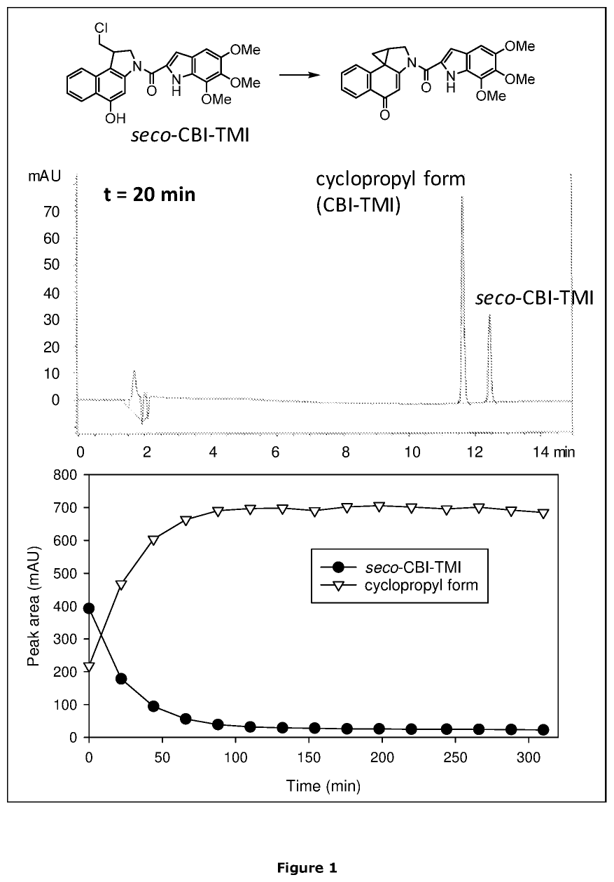

As shown in FIG. 1, seco-CBI-TMI underwent clean conversion to the cyclopropyl form (CBI-TMI), which was stable under these conditions. These observations are consistent with reported behavior (ChemBioChem (2014) 15, 1998; J. Am. Chem. Soc. (1994) 116, 7996).

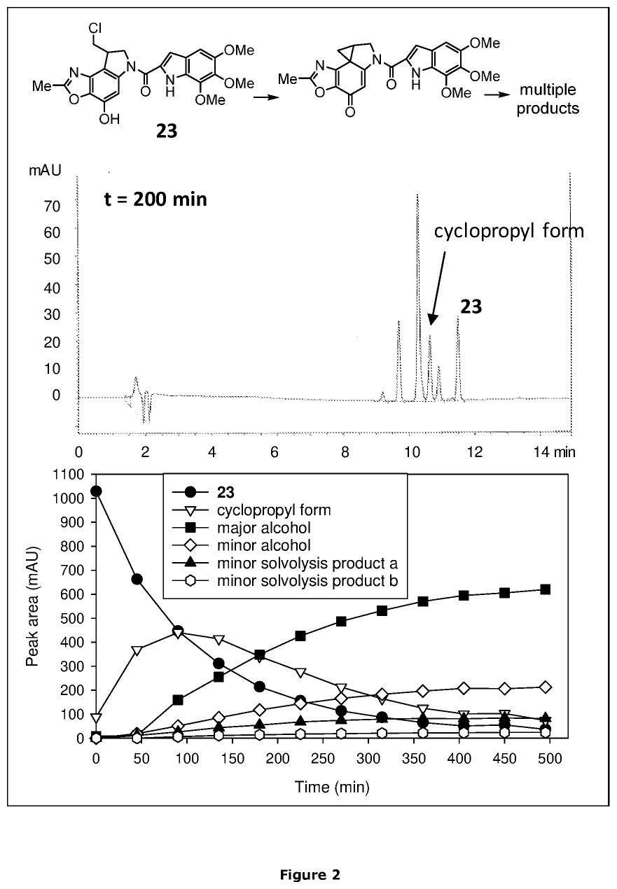

In contrast, as shown in FIG. 2, although 23 also underwent conversion to the corresponding cyclopropyl form (identified by characteristic UV-Vis absorption spectrum and mass spectrum) this compound was not stable under these conditions and was rapidly converted to a variety of products. The major products had UV-Vis absorption spectra and mass spectra consistent with hydrolysis, i.e. ring opening of the cyclopropane by addition of water. Such products are incapable of alkylating DNA and can be considered non-toxic. The instability of the cyclopropyl intermediate derived from the 2-methylbenzoxazole alkylating subunit is very surprising, since there is a well-established correlation between solvolytic stability and cytotoxic potency and all other known alkylating subunits that are as cytotoxic as CBI are reported to be stable to hydrolysis at neutral pH (J. Med. Chem. (2009) 52, 5771). Instability of payloads containing the 2-methylbenzoxazole alkylating subunit may be an advantage in ADC applications by serving as a detoxifying mechanism for any systemically released payload.

### Example 9. (E)-1-(8-(Chloromethyl)-4-hydroxy-2-methyl-7,8-dihydro-6H-oxazolo[4,5-e]indol-6-yl)-3-(4-methoxyphenyl)prop-2-en-1-one (58)

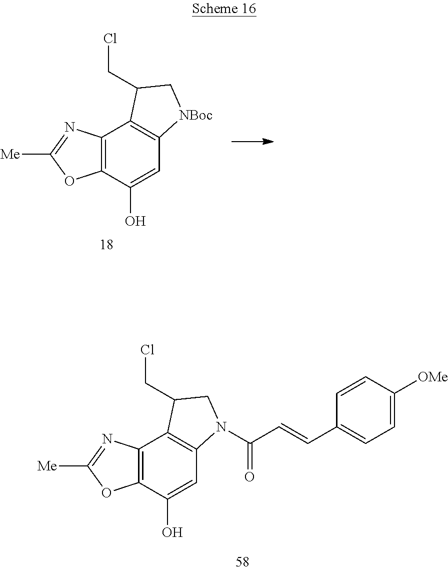 [SMILES: Cc1nc2c3c(cc(O)c2o1)N(C(=O)OC(C)(C)C)CC3CCl ; COc1ccc(/C=C/C(=O)N2CC(CCl)c3c2cc(O)c2oc(C)nc32)cc1]

A mixture of 18 (31.3 mg, 0.092 mmol) and HCl in dioxane (4M, 1 mL) was stirred at room temperature for 4 h and then evaporated. The residue was suspended in DMA (1.5 mL) and 4-methoxycinnamic acid (18 mg, 0.10 mmol) and EDCI.HCl (53 mg, 0.28 mmol) were added. The mixture was stirred at room temperature for 19 h. Water was added slowly and the precipitated solid was filtered off and dried to give 58 as a light brown solid (10 mg, 27%); ¹H NMR (d₆-DMSO) δ 10.39 (s, 1H), 7.96 (s, 1H), 7.80-7.70 (m, 2H), 7.60 (d, J=15.3 Hz, 1H), 7.07-6.97 (m, 3H), 4.63-4.54 (m, 1H), 4.32-4.25 (m, 1H), 4.17-4.08 (m, 2H), 3.98 (dd, J=10.8, 7.5 Hz, 1H), 3.31 (s, 3H), 2.60 (s, 3H). Anal (C₂₁H₁₉ClN₂O₄) calculated for [M+H]⁺ 399.1106; found 399.1108.

### Example 10. (E)-1-(8-(Chloromethyl)-4-hydroxy-2-methyl-7,8-dihydro-6H-oxazolo[4,5-e]indol-6-yl)-3-(3-hydroxy-4-methoxyphenyl) prop-2-en-1-one (59)

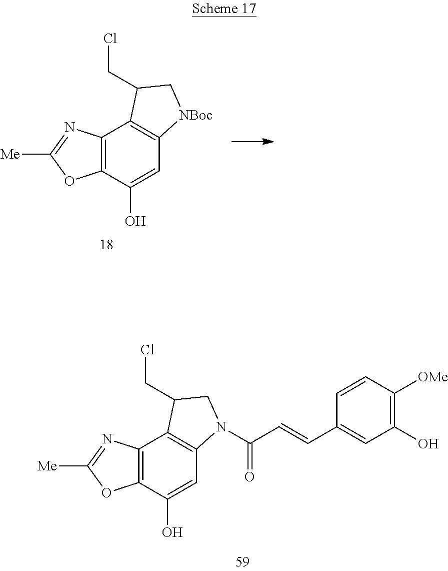 [SMILES: Cc1nc2c3c(cc(O)c2o1)N(C(=O)OC(C)(C)C)CC3CCl ; COc1ccc(/C=C/C(=O)N2CC(CCl)c3c2cc(O)c2oc(C)nc32)cc1O]

A mixture of 18 (30.1 mg, 0.089 mmol) and HCl in dioxane (4M, 1 mL) was stirred at room temperature for 4 h and then evaporated. The residue was suspended in DMA (1.0 mL) and 3-hydroxy-4-methoxycinnamic acid (19 mg, 0.10 mmol) and EDCI.HCl (51 mg, 0.27 mmol) were added. The mixture was stirred at room temperature for 3 h, then diluted with water and extracted with EtOAc (×2). The combined extracts were washed with water (×3) and brine, and then dried and evaporated. The residue was triturated with a small volume of EtOAc (ca. 1 mL) to give 59 as a very pale green solid (7.7 mg, 21%); ¹H NMR (d₆-DMSO) δ 10.41 (s, 1H), 9.14 (s, 1H), 7.96 (s, 1H), 7.51 (d, J=15.3 Hz, 1H), 7.24 (d, J=2.0 Hz, 1H), 7.16 (dd, J=8.4, 2.0 Hz, 1H), 6.96 (d, J=8.4 Hz, 1H), 6.91 (d, J=15.3 Hz, 1H), 4.63-4.54 (m, 1H), 4.31-4.24 (m, 1H), 4.16-4.06 (m, 2H), 3.95 (dd, J=11.0, 7.8 Hz, 1H), 3.83 (s, 3H), 2.60 (s, 3H); ¹³C NMR (d₆-DMSO) δ 164.6, 163.5, 149.6, 146.6, 142.4, 141.3, 141.2, 138.6, 136.0, 127.8, 121.2, 117.2, 114.2, 111.9, 110.7, 101.1, 55.6, 52.8, 47.0, 40.3, 14.2. Anal (C₂₁H₁₉ClN₂O₅) calculated for [M+H]⁺ 415.1055; found 415.1060.

### Example 11. (E)-N-(2-(8-(Chloromethyl)-4-hydroxy-2-methyl-7,8-dihydro-6H-oxazolo[4,5-e]indole-6-carbonyl)-1H-indol-5-yl)-3-(4-methoxyphenyl)acrylamide (63)

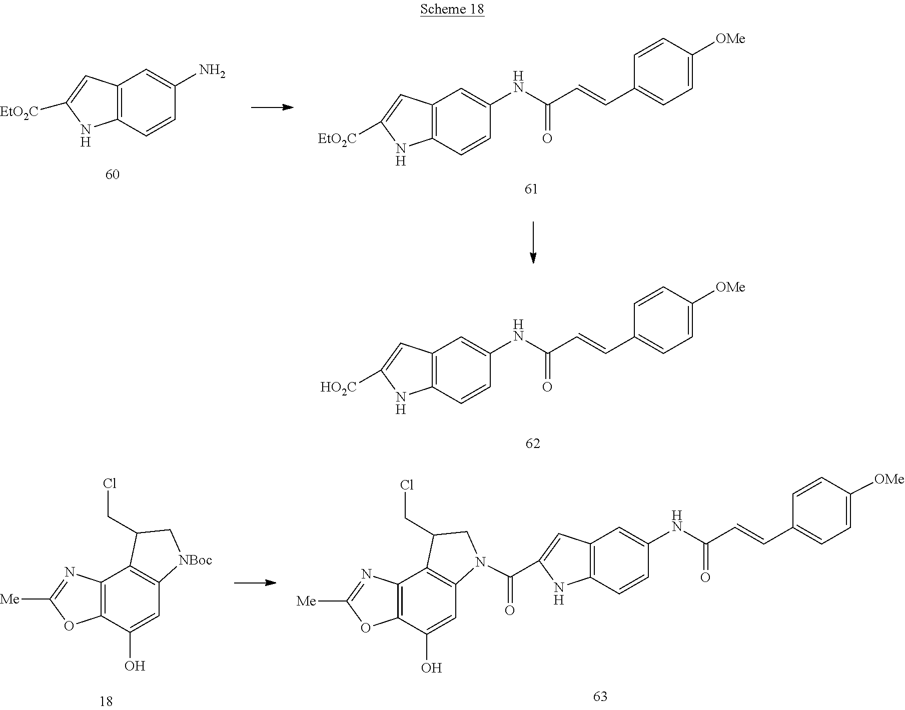 [SMILES: CCOC(=O)c1cc2cc(N)ccc2[nH]1 ; CCOC(=O)c1cc2cc(NC(=O)/C=C/c3ccc(OC)cc3)ccc2[nH]1 ; COc1ccc(/C=C/C(=O)Nc2ccc3[nH]c(C(=O)O)cc3c2)cc1 ; Cc1nc2c3c(cc(O)c2o1)N(C(=O)OC(C)(C)C)CC3CCl ; COc1ccc(/C=C/C(=O)Nc2ccc3[nH]c(C(=O)N4CC(CCl)c5c4cc(O)c4oc(C)nc54)cc3c2)cc1]

A mixture of ethyl 5-aminoindole-2-carboxylate (60) (354 mg, 1.73 mmol), 4-methoxycinnamic acid (309 mg, 1.73 mmol) and EDCI.HCl (0.67 g, 3.46 mmol) in DMA (4 mL) was stirred at room temperature for 18 h. Water was added and the precipitated solid was filtered off, washed with water, and dried. The crude product was stirred with hot EtOH (40 mL) and the suspension was cooled. The solid was filtered off and dried to give ethyl (E)-5-(3-(4-methoxyphenyl)acrylamido)-1H-indole-2-carboxylate (61) as a cream solid (506 mg, 80%); mp 244-247° C.; ¹H NMR (d₆-DMSO) δ 11.82 (s, 1H), 10.03 (s, 1H), 8.15 (s, 1H), 7.58 (d, J=8.6 Hz, 2H), 7.53 (d, J=15.6 Hz, 1H), 7.48-7.37 (m, 2H), 7.12 (s, 1H), 7.02 (d, J=8.6 Hz, 2H), 6.70 (d, J=15.6 Hz, 1H), 4.34 (q, J=7.1 Hz, 2H), 3.80 (s, 3H), 1.35 (t, J=7.1 Hz, 3H). Anal (C₂₁H₂₀N₂O₄¼H₂O) calculated for C, 68.37; H, 5.60; N, 7.59%; found C, 68.56; H, 5.53; N, 7.66%.

A solution of KOH (0.40 g, 7.1 mmol) in water (6 mL) was added to a suspension of 61 (480 mg, 1.32 mmol) in EtOH (15 mL) and the mixture was heated at reflux for 5 min. The solution was cooled to room temperature and acidified with aqueous HCl (2N, 5 mL). The resulting suspension was diluted with water and the solid was filtered off, washed with water, and dried to give (E)-5-(3-(4-methoxyphenyl)acrylamido)-1H-indole-2-carboxylic acid (62) as a light tan solid (414 mg, 93%); mp 276-279° C.; ¹H NMR (d₆-DMSO) δ 12.90 (br s, 1H), 11.68 (s, 1H), 10.01 (s, 1H), 8.14 (s, 1H), 7.57 (d, J=8.8 Hz, 2H), 7.53 (d, J=15.6 Hz, 1H), 7.45-7.35 (m, 2H), 7.06 (d, J=1.8 Hz, 1H), 7.01 (d, J=8.8 Hz, 2H), 7.21 (d, J=15.6 Hz, 1H), 3.80 (s, 3H). Anal (C₁₉H₁₆N₂O₄) calculated for C, 67.85; H, 4.79; N, 8.33%; found C, 68.09; H, 4.77; N, 8.39%.

A mixture of 18 (30.0 mg, 0.089 mmol) and HCl in dioxane (4M, 1 mL) was stirred at room temperature for 4 h and then evaporated. The residue was suspended in DMA (1.0 mL) and 62 (33 mg, 0.10 mmol) and EDCI.HCl (51 mg, 0.27 mmol) were added. The mixture was stirred at room temperature for 3 h. Water was added slowly and the precipitated solid was filtered off and dried. The crude product was triturated with EtOAc to give 63 as a light tan solid (24 mg, 49%); ¹H NMR (d₆-DMSO) δ 11.67 (s, 1H), 10.46 (s, 1H), 10.04 (s, 1H), 8.20 (s, 1H), 7.91 (s, 1H), 7.58 (d, J=8.8 Hz, 2H), 7.54 (d, J=15.6 Hz, 1H), 7.43-7.36 (m, 2H), 7.15 (d, J=2.1 Hz, 1H), 7.02 (d, J=8.8 Hz, 2H), 6.72 (d, J=15.6 Hz, 1H), 4.80 (t, J=10.2 Hz, 1H), 4.47 (dd, J=10.9, 5.1 Hz, 1H), 4.23-4.10 (m, 2H), 4.06-3.99 (m, 1H), 3.80 (s, 3H), 2.62 (s, 3H); ¹³C NMR (d₆-DMSO) δ 164.8, 163.5, 160.5, 159.8, 141.3, 141.2, 139.1, 138.6, 136.3, 133.0, 132.3, 131.3, 129.2, 127.5, 127.2, 120.2, 118.1, 114.5, 112.3, 111.5, 111.1, 105.6, 101.6, 55.3, 54.5, 46.9, 40.8, 14.2. Anal (C₃₀H₂₅ClN₄O₅) calculated for [M+H]⁺ 557.1586; found 557.1593.

### Example 12. (8-(Chloromethyl)-4-hydroxy-2-methyl-7,8-dihydro-6H-oxazolo[4,5-e]indol-6-yl)(5-methoxybenzofuran-2-yl)methanone (64)

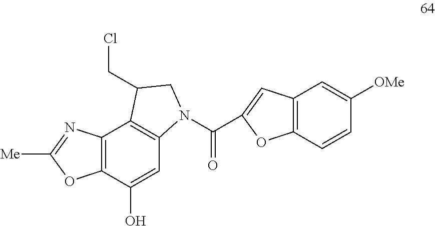 [SMILES: COc1ccc2oc(C(=O)N3CC(CCl)c4c3cc(O)c3oc(C)nc43)cc2c1]

Compound 64 was prepared from 18 by the same general method as set out in Examples 3 and 4 and isolated as a grey solid; ¹H NMR (d₆-DMSO) δ 10.50 (s, 1H), 7.85 (br s, 1H), 7.65 (d, J=9.2 Hz, 1H), 7.63 (s, 1H), 7.30 (d, J=2.6 Hz, 1H), 7.10 (dd, J=9.0, 2.6 Hz, 1H), 4.79 (t, J=10.4 Hz, 1H), 4.46 (dd, J=11.2, 5.1 Hz, 1H), 4.20-4.09 (m, 2H), 4.02 (dd, J=10.7, 7.2 Hz, 1H), 3.33 (s, 3H), 2.62 (s, 3H). Anal (C₂₁H₁₇ClN₂O₅) calculated for [M+H]⁺ 413.0899; found 413.0899.

### Example 13. (8-(Chloromethyl)-4-hydroxy-2-methyl-7,8-dihydro-6H-oxazolo[4,5-e]indol-6-yl)(5-methoxybenzo[b]thiophen-2-yl)methanone (65)

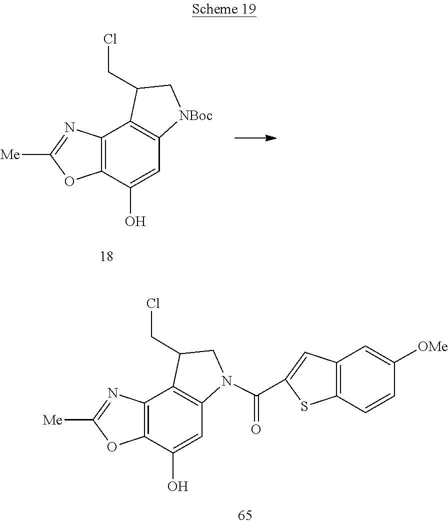 [SMILES: Cc1nc2c3c(cc(O)c2o1)N(C(=O)OC(C)(C)C)CC3CCl ; COc1ccc2sc(C(=O)N3CC(CCl)c4c3cc(O)c3oc(C)nc43)cc2c1]

A mixture of 18 (40.0 mg, 0.12 mmol) and HCl in dioxane (4M, 2 mL) was stirred at room temperature for 1.5 h and then evaporated. The residue was suspended in DMA (1.5 mL) and 5-methoxybenzo[b]thiophene-2-carboxylic acid (25 mg, 0.12 mmol), EDCI.HCl (68 mg, 0.36 mmol) and toluenesulfonic acid (4 mg, 0.024 mmol) were added. The mixture was stirred at room temperature for 3 h. Water was added slowly and the precipitated solid was filtered off and dried. The crude product was triturated with EtOAc to give 65 as a cream solid (35 mg, 69%); ¹H NMR (d₆-DMSO) δ 10.49 (s, 1H), 8.02 (s, 1H), 7.94 (d, J=8.9 Hz, 1H), 7.79 (br s, 1H), 7.53 (d, J=2.5 Hz, 1H), 7.14 (dd, J=8.9, 2.5 Hz, 1H), 4.74 (t, J=10.0 Hz, 1H), 4.40 (dd, J=10.8, 5.2 Hz, 1H), 4.20-4.08 (m, 2H), 4.05-3.98 (m, 1H), 3.85 (s, 3H), 2.62 (s, 3H); ¹³C NMR (d₆-DMSO) δ 164.9, 160.7, 157.4, 141.3, 140.8, 140.21, 140.17, 138.6, 136.5, 132.2, 126.5, 123.3, 117.1, 111.4, 106.9, 101.5, 55.3, 55.1, 46.7, 40.8, 14.2. Anal (C₂₁H₁₇ClN₂O₄S) calculated for [M+H]⁺ 429.0670; found 429.0677.

### Example 14. (8-(Chloromethyl)-4-hydroxy-2-methyl-7,8-dihydro-6H-oxazolo[4,5-e]indol-6-yl)(5-(2-methoxyethoxy)-1H-indol-2-yl)methanone (66)

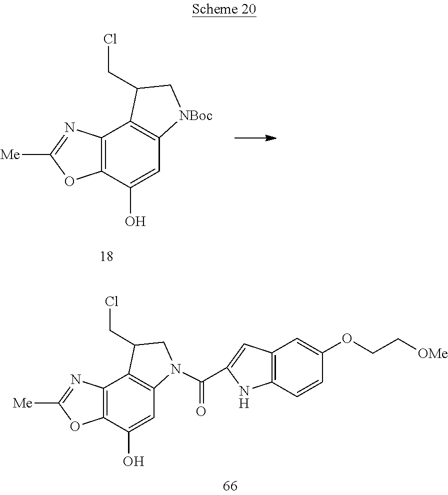 [SMILES: Cc1nc2c3c(cc(O)c2o1)N(C(=O)OC(C)(C)C)CC3CCl ; COCCOc1ccc2[nH]c(C(=O)N3CC(CCl)c4c3cc(O)c3oc(C)nc43)cc2c1]

A mixture of 18 (36 mg, 0.106 mmol) and HCl in dioxane (4M, 1 mL) was stirred at room temperature for 4 h and then evaporated. The residue was suspended in DMA (1.0 mL) and 5-(2-methoxyethoxy)indole-2-carboxylic acid (27.5 mg, 0.12 mmol), EDCI.HCl (61 mg, 0.32 mmol) and toluenesulfonic acid (3.7 mg, 0.021 mmol) were added. The mixture was stirred at room temperature for 2 h. Water was added slowly and the precipitated solid was filtered off, washed with water, and dried. The crude product was triturated with EtOAc to give 66 as a grey solid (28 mg, 58%); ¹H NMR (d₆-DMSO) δ 11.58 (d, J=1.5 Hz, 1H), 10.45 (s, 1H), 7.92 (s, 1H), 7.39 (d, J=8.9 Hz, 1H), 7.16 (d, J=2.3 Hz, 1H), 7.04 (d, J=1.7 Hz, 1H), 6.90 (dd, J=8.9, 2.4 Hz, 1H), 4.78 (t, J=10.1 Hz, 1H), 4.45 (dd, J=10.8, 5.1 Hz, 1H), 4.21-4.06 (m, 2H), 4.04-3.97 (m, 1H), 3.71-3.66 (m, 2H), 2.62 (s, 3H) (one 3H s obscured by water peak at δ 3.3); ¹³C NMR (d₆-DMSO) δ 164.8, 159.9, 152.9, 141.3, 141.2, 138.6, 136.3, 131.6, 131.0, 127.5, 115.7, 113.2, 111.0, 105.2, 103.1, 101.6, 70.6, 67.2, 58.2, 54.6, 46.9, 40.9, 14.2. Anal (C₂₃H₂₂ClN₃O₅) calculated for [M+H]⁺ 456.1321; found 456.1323.

### Example 15. N-(2-(8-(Chloromethyl)-4-hydroxy-2-methyl-7,8-dihydro-6H-oxazolo[4,5-e]indole-6-carbonyl)-1H-pyrrolo[2,3-b]pyridin-5-yl)acetamide (70)

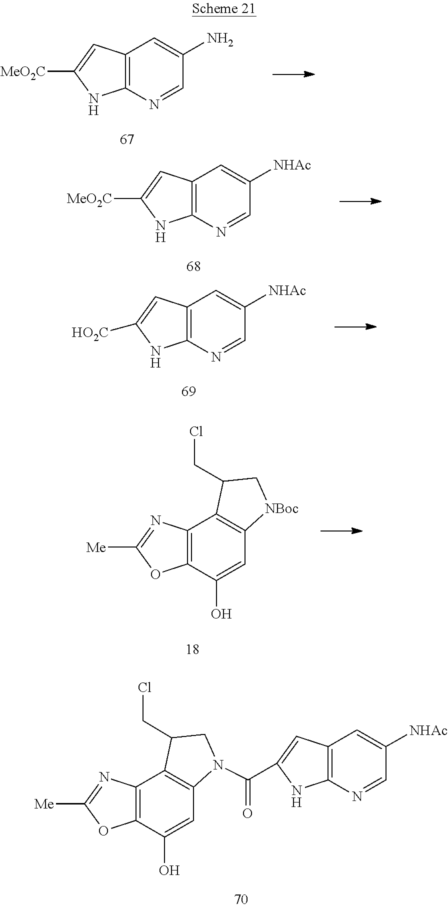 [SMILES: COC(=O)c1cc2cc(N)cnc2[nH]1 ; COC(=O)c1cc2cc(NC(C)=O)cnc2[nH]1 ; CC(=O)Nc1cnc2[nH]c(C(=O)O)cc2c1 ; Cc1nc2c3c(cc(O)c2o1)N(C(=O)OC(C)(C)C)CC3CCl ; CC(=O)Nc1cnc2[nH]c(C(=O)N3CC(CCl)c4c3cc(O)c3oc(C)nc43)cc2c1]

Acetyl chloride (0.12 mL, 1.7 mmol) was added to a mixture of methyl 5-amino-1H-pyrrolo[2,3-b]pyridine-2-carboxylate (67) (165 mg, 0.86 mmol) and Et₃N (0.36 mL, 2.6 mmol) in CH₂Cl₂ (8 mL) and THF (10 mL) at 0° C. The ice bath was removed and the mixture was stirred for 1 h and then diluted with water. The organic solvents were evaporated leaving an aqueous suspension. The solid was filtered off and dried to give methyl 5-acetamido-1H-pyrrolo[2,3-b]pyridine-2-carboxylate (68) as a white solid (178 mg, 89%); ¹H NMR (d₆-DMSO) δ 12.42 (s, 1H), 10.07 (s, 1H), 8.42 (s, 2H), 7.15 (s, 1H), 3.87 (s, 3H), 2.07 (s, 3H). Anal (C₁₁H₁₁N₃O₃) calculated for [M+H]⁺ 234.0873; found 234.0868.

A solution of KOH (205 mg, 3.6 mmol) in water (3 mL) was added to a suspension of 68 (167 mg, 0.72 mmol) in MeOH (6 mL) and the mixture was heated at reflux for 2 min and then cooled to room temperature. Aqueous HCl (2N, 1.6 mL) was added and the precipitated solid was filtered off and dried to give 5-acetamido-1H-pyrrolo[2,3-b]pyridine-2-carboxylic acid (69) as a cream solid (134 mg, 85%); ¹H NMR (d₆-DMSO) δ 13.08 (br s, 1H), 12.21 (s, 1H), 10.04 (s, 1H), 8.40 (d, J=2.3 Hz, 1H), 8.38 (d, J=2.3 Hz, 1H), 7.06 (d, J=2.0 Hz, 1H), 2.07 (s, 3H). Anal (C₁₀H₉N₃O₃) calculated for [M+H]⁺ 220.0717; found 220.0712.

A mixture of 18 (39 mg, 0.12 mmol) and HCl in dioxane (4M, 2 mL) was stirred at room temperature for 1.5 h and then evaporated. The residue was suspended in DMA (1.0 mL) and 69 (25 mg, 0.12 mmol), EDCI.HCl (66 mg, 0.36 mmol) and toluenesulfonic acid (15 mg, 0.09 mmol) were added. The mixture was stirred at room temperature for 3 h. Water was added slowly and the precipitated solid was filtered off, washed with water, and dried. The crude product was triturated with EtOAc to give 70 as a brown solid (31 mg, 61%); ¹H NMR (d₆-DMSO) δ 12.15 (s, 1H), 10.47 (s, 1H), 10.06 (s, 1H), 8.42 (d, J=2.4 Hz, 1H), 8.40 (d, J=2.3 Hz, 1H), 7.87 (br s, 1H), 7.11 (d, J=2.1 Hz, 1H), 4.74 (t, J=10.1 Hz, 1H), 4.40 (dd, J=10.9, 5.1 Hz, 1H), 4.20-4.08 (m, 2H), 4.05-3.96 (m, 1H), 2.62 (s, 3H), 2.08 (s, 3H); ¹³C NMR (d₆-DMSO) δ 168.4, 164.8, 159.7, 144.7, 141.3, 141.0, 139.5, 138.6, 136.3, 132.0, 129.8, 120.1, 118.7, 111.2, 103.8, 101.5, 54.6, 46.8, 40.7, 23.7, 14.2. Anal (C₂₁H₁₈ClN₅O₄) calculated for [M+H]⁺ 440.1120; found 440.1123.

### Example 16. N-(2-(8-(Chloromethyl)-4-hydroxy-2-methyl-7,8-dihydro-6H-oxazolo[4,5-e]indole-6-carbonyl)imidazo[1,2-a]pyridin-6-yl)acetamide (74)

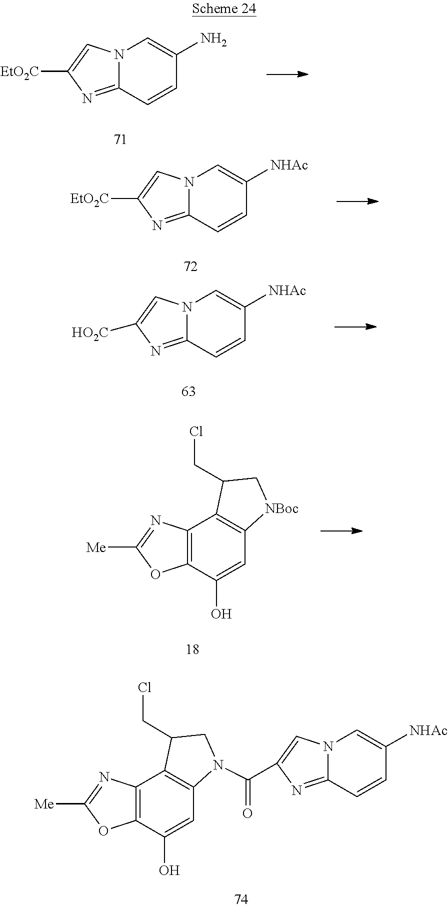 [SMILES: CCOC(=O)c1cn2cc(N)ccc2n1 ; CCOC(=O)c1cn2cc(NC(C)=O)ccc2n1 ; CC(=O)Nc1ccc2nc(C(=O)O)cn2c1 ; Cc1nc2c3c(cc(O)c2o1)N(C(=O)OC(C)(C)C)CC3CCl ; CC(=O)Nc1ccc2nc(C(=O)N3CC(CCl)c4c3cc(O)c3oc(C)nc43)cn2c1]

Acetyl chloride (0.11 mL, 1.6 mmol) was added to a mixture of ethyl 6-aminoimidazo[1,2-a]pyridine-2-carboxylate (71) (161 mg, 0.78 mmol) and Et₃N (0.33 mL, 2.3 mmol) in CH₂Cl₂ (10 mL) at room temperature. After stirring for 5 min the mixture was diluted with water and the organic solvent was evaporated, leaving an aqueous suspension. The solid was filtered off and dried, and then triturated with EtOAc, to give ethyl 6-acetamidoimidazo[1,2-a]pyridine-2-carboxylate (72) as a light green solid (99 mg, 51%); ¹H NMR (d₆-DMSO) δ 10.14 (s, 1H), 9.25 (s, 1H), 8.62 (s, 1H), 7.58 (d, J=9.7 Hz, 1H), 7.24 (dd, J=9.7, 2.0 Hz, 1H), 4.29 (q, J=7.1 Hz, 2H), 2.08 (s, 3H), 1.31 (t, J=7.1 Hz, 3H). Anal (C₁₂H₁₃N₃O₃) calculated for [M+H]⁺ 248.1030; found 248.1021.

A solution of KOH (122 mg, 2.2 mmol) in water (3 mL) was added to a suspension of 72 (96 mg, 0.39 mmol) in MeOH (5 mL) and the mixture was stirred at room temperature for 4 h. The MeOH was evaporated and the aqueous remainder was acidified with HCl (2N, 1.0 mL). The precipitated solid was filtered off and dried to give 6-acetamidoimidazo[1,2-a]pyridine-2-carboxylic acid (73) as a light brown solid (71 mg, 83%); ¹H NMR (d₆-DMSO) δ 10.13 (s, 1H), 9.25 (s, 1H), 8.55 (s, 1H), 7.58 (d, J=9.7 Hz, 1H), 7.23 (dd, J=9.7, 2.0 Hz, 1H), 2.09 (s, 3H) (CO₂H proton not observed). Anal (C₁₀H₉N₃O₃) calculated for [M+H]⁺ 220.0717; found 220.0709.

A mixture of 18 (43 mg, 0.13 mmol) and HCl in dioxane (4M, 1 mL) was stirred at room temperature for 3.5 h and then evaporated. The residue was suspended in DMA (1.0 mL) and 73 (27.8 mg, 0.13 mmol), EDCI.HCl (73 mg, 0.39 mmol) and toluenesulfonic acid (4.4 mg, 0.03 mmol) were added. The mixture was stirred at room temperature for 3 h. Water was added slowly, giving an emulsion. Aqueous NaHCO₃ was added and the mixture was extracted with EtOAc (×4). The combined extracts were washed with water and brine, and then dried and evaporated. The residue was triturated with EtOAc to give 74 as an off-white solid (25.5 mg, 46%); ¹H NMR (d₆-DMSO) δ 10.41 (s, 1H), 10.15 (s, 1H), 9.29 (d, J=1.0 Hz, 1H), 8.58 (s, 1H), 7.95 (br s, 1H), 7.67 (d, J=9.7 Hz, 1H), 7.25 (dd, J=9.7, 2.0 Hz, 1H), 4.96-4.87 (m, 1H), 4.66 (dd, J=12.2, 5.0 Hz, 1H), 4.16-4.06 (m, 2H), 4.01-3.93 (m, 1H), 2.60 (s, 3H), 2.10 (s, 3H); ¹³C NMR (d₆-DMSO) δ 168.6, 164.7, 161.4, 141.4, 141.2, 140.8, 138.6, 136.2, 127.2, 121.8, 118.6, 117.8, 116.0, 111.1, 101.5, 54.8, 46.9, 40.7, 23.7, 14.2 (one C not observed). Anal (C₂₁H₁₈ClN₅O₄) calculated for [M+H]⁺ 440.1120; found 440.1139.

### Example 17. 4-Acetamido-N-(2-(8-(chloromethyl)-4-hydroxy-2-methyl-7,8-dihydro-6H-oxazolo[4,5-e]indole-6-carbonyl)-1H-indol-5-yl)-1-methyl-1H-imidazole-2-carboxamide (79)

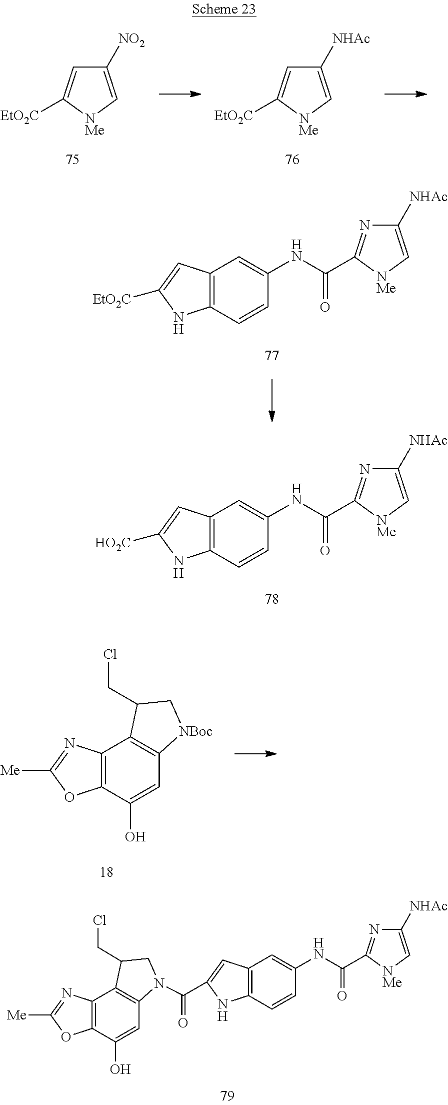 [SMILES: CCOC(=O)c1cc([N+](=O)[O-])cn1C ; CCOC(=O)c1cc(NC(C)=O)cn1C ; CCOC(=O)c1cc2cc(NC(=O)c3nc(NC(C)=O)cn3C)ccc2[nH]1 ; CC(=O)Nc1cn(C)c(C(=O)Nc2ccc3[nH]c(C(=O)O)cc3c2)n1 ; Cc1nc2c3c(cc(O)c2o1)N(C(=O)OC(C)(C)C)CC3CCl ; CC(=O)Nc1cn(C)c(C(=O)Nc2ccc3[nH]c(C(=O)N4CC(CCl)c5c4cc(O)c4oc(C)nc54)cc3c2)n1]

Pd/C (10%, 90 mg) was added to a solution of ethyl 1-methyl-4-nitro-1H-pyrrole-2-carboxylate (75) (254 mg, 1.28 mmol) in EtOH (25 mL) and the mixture was hydrogenated at 50 psi for 2 h. The mixture was filtered through Celite, washing with EtOAc, and the filtrate was evaporated. The residue was dissolved in CH₂Cl₂ (6 mL) and the solution was cooled in an ice bath. Et₃N (0.54 mL, 3.8 mmol) and acetyl chloride (0.18 mL, 2.6 mmol) were added and the ice bath was removed. The mixture was stirred for 10 min and then diluted with water and extracted with CH₂Cl₂ (×2). The extracts were washed with dilute aqueous HCl and water, and then dried and evaporated. The residue was recrystallized from EtOH to give ethyl 4-acetamido-1-methyl-1H-pyrrole-2-carboxylate (76) as a pale yellow solid (65 mg, 24%); mp 165-168° C. The aqueous phases from the liquid-liquid extraction were basified with aqueous NaHCO₃ and extracted with EtOAc (×4). The extracts were dried, combined with the mother liquor from the recrystallisation, and evaporated. The residue was purified by column chromatography (EtOAc:petroleum ether 1:1 then 4:1 then EtOAc only) to give more 76 (139 mg, 52%); ¹H NMR (CDCl₃) δ 7.91 (br s, 1H), 7.47 (s, 1H), 4.41 (q, J=7.1 Hz, 2H), 3.99 (s, 3H), 2.14 (s, 3H), 1.42 (t, J=7.1 Hz, 3H). Anal (C₉H₁₃N₃O₃) calculated for [M+H]⁺ 212.1030; found 212.1026.

A solution of KOH (218 mg, 3.9 mmol) in water (2 mL) was added to a solution of 76 (185 mg, 0.88 mmol) in MeOH (4 mL) and the mixture was stirred at room temperature for 30 min. The MeOH was evaporated and the aqueous layer was neutralised with aqueous HCl (2N, 1.3 mL) and then evaporated to dryness. Aminoindole 60 (179 mg, 0.88 mmol), EDCI.HCl (0.50 g, 2.6 mmol) and DMA (2 mL) were added and the mixture was stirred at room temperature for 29 h. The mixture was diluted with water and extracted with EtOAc (×2). The extracts were washed with water (×3) and then dried and evaporated. The residue was triturated with EtOAc to give ethyl 5-(4-acetamido-1-methyl-1H-imidazole-2-carboxamido)-1H-indole-2-carboxylate (77) as a cream solid (59 mg, 18% over 2 steps); ¹H NMR (d₆-DMSO) δ 11.85 (s, 1H), 10.36 (s, 1H), 9.74 (s, 1H), 8.13 (d, J=1.7 Hz, 1H), 7.49 (dd, J=8.9, 2.0 Hz, 1H), 7.47 (s, 1H), 7.41 (d, J=8.9 Hz, 1H), 7.12 (d, J=1.3 Hz, 1H), 4.33 (q, J=7.1 Hz, 2H), 3.97 (s, 3H), 2.03 (s, 3H), 1.34 (t, J=7.1 Hz, 3H). Anal (C₁₈H₁₉N₅O₄) calculated for [M+H]⁺ 370.1510; found 270.1505.

A solution of KOH (89 mg, 1.6 mmol) in water (1 mL) was added to a suspension of 77 (54 mg, 0.15 mmol) in MeOH (8 mL) and the mixture was stirred at 50° C. for 3 h and then at reflux for 1.5 h. The mixture was cooled, the MeOH was evaporated, and the aqueous residue was acidified with aqueous HCl (2N, 0.8 mL). The precipitated solid was filtered off and dried to give 5-(4-acetamido-1-methyl-1H-imidazole-2-carboxamido)-1H-indole-2-carboxylic acid (78) as a grey solid (46 mg, 92%); ¹H NMR (d₆-DMSO) δ 12.95 (br s, 1H), 11.71 (s, 1H), 10.37 (s, 1H), 9.72 (s, 1H), 8.11 (s, 1H), 7.50-7.43 (m, 2H), 7.39 (d, J=8.9 Hz, 1H), 7.06 (d, J=1.6 Hz, 1H), 3.97 (s, 3H), 2.02 (s, 3H). Anal (C₁₆H₅N₅O₄) calculated for [M+H]⁺ 342.1197; found 342.1204.

A mixture of 18 (39.4 mg, 0.12 mmol) and HCl in dioxane (4M, 1 mL) was stirred at room temperature for 3.5 h and then evaporated. The residue was suspended in DMA (1.0 mL) and 78 (39.7 mg, 0.12 mmol), EDCI.HCl (67 mg, 0.36 mmol) and toluenesulfonic acid (4 mg, 0.02 mmol) were added. The mixture was stirred at room temperature for 5 h. Water was added slowly, and the precipitated solid was filtered off, washed with water, and dried. The crude product was triturated with EtOAc to give 79 as an off-white solid (36 mg, 55%); ¹H NMR (d₆-DMSO) δ 11.71 (s, 1H), 10.46 (s, 1H), 10.37 (s, 1H), 9.75 (s, 1H), 8.12 (d, J=1.7 Hz, 1H), 7.92 (s, 1H), 7.54 (dd, J=8.9, 2.0 Hz, 1H), 7.48-7.43 (m, 2H), 7.15 (d, J=1.7 Hz, 1H), 4.80 (t, J=10.2 Hz, 1H), 4.46 (dd, J=10.9, 5.1 Hz, 1H), 4.22-4.10 (m, 2H), 4.05-3.99 (m, 1H), 3.97 (s, 3H), 2.63 (s, 3H), 2.04 (s, 3H); ¹³C NMR (d₆-DMSO) δ 167.2, 164.8, 159.8, 156.8, 141.3, 141.2, 138.6, 136.3, 134.0, 133.3, 131.4, 131.0, 127.1, 118.8, 114.0, 112.4, 112.3, 111.1, 105.5, 101.6, 54.5, 46.9, 40.8, 35.0, 22.7, 14.2. Anal (C₂₇H₂₄ClN₇O₅) calculated for [M+H]⁺ 562.1600; found 562.1579.

### Example 18. (S)-(8-(Chloromethyl)-4-hydroxy-2-methyl-7,8-dihydro-6H-oxazolo[4,5-e]indol-6-yl)(5,6,7-trimethoxy-1H-indol-2-yl)methanone (82) and (R)-(8-(chloromethyl)-4-hydroxy-2-methyl-7,8-dihydro-6H-oxazolo[4,5-e]indol-6-yl)(5,6,7-trimethoxy-1H-indol-2-yl)methanone (83)

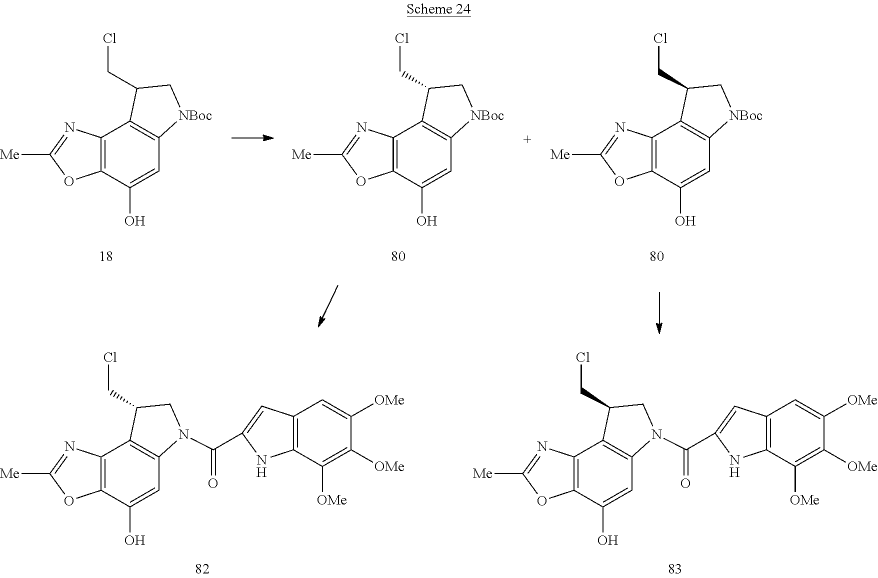 [SMILES: Cc1nc2c3c(cc(O)c2o1)N(C(=O)OC(C)(C)C)CC3CCl ; Cc1nc2c3c(cc(O)c2o1)N(C(=O)OC(C)(C)C)C[C@H]3CCl ; Cc1nc2c3c(cc(O)c2o1)N(C(=O)OC(C)(C)C)C[C@@H]3CCl ; COc1cc2cc(C(=O)N3C[C@@H](CCl)c4c3cc(O)c3oc(C)nc43)[nH]c2c(OC)c1OC ; COc1cc2cc(C(=O)N3C[C@H](CCl)c4c3cc(O)c3oc(C)nc43)[nH]c2c(OC)c1OC]

Compound 18 (334 mg) was resolved by preparative chiral HPLC (Daicel Chiralpak IA 250×21 mm column, EtOH:hexane 15:85 eluant, 6 mL/min flow rate, a 1.43) giving baseline separation of the two enantiomers. The fractions containing the faster-eluting enantiomer (R_{T} 9.4 min) were combined and evaporated to give tert-butyl (S)-8-(chloromethyl)-4-hydroxy-2-methyl-7,8-dihydro-6H-oxazolo[4,5-e]indole-6-carboxylate (80) as a white solid (131 mg, 39%); [α]_{D}=−31° (c 0.584, CH₂Cl₂); ¹H NMR (CDCl₃) identical to that described for 18. The fractions containing the slower-eluting enantiomer (R_{T} 13.4 min) were combined and evaporated to give tert-butyl (R)-8-(chloromethyl)-4-hydroxy-2-methyl-7,8-dihydro-6H-oxazolo[4,5-e]indole-6-carboxylate (81) as a white solid (130 mg, 39%); [α]_{D}=+24° (c 0.586, CH₂Cl₂); ¹H NMR (CDCl₃) identical to that described for 18. Reanalysis on an analytical column (Daicel Chiralpak IA 150×4.6 mm column) confirmed 100% ee for each enantiomer.

Compound 80 was converted to 82 by the general method described to provide a white solid. Anal (C₂₃H₂₂ClN₃O₆) calculated for [M+H]⁺ 472.12699; found 472.12721. Compound 81 was converted to 83 by the general method described to provide a white solid; ¹H NMR (d₆-DMSO) identical to that described for 23. Anal (C₂₃H₂₂ClN₃O₆) calculated for [M+H]⁺ 472.12699; found 472.12719.

### Example 19. 8-(Chloromethyl)-2-methyl-6-(5,6,7-trimethoxy-1H-indole-2-carbonyl)-7,8-dihydro-6H-oxazolo[4,5-e]indol-4-yl (4-((S)-2-((S)-2-(6-(2,5-dioxo-2,5-dihydro-1H-pyrrol-1-yl)hexanamido)-3-methylbutanamido)-5-ureidopentanamido)benzyl) ethane-1,2-diylbis(methylcarbamate) (26)

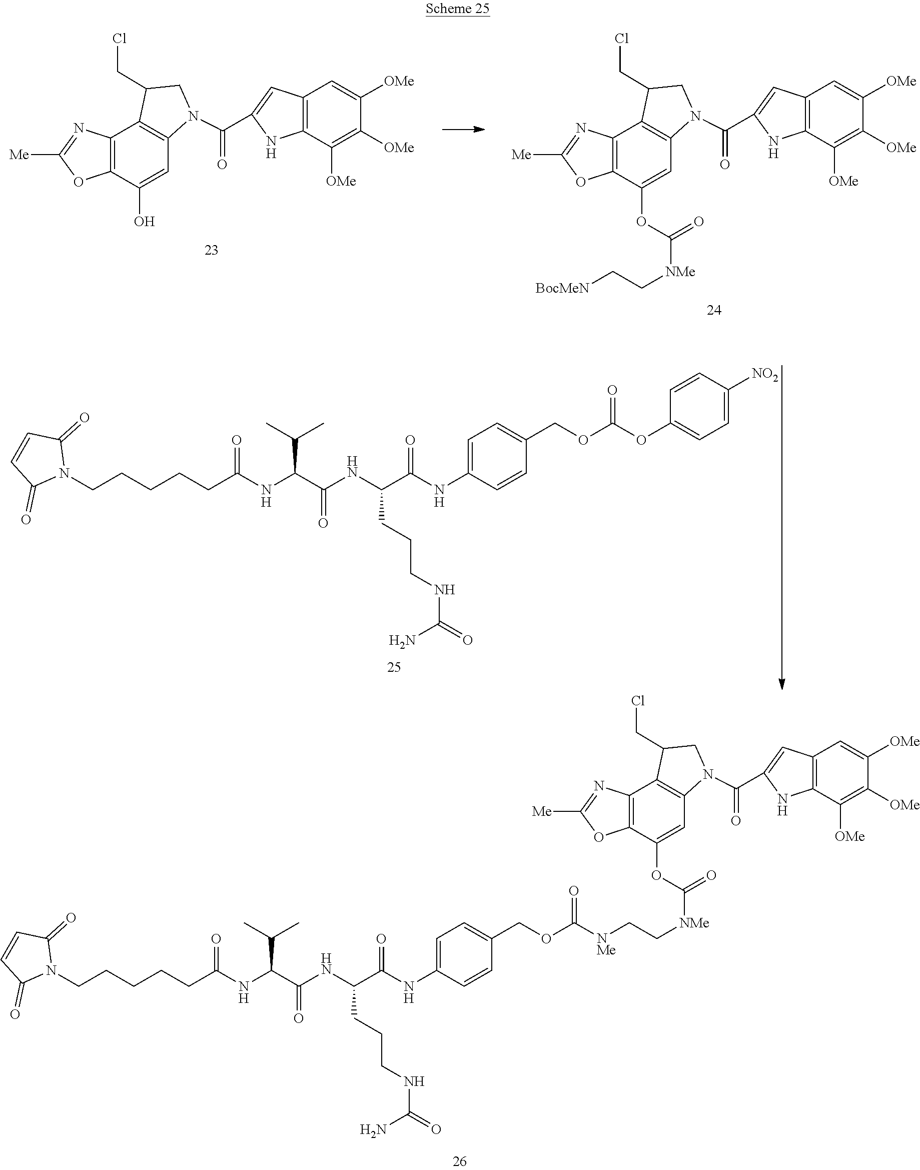

p-Nitrophenyl chloroformate (97%, 20 mg, 0.10 mmol) and Et₃N (34 μL, 0.25 mmol) were added to a solution of 23 (23 mg, 0.049 mmol) in dry THF (5 mL) and DMF (1.5 mL) at 0° C. After 1.5 h more p-nitrophenyl chloroformate (97%, 10 mg, 0.05 mmol) and Et₃N (17 μL, 0.12 mmol) were added, and after a further 50 min tert-butyl methyl(2-(methylamino)ethyl)carbamate (95%, 40 mg, 0.21 mmol) was added. The ice bath was removed and the mixture was stirred for a further 20 h, then evaporated to dryness. The residue was purified by column chromatography (EtOAc:petroleum ether 1:4 then 1:3, 1:1, 3:1) to give tert-butyl (8-(chloromethyl)-2-methyl-6-(5,6,7-trimethoxy-1H-indole-2-carbonyl)-7,8-dihydro-6H-oxazolo[4,5-e]indol-4-yl) ethane-1,2-diylbis(methylcarbamate) (24) as a colourless oil (31 mg, 94%); ¹H NMR (CDCl₃) (some signals split due to rotamers) δ 9.37 (s, 1H), 8.24 and 8.23 (2s, 1H), 6.97 (d, J=2.3 Hz, 1H), 6.87 (s, 1H), 4.77 (t, J=9.6 Hz, 1H), 4.61 (dd, J=10.7, 4.6 Hz, 1H), 4.29-4.21 (m, 2H), 4.091 and 4.090 (2s, 3H), 3.95 (s, 3H), 3.92 (s, 3H), 3.76-3.34 (m, 5H), 3.21 and 3.08 (2s, 3H), 3.01-2.84 (m, 3H), 2.65 (s, 3H), 1.53-1.42 (m, 9H). Anal (C₃₃H₄₀ClN₅O₉) calculated for [M+H]⁺ 686.2587; found 686.2573.

TFA (0.7 mL, 9.1 mmol) was added to a solution of 24 (31 mg, 0.045 mmol) in CH₂Cl₂ (0.7 mL) at 0° C. After 30 min the mixture was evaporated to dryness at room temperature and the residue was dissolved in CH₂Cl₂ and evaporated again. The residue was dissolved in DMF (0.5 mL) and the solution was cooled in an ice bath. 4-((S)-2-((S)-2-(6-(2,5-Dioxo-2,5-dihydro-1H-pyrrol-1-yl)hexanamido)-3-methylbutanamido)-5-ureidopentanamido)benzyl (4-nitrophenyl) carbonate (25) (36.7 mg, 0.050 mmol) and Et₃N (32 μL, 0.23 mmol) were added. The ice bath was removed and the mixture was stirred for 18 h, then evaporated to dryness at room temperature. The residue was purified by column chromatography (CH₂Cl₂ only then CH₂Cl₂:MeOH 100:1, then 50:1, 30:1, 15:1) to give recovered 23 (6 mg, 28%) and crude product. The latter was purified by column chromatography (EtOAc:MeOH 9:1) to give 26 as a pale yellow glass (22 mg, 41%); ¹H NMR (d₆-DMSO) (some signals split due to rotamers) δ 11.48-11.37 (m, 1H), 9.99-9.90 (m, 1H), 8.08 and 8.06 (2s, 1H), 8.05-7.97 (m, 1H), 7.81 and 7.79 (2s, 1H), 7.60-7.46 (m, 2H), 7.36-7.16 (m, 2H), 7.07-7.02 (m, 1H), 7.00 (s, 2H), 6.96 (s, 1H), 5.96 (t, J=5.5 Hz, 1H), 5.40 (s, 2H), 5.08-4.95 (m, 2H), 4.81-4.72 (m, 1H), 4.48-4.25 (m, 3H), 4.21-4.06 (m, 3H), 3.93 (s, 3H), 3.32 (s, 3H), 3.28 (s, 3H), 3.67-3.44 (m, 4H), 3.36 (t, J=7.1 Hz, 2H), 3.14-2.85 (m, 8H), 2.67-2.57 (m, 3H), 2.23-2.06 (m, 2H), 2.01-1.90 (m, 1H), 1.74-1.31 (m, 8H), 1.24-1.13 (m, 2H), 0.87-0.76 (m, 6H). Anal (C₅₇H₇₀ClN₁₁O₁₅) calculated for [M+H]⁺ 1184.4814; found 1184.4840.

### Example 20. 8-(Chloromethyl)-2-methyl-6-(5,6,7-trimethoxy-1H-indole-2-carbonyl)-7,8-dihydro-6H-oxazolo[4,5-e]indol-4-yl (2-((((4-((S)-2-((S)-2-((tert-butoxycarbonyl)amino)-3-methylbutanamido)propanamido)benzyl)oxy)carbonyl)(methyl)amino)ethyl)(2-(2-hydroxyethoxy)ethyl)carbamate (87)

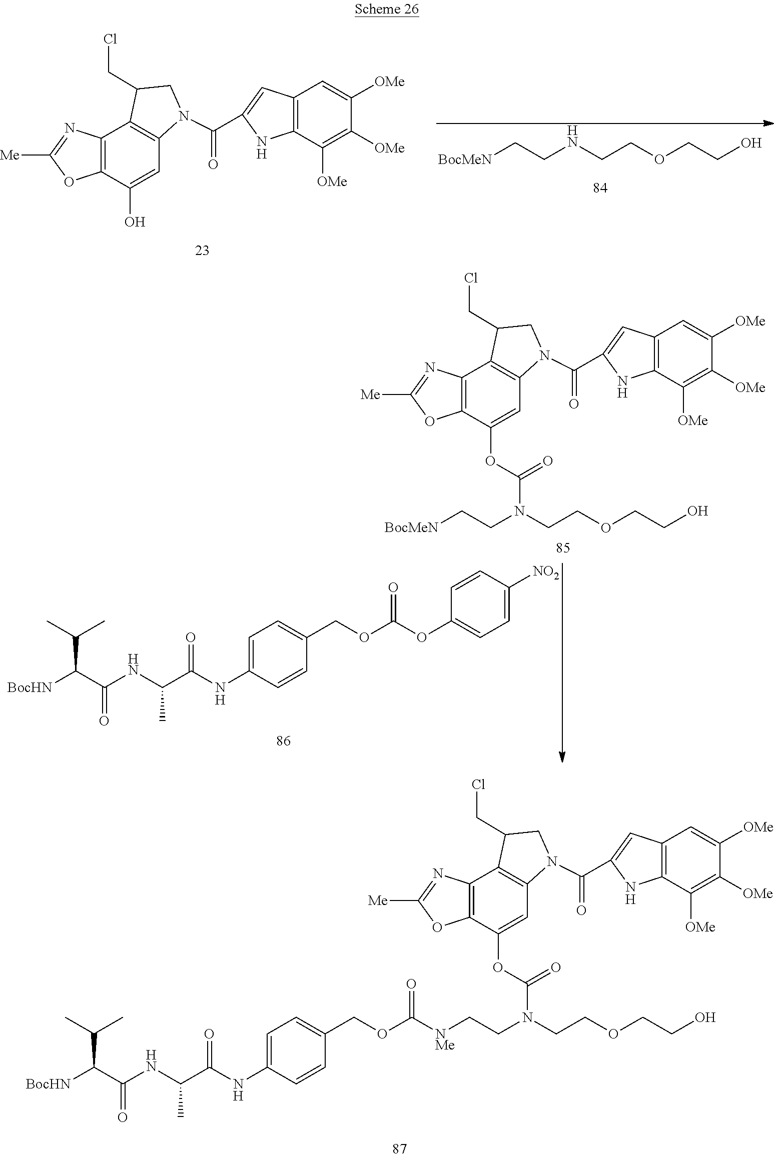

p-Nitrophenyl chloroformate (97%, 39 mg, 0.18 mmol) and Et₃N (64 μL, 0.46 mmol) were added to a solution of 23 (43.5 mg, 0.092 mmol) in DMF (4 mL) at 0° C. Further portions of p-nitrophenyl chloroformate (97%, 19 mg, 0.18 mmol) were added after 45 min and 2 h, and more Et₃N (32 μL, 0.28 mmol) was added after 70 min. After 2.5 h a solution of tert-butyl (2-((2-(2-hydroxyethoxy)ethyl)amino)ethyl)(methyl)carbamate (84) (73 mg, 0.28 mmol) in DMF (1 mL) was added and the ice bath was removed. The mixture was stirred for 18 h and then evaporated to dryness. The residue was triturated with CH₂Cl₂ and the solid was filtered off and dried to give recovered 23 (23 mg, 53%). The filtrate was evaporated and the residue was purified by column chromatography (EtOAc:petroleum ether 1:1 then 3:1, then EtOAc only, then EtOAc:MeOH 20:1) to give 8-(chloromethyl)-2-methyl-6-(5,6,7-tri methoxy-1H-indole-2-carbonyl)-7,8-dihydro-6H-oxazolo[4,5-e]indol-4-yl (2-((tert-butoxycarbonyl)(methyl)amino)ethyl)(2-(2-hydroxyethoxy)ethyl)carbamate (85) as a colourless oil (20 mg, 29%); ¹H NMR (CDCl₃) (some signals split due to rotamers) δ 9.39 (s, 1H), 8.36, 8.34 and 8.29 (3s, 1H), 6.97 (d, J=2.3 Hz, 1H), 6.87 (s, 1H), 4.82-4.74 (m, 1H), 4.64-4.57 (m, 1H), 4.29-4.20 (m, 2H), 4.09 (s, 3H), 3.95 (s, 3H), 3.92 (s, 3H), 3.85-3.44 (m, 14H), 2.99-2.86 (m, 3H), 2.66 (s, 3H), 1.54-1.42 (m, 9H). Anal (C₃₆H₄₆ClN₅O₁₁) calculated for [M+H]⁺ 760.2955; found 760.2940.

Compound 85 (31 mg, 0.041 mmol) was treated with a mixture of CH₂Cl₂ (0.7 mL) and TFA (0.7 mL, 9.1 mmol) at 0° C. for 15 min. The mixture was evaporated to dryness at room temperature and the residue was dissolved in CH₂Cl₂ and evaporated again. The residue was dissolved in DMF (0.5 mL) and the solution was cooled in an ice bath. tert-Butyl ((S)-3-methyl-1-(((S)-1-((4-((((4-nitrophenoxy)carbonyl)oxy)methyl)phenyl)amino)-1-oxopropan-2-yl)amino)-1-oxobutan-2-yl)carbamate (86) (23 mg, 0.041 mmol) and Et₃N (28 μL, 0.21 mmol) were added and the ice bath was removed. After 2.5 h the mixture was evaporated and the residue was purified by column chromatography (EtOAc:petroleum ether 2:3 then 3:2, then EtOAc only, then EtOAc:MeOH 20:1) to give recovered 23 (4.3 mg, 22%) and 87 as a colourless glass (24.8 mg, 56%); ¹H NMR (CDCl₃) (some signals split due to rotamers) δ 9.64-9.44 (m, 1H), 8.96-8.74 (m, 1H), 8.28-8.02 (m, 1H), 7.63-7.46 (m, 2H), 7.36-7.23 (m, 1H), 6.97 (s, 1H), 6.90-6.80 (m, 2H), 5.19-5.06 (m, 2H), 4.80-4.72 (m, 1H), 4.67-4.56 (m, 2H), 4.28-4.20 (m, 2H), 4.08 (s, 3H), 4.02-3.93 (m, 1H), 3.94 (s, 3H), 3.91 (s, 3H), 3.84-3.41 (m, 13H), 3.05-2.95 (m, 3H), 2.66-2.61 (m, 3H), 2.21-2.11 (m, 1H), 1.44 (s, 12H), 0.98-0.89 (m, 6H). Anal (C₅₂H₆₇ClN₈O₁₅) calculated for [M+H]⁺ 1079.4487; found 1079.4489.

### Example 21. 8-(Chloromethyl)-6-(5-(2-(dimethylamino)ethoxy)-1H-indole-2-carbonyl)-2-methyl-7,8-dihydro-6H-oxazolo[4,5-e]indol-4-yl (2-((((4-((S)-2-((S)-2-((tert-butoxycarbonyl)amino)-3-methylbutanamido)propanamido)benzyl)oxy)carbonyl)(methyl)amino)ethyl)(2-(2-hydroxyethoxy)ethyl)carbamate (89)

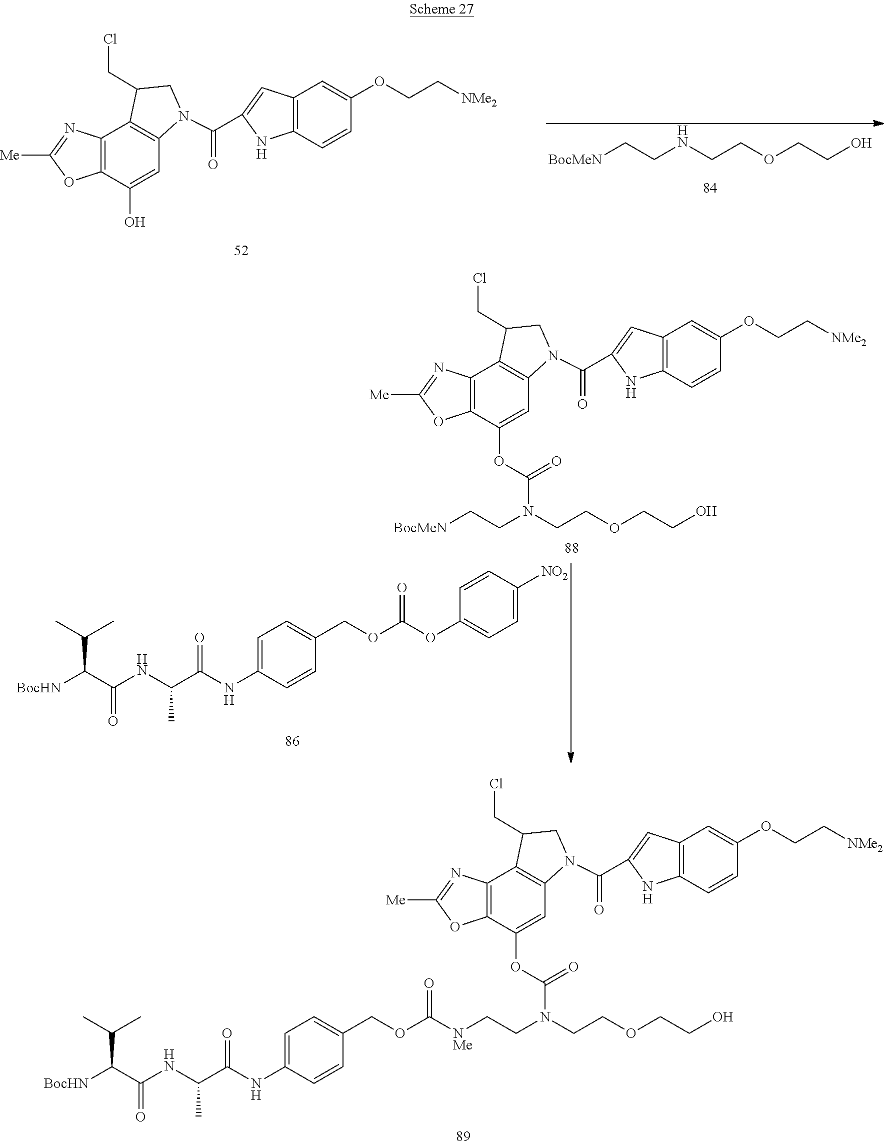

p-Nitrophenyl chloroformate (97%, 34.5 mg, 0.16 mmol) and Et₃N (88 μL, 0.63 mmol) were added to a solution of 52 (59.3 mg, 0.126 mmol) in THF (6 mL) and DMF (1.5 mL) at 0° C. After 30 min more p-nitrophenyl chloroformate (97%, 34.5 mg, 0.16 mmol) was added, and after 2.5 h a solution of 84 (66 mg, 0.32 mmol) in THF (1 mL) was added. The ice bath was removed and the mixture was stirred for 18 h and then evaporated to dryness. The residue was triturated with CH₂Cl₂ and the solid was filtered off and dried to give recovered 52 as the hydrochloride salt (21 mg, 32%). The filtrate was evaporated and the residue was purified by column chromatography (EtOAc only then EtOAc:MeOH 10:1 then 5:1 then 3:2) to give 8-(chloromethyl)-6-(5-(2-(dimethylamino)ethoxy)-1H-indole-2-carbonyl)-2-methyl-7,8-dihydro-6H-oxazolo[4,5-e]indol-4-yl (2-((tert-butoxycarbonyl)(methyl)amino)ethyl)(2-(2-hydroxyethoxy)ethyl)carbamate (88) as a colourless oil (30 mg, 31%); ¹H NMR (CDCl₃) (some signals split due to rotamers) δ 9.52-9.41 (m, 1H), 8.26 and 8.23 (2s, 1H), 7.34 (d, J=8.9 Hz, 1H), 7.14 (d, J=2.2 Hz, 1H), 7.03 (dd, J=8.9, 2.3 Hz, 1H), 6.97 (d, J=1.4 Hz, 1H), 4.81-4.74 (m, 1H), 4.64-4.57 (m, 1H), 4.28-4.16 (m, 4H), 3.83-3.41 (m, 14H), 2.97-2.85 (m, 5H), 2.65 (s, 3H), 2.45 (s, 6H), 1.52-1.42 (m, 9H). Anal (C₃₇H₄₉ClN₆O₉) calculated for [M+H]⁺ 757.3322; found 757.3332.

Compound 88 (30 mg, 0.040 mmol) was treated with a mixture of CH₂Cl₂ (0.7 mL) and TFA (0.7 mL, 9.1 mmol) at 0° C. for 15 min. The mixture was evaporated to dryness at room temperature and the residue was dissolved in CH₂Cl₂ and evaporated again. The residue was dissolved in DMF (0.5 mL) and the solution was cooled in an ice bath. Compound 86 (22 mg, 0.040 mmol) and Et₃N (33 μL, 0.24 mmol) were added and the ice bath was removed. After stirring for 2 h the mixture was evaporated and the residue was purified by column chromatography (CH₂Cl₂:MeOH 50:1 then 20:1, 10:1, 8:1; followed by a second column eluting with EtOAc:MeOH 10:1 then 5:1, 3:1, 3:2) to give 89 as a colourless glass (7.8 mg, 18%); ¹H NMR (CDCl₃) (some signals split due to rotamers) δ 10.01-9.47 (m, 1H), 8.95-8.61 (m, 1H), 8.27-8.06 (m, 1H), 7.61-7.44 (m, 2H), 7.41-7.19 (m, 2H), 7.14 (d, J=2.2 Hz, 1H), 7.06-6.95 (m, 2H), 5.19-4.99 (m, 2H), 4.83-4.73 (m, 1H), 4.71-4.57 (m, 2H), 4.28-4.17 (m, 2H), 4.14 (t, J=5.7 Hz, 2H), 4.02-3.92 (m, 1H), 3.83-3.34 (m, 14H), 3.05-2.93 (m, 3H), 2.80 (t, J=5.7 Hz, 2H), 2.67-2.61 (m, 3H), 2.38 (s, 6H), 2.23-2.14 (m, 1H), 1.47-1.38 (m, 12H), 1.00-0.90 (m, 6H). Anal (C₅₃H₇₀ClN₉O₁₃) calculated for [M+H]⁺ 1076.4854; found 1076.4857.

### Example 22. 8-(Chloromethyl)-6-(5-(2-methoxyethoxy)-1H-indole-2-carbonyl)-2-methyl-7,8-dihydro-6H-oxazolo[4,5-e]indol-4-yl (2-((((4-((S)-2-((S)-2-((tert-butoxycarbonyl)amino)-3-methylbutanamido)propanamido)benzyl)oxy)carbonyl)(methyl)amino)ethyl)(2-(2-hydroxyethoxy)ethyl)carbamate (91)

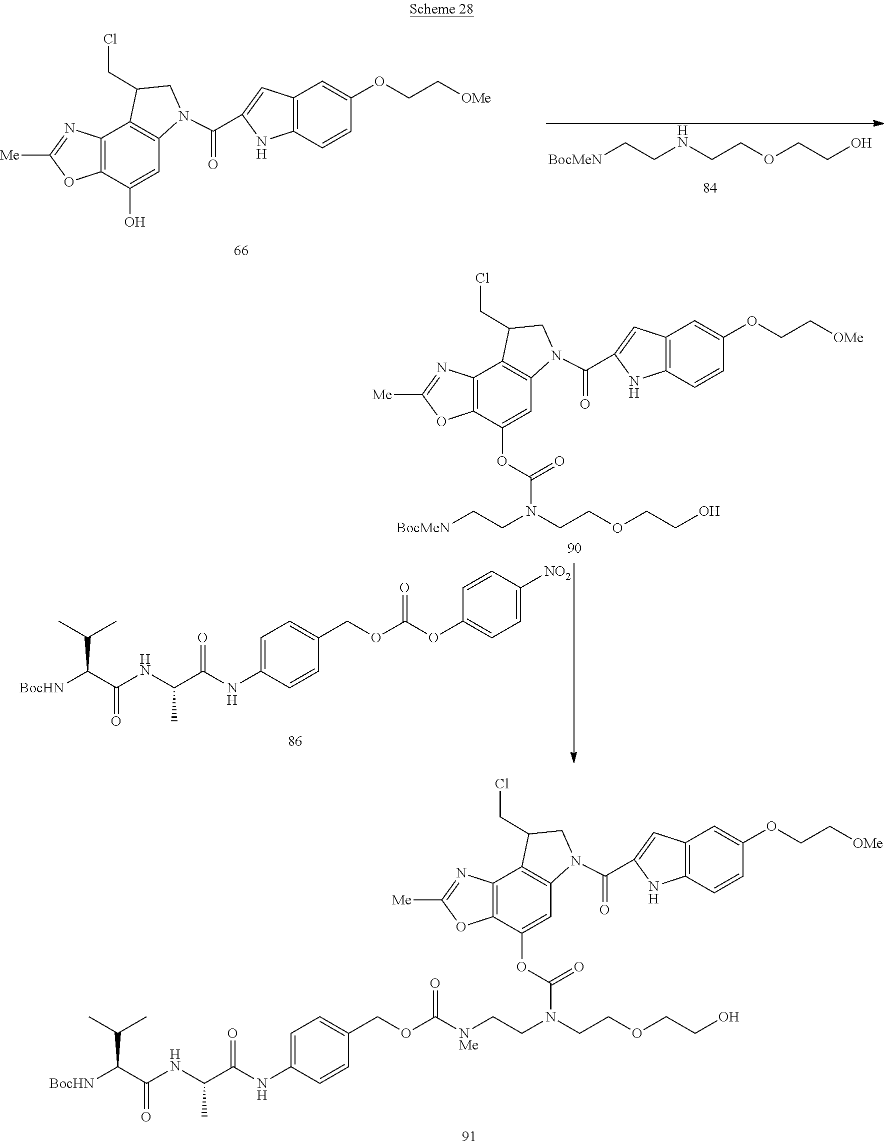

p-Nitrophenyl chloroformate (96%, 160 mg, 0.76 mmol) and Et₃N (0.37 mL, 2.7 mmol) were added to a solution of 66 (242 mg, 0.53 mmol) in THF (8 mL) at 0° C. After 75 min a solution of 84 (279 mg, 1.1 mmol) in THF (1.5 mL) was added. The ice bath was removed and the mixture was stirred for 5.5 h and then evaporated to dryness. The residual yellow-brown foam was dissolved in the minimum CH₂Cl₂ and the solution was allowed to stand at 4° C. overnight. The solid was filtered off, washing with CH₂Cl₂, and the filtrate was evaporated. The residue was purified by column chromatography (EtOAc:petroleum ether 1:1 then 3:1, then EtOAc only, then EtOAc:MeOH 20:1) to give 8-(chloromethyl)-6-(5-(2-methoxyethoxy)-1H-indole-2-carbonyl)-2-methyl-7,8-dihydro-6H-oxazolo[4,5-e]indol-4-yl (2-((tert-butoxycarbonyl)(methyl)amino)ethyl)(2-(2-hydroxyethoxy)ethyl)carbamate (90) as a colourless oil (90 mg, 23%); ¹H NMR (CDCl₃) (some signals split due to rotamers) δ 9.43 (s, 1H), 8.26 and 8.24 (2s, 1H), 7.34 (d, J=8.9 Hz, 1H), 7.14 (d, J=2.3 Hz, 1H), 7.05 (dd, J=8.9, 2.4 Hz, 1H), 6.98 (d, J=1.5 Hz, 1H), 4.81-4.74 (m, 1H), 4.65-4.57 (m, 1H), 4.28-4.13 (m, 4H), 3.85-3.51 (m, 15H), 3.48 (s, 3H), 2.98-2.85 (m, 3H), 2.65 (s, 3H), 1.53-1.42 (m, 9H). Anal (C₃₆H₄₆ClN₅O₁₀) calculated for [M+H]⁺ 744.3006; found 744.3014.

The solid that was insoluble in CH₂Cl₂ was combined with matching fractions from column chromatography to give recovered 66 (142 mg, 59%).

TFA (0.7 mL, 9.1 mmol) was added to a solution of 90 (90 mg, 0.12 mmol) in CH₂Cl₂ (2.0 mL) at 0° C. and the mixture was stirred at this temperature for 40 min. The mixture was evaporated to dryness at room temperature and the residue was dissolved in CH₂Cl₂ and evaporated again. The residue was dissolved in DMF (1 mL) and the solution was cooled in an ice bath. Compound 86 (67.5 mg, 0.12 mmol) and Et₃N (84 μL, 0.6 mmol) were added and the ice bath was removed. After 3 h the mixture was evaporated and the residue was purified by column chromatography (EtOAc:petroleum ether 2:3 then 1:1, 3:1, then EtOAc only, then EtOAc:MeOH 50:1 then 25:1, 15:1) to give recovered 66 as a white solid (11 mg, 20%) and 91 as a white glass (58 mg, 45%); ¹H NMR (CDCl₃) (some signals split due to rotamers) δ 9.95-9.40 (m, 1H), 8.91-8.55 (m, 1H), 8.27-8.05 (m, 1H), 7.61-7.45 (m, 2H), 7.40-7.20 (m, 2H), 7.15 (d, J=2.2 Hz, 1H), 7.06 (dd, J=8.9, 2.2 Hz, 1H), 7.00 (s, 1H), 6.72-6.53 (m, 1H), 5.21-4.94 (m, 3H), 4.84-4.74 (m, 1H), 4.72-4.56 (m, 2H), 4.28-4.16 (m, 4H), 4.01-3.92 (m, 1H), 3.84-3.35 (m, 15H), 3.47 (s, 3H), 3.06-2.94 (m, 3H), 2.66-2.60 (m, 3H), 2.24-2.13 (m, 1H), 1.48-1.37 (m, 12H), 1.01-0.90 (m, 6H). Anal (C₅₂H₆₇ClN₈O₁₄) calculated for [M+H]⁺ 1063.4538; found 1063.4546.

A further fraction of less pure 91 was also obtained from the column as a white glass (14 mg, crude yield 11%).

### Example 23. 8-(Chloromethyl)-6-(5-(2-methoxyethoxy)-1H-indole-2-carbonyl)-2-methyl-7,8-dihydro-6H-oxazolo[4,5-e]indol-4-yl (2-((((4-((2S,5S)-13-(2,5-dioxo-2,5-dihydro-1H-pyrrol-1-yl)-5-isopropyl-2-methyl-4,7-dioxo-8,11-dioxa-3,6-diazatridecanamido)benzyl)oxy)carbonyl)(methyl)amino)ethyl)(2-(2-hydroxyethoxy)ethyl)carbamate (93)

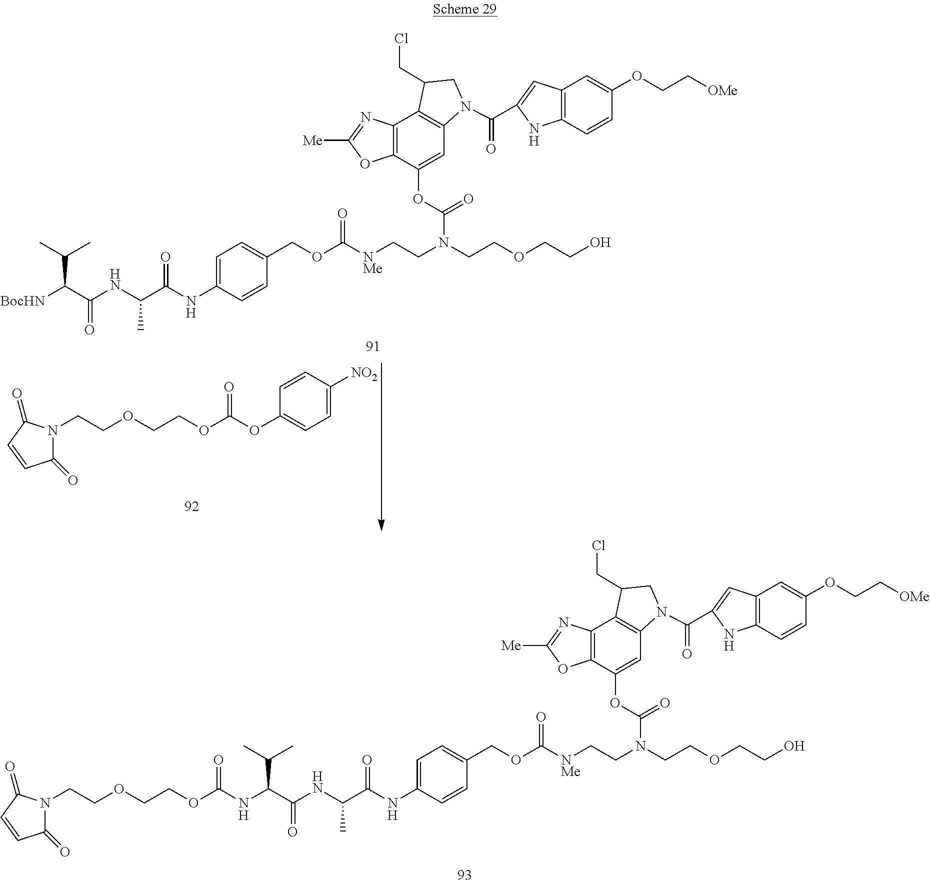 [SMILES: COCCOc1ccc2[nH]c(C(=O)N3CC(CCl)c4c3cc(OC(=O)N(CCOCCO)CCN(C)C(=O)OCc3ccc(NC(=O)[C@H](C)NC(=O)[C@@H](NC(=O)OC(C)(C)C)C(C)C)cc3)c3oc(C)nc43)cc2c1 ; O=C(OCCOCCN1C(=O)C=CC1=O)Oc1ccc([N+](=O)[O-])cc1 ; COCCOc1ccc2[nH]c(C(=O)N3CC(CCl)c4c3cc(OC(=O)N(CCOCCO)CCN(C)C(=O)OCc3ccc(NC(=O)[C@H](C)NC(=O)[C@@H](NC(=O)OCCOCCN5C(=O)C=CC5=O)C(C)C)cc3)c3oc(C)nc43)cc2c1]

TFA (0.2 mL, 2.6 mmol) was added to a solution of 91 (14 mg, 0.013 mmol) in CH₂Cl₂ (1.0 mL) at 0° C. and the solution was kept at this temperature for 3 h. The mixture was evaporated to dryness at room temperature and the residue was dissolved in CH₂Cl₂ and evaporated again. The residue was dissolved in DMF (0.5 mL) and the solution was cooled in an ice bath. 2-(2-(2,5-Dioxo-2,5-dihydro-1H-pyrrol-1-yl)ethoxy)ethyl (4-nitrophenyl) carbonate (92) (4.6 mg, 0.013 mmol) and Et₃N (9 μL, 0.065 mmol) were added and the ice bath was removed. After 19 h the mixture was evaporated and the residue was purified by column chromatography (EtOAc only, then EtOAc:MeOH 20:1) to give 93 as a colourless oil (8.3 mg, 54%); ¹H NMR (CDCl₃) (some signals split due to rotamers, 2 exchangeable protons not observed) δ 9.94-9.46 (m, 1H), 8.87-8.57 (m, 1H), 8.31-8.00 (m, 1H), 7.63-7.46 (m, 2H), 7.43-7.22 (m, 2H), 7.16-6.93 (m, 4H), 6.68 (s, 2H), 5.40-5.32 (m, 1H), 5.16-5.04 (m, 2H), 4.83-4.56 (m, 3H), 4.35-3.98 (m, 7H), 3.83-3.36 (m, 21H), 3.47 (s, 3H), 3.05-2.94 (m, 3H), 2.66-2.60 (m, 3H), 2.30-2.20 (m, 1H), 1.46-1.36 (m, 3H), 1.03-0.90 (m, 6H). Anal (C₅₆H₆₈ClN₉O₁₇) calculated for [M+H]⁺ 1174.4494; found 1174.4516.

### Example 24. Additional Lipophilicity Data

Lipophilicity of small molecule compounds can be calculated in many different ways (reviewed for example in J. Pharm. Sci. (2009) 98, 861). To complement the data provided in Example 6 the calculated lipophilicity of several compounds of the present invention was compared to that of the seco-CBI analogues bearing the same minor groove binding side chain, using four different software packages, i.e. ChemDraw Professional (Perkin Elmer Informatics Inc) as in Example 6, ACD/log P (Advanced Chemistry Development), XLOGP3 (Shanghai Institute of Organic Chemistry), and MLOGP (Talete SRL, Milano, Italy), with the last three accessed via SwissADME (http://swissadme.ch/, Swiss Institute of Bioinformatics). The structures of the compounds are shown (where DNA binding units A, D, E and F, when attached to the 2-methylbenzoxazole alkylating subunit, represent compounds 23, 59, 66 and 74, respectively) and the calculated log P values are collected in Table 3.

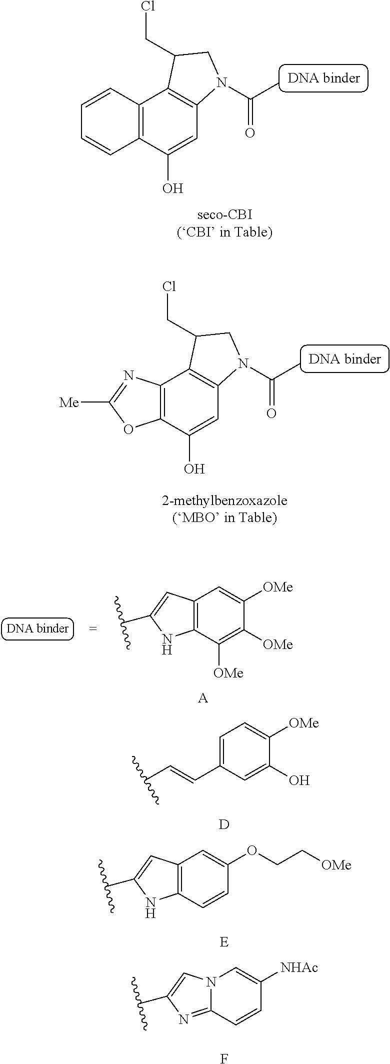

It is clear from Table 3 that although the absolute log P values vary depending on the calculation programme used, in every case there is a consistent and substantial reduction in log P when comparing the 2-methylbenzoxazole alkylating subunit to the seco-CBI alkylating subunit (Δ log P in the range 0.98-1.48). This applies whether the calculation programme is based on properties (e.g. MLOGP) or on substructures (either fragment-based e.g. ACD, or atom-based e.g. XLOG3).

As already noted, these substantial reductions in payload lipophilicity are highly likely to lead to favourable properties for drug-linkers and ADCs which incorporate the 2-methylbenzoaxazole alkylating subunit.

TABLE 3
Lipophilicity of compounds of the invention

| | LogP | | | | | | | | | | | |
|---|---|---|---|---|---|---|---|---|---|---|---|---|
| DNA- | ChemDraw | | | ACD | | | XLOGP3 | | | MLOGP | | |
| binder | CBI | MBO | Δ | CBI | MBO | Δ | CBI | MBO | Δ | CBI | MBO | Δ |
| A | 3.36 | 1.88 | 1.48 | 4.20 | 2.53 | 1.67 | 4.63 | 3.62 | 1.01 | 2.44 | 1.44 | 1.00 |
| D | 3.96 | 2.49 | 1.48 | 4.27 | 2.66 | 1.61 | 4.27 | 3.29 | 0.98 | 2.91 | 1.85 | 1.06 |
| E | 3.45 | 1.98 | 1.48 | 5.30 | 3.63 | 1.67 | 4.54 | 3.53 | 1.01 | 2.70 | 1.68 | 1.02 |
| F | 2.33 | 0.85 | 1.48 | 3.86 | 2.20 | 1.66 | 3.60 | 2.59 | 1.01 | 2.23 | 1.22 | 1.01 |

### Example 25. Additional Cytotoxicity Data

The cytotoxicity of the payloads of the invention was determined by measuring the inhibition of proliferation of two human tumour cell lines, the cervical carcinoma SiHa, and the ovarian carcinoma SKOV3, following the general procedure described in Example 7. In the new determinations the stock solution of payload in DMSO was diluted in series prior to addition to the cell-containing wells of the 96-well plate. The cytotoxicity determinations were repeated several times (n=3-7 depending on compound and cell line) and the results are collected in Table 4.

The new data reinforce the information presented in Table 2 in showing that DNA alkylating agents containing the 2-methylbenzoxazole alkylating subunit can be highly cytotoxic compounds, with IC₅₀s in the nM or sub-nM range, i.e. within the range considered suitable for ADC application. They also show that appropriate choice of the minor groove binding side chain can be used to modulate the cytotoxicity of the payload, with the examples in Table 4 spanning more than a 100-fold range in IC₅₀ in the 2 cell lines examined. The data in Table 4 further allow a comparison between the two enantiomeric forms of the 2-methylbenzoxazole alkylating subunit. Compounds 82 and 83 show a differential cytotoxic potency of 93-fold in SiHa and 110-fold in SKOV3. These observations are consistent with the behaviour of other analogues of the duocarmycins in that, although both enantiomers are cytotoxic as a consequence of alkylating DNA, the natural enantiomer is generally more cytotoxic.

Importantly, compounds 23 and seco-CBI-TMI, which share the same minor groove binding side chain, were again found to have the same cytotoxic potency, within the error of the IC₅₀ measurement. This means that the 2-methylbenzoxazole alkylating subunit can be considered one of the most potent of all known variants of duocarmycin-type alkylating subunits, surprisingly unlike the closely-related seco-COI alkylating subunit.

TABLE 4
Cytotoxicity of compounds of the invention

| | IC₅₀ (nM) | | | |
|---|---|---|---|---|
| | SiHa | | SKOV3 | |
| Compound | Average | SEM | Average | SEM |
| Seco- | 0.16 | 0.033 | 0.27 | 0.018 |
| CBI-TMI | | | | |
| 23 | 0.18 | 0.031 | 0.32 | 0.059 |
| 52 | 0.44 | 0.054 | 1.17 | 0.37 |
| 53 | 2.9 | 0.57 | 4.2 | 0.93 |
| 57 | 3.6 | 0.38 | 4.3 | 0.75 |
| 59 | 0.62 | 0.10 | 0.50 | 0.044 |
| 63 | 0.69 | 0.24 | 0.61 | 0.097 |
| 65 | 2.7 | 1.0 | 1.7 | 0.37 |
| 66 | 0.20 | 0.005 | 0.17 | 0.034 |
| 70 | >20 | na | >20 | na |
| 74 | 13 | 4.1 | 16.4 | 2.0 |
| 82 | 0.15 | 0.014 | 0.30 | 0.019 |
| 83 | 14 | 1.8 | 33 | 0.81 |

### Example 26. Additional Stability Data

The aqueous stability of compounds 52, 59 and 66 was investigated by HPLC analysis. The conditions were the same as those described in Example 8, i.e. samples contained the payloads at a concentration of 4 μM in Tris buffer (pH 7.4) containing 10% DMF at 37° C. In this experiment the solutions were monitored at hourly intervals over 8 h.

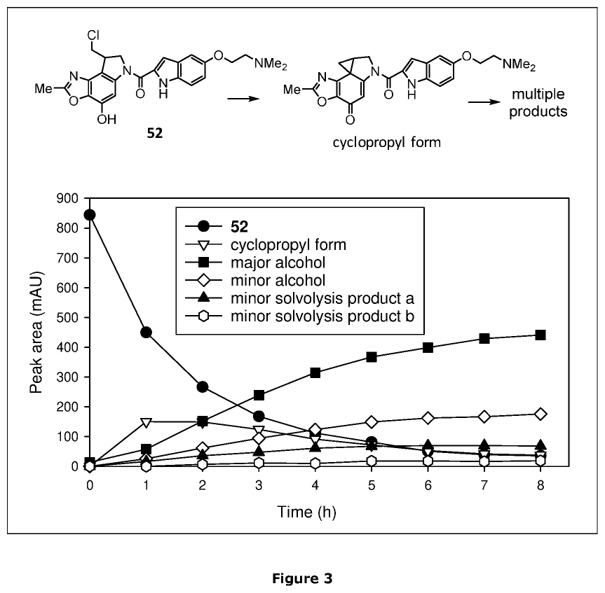

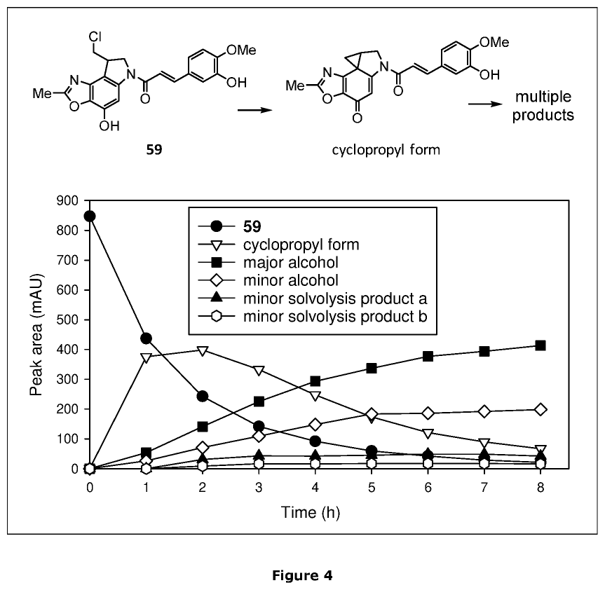

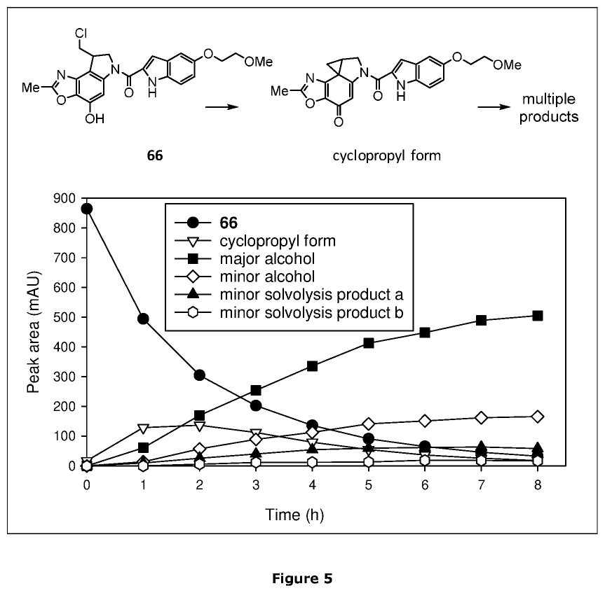

As shown in FIGS. 3-5, all three payload compounds were converted to an intermediate that was identified as the corresponding cyclopropyl form on the basis of a characteristic shift in UV-Vis absorption spectra. In all three cases the cyclopropyl intermediate was unstable towards further reaction in aqueous buffer, generating a variety of products with two major hydrolysis products predominating. This behaviour closely matches the stability and product distribution observed for compound 23 in Example 8 (see FIG. 2) but stands in marked contrast to the prolonged stability of reference compound seco-CBI-TMI (see FIG. 1). Therefore, it appears that the unusual instability is a general property of the 2-methylbenzoxazole alkylating subunit. It does not depend markedly on the nature of the DNA minor groove binding side chain. In other words, the potential advantage of hydrolysis as a detoxifying mechanism for any systemically released payload can be reasonably assumed to be a property common to all the new compounds of this invention.

1. A compound of formula I or a pharmaceutically acceptable salt, hydrate or solvate thereof 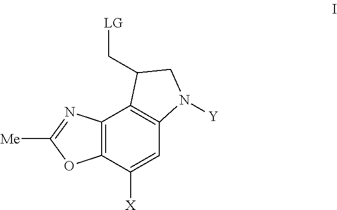
wherein:
LG is a leaving group;
X is a group selected from hydroxyl, protected hydroxyl, prodrug hydroxyl, amino, and protected amino; where amino is —NH₂, or —NH(C₁-C₆)alkyl; and
Y is a N-protecting group.

2. A compound of claim 1 wherein Y is a N-protecting group selected from Boc, COCF₃, Fmoc, Alloc, Cbz, Teoc and Troc.

3. A compound formula II or a pharmaceutically acceptable salt, hydrate or solvate thereof 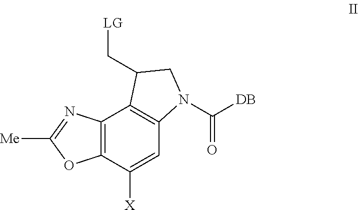
wherein:
LG is a leaving group;
X is a group selected from hydroxyl, protected hydroxyl, prodrug hydroxyl, amino, and protected amino; where amino is —NH₂, or —NH(C₁-C₆)alkyl; and
DB is a DNA minor groove binding unit.

4. A compound of claim 3 wherein DB is an optionally substituted aryl or optionally substituted heteroaryl group attached directly or via an alkenyl group, or an optionally substituted indole, azaindole, benzene, benzofuran, pyridine, pyrimidine, pyrrole, imidazole, thiophene, thiazole, oxazole, pyrazole, triazole, pyrazine or pyridazine group.

5. (canceled)

6. A compound of claim 3 wherein X is selected from the group consisting of —OH, —OBn, —OTf, —OMOM, —OMEM, —OBOM, —OTBDMS, —OPMB, —OSEM, piperazine-1-carboxylate where the N at the 4 position is substituted with (C₁-C₁₀)alkyl, —OP(O)(OH)₂, —OP(O)(OR²)₂, —NH₂, —N═C(Ph)₂, —NHZ, NH(C₁-C₁₀)alkyl and —N—Z(C₁-C₁₀)alkyl;
wherein R² is t-Bu, Bn or allyl; and Z is selected from Boc, COCF₃, Fmoc, Alloc, Cbz, Teoc and Troc.

7. A compound of claim 3 wherein LG is selected from the group consisting of chloride, bromide, iodide and —OSO₂R¹; wherein R¹ is selected from (C₁-C₁₀)alkyl, (C₁-C₁₀)heteroalkyl, (C₁-C₁₀)aryl or (C₁-C₁₀)heteroaryl.

8. A compound of formula III or a pharmaceutically acceptable salt, hydrate or solvate thereof 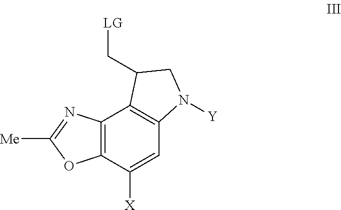
wherein:
LG is a leaving group;
X is a group selected from hydroxyl, protected hydroxyl, prodrug hydroxyl, amino, and protected amino; where amino is —NH₂, or —NH(C₁-C₆)alkyl; and
Y is selected from:
(a) a N-protecting group;
(b) —C(O)—Ar¹
(c) —C(O)—Ar¹—NH—C(O)—Ar²
(d) —C(O)—Ar¹—NH—C(O)—CH═CH—Ar³; or
(e) —C(O)—CH═CH—Ar³
where Ar¹, Ar² and Ar³ are each independently selected from a heteroaryl or aryl group, where the heteroaryl or aryl group is optionally substituted with one or more of —(C₁-C₆)alkyl, —CO—(C₁-C₆)alkyl, —CONH(C₁-C₆)alkyl, —CON(C₁-C₆)alkyl(C₁-C₆)alkyl, —OH, —O—(C₁-C₆)alkyl, —NH₂, —NH(C₁-C₆)alkyl, —N(C₁-C₆)alkyl(C₁-C₆)alkyl and —NHC(O)—(C₁-C₆)alkyl;
wherein in each instance —(C₁-C₆)alkyl, —CO—(C₁-C₆)alkyl, —CONH(C₁-C₆)alkyl, —CON(C₁-C₆)alkyl(C₁-C₆)alkyl, —O—(C₁-C₆)alkyl, —NH(C₁-C₆)alkyl, —N(C₁-C₆)alkyl(C₁-C₆)alkyl and —NHC(O)—(C₁-C₆)alkyl are independently optionally substituted with one or more of —NMe₂, —NHMe, —NH₂, —OH, morpholine and —SH.

9. A compound of claim 8 wherein Y is an N-protecting group selected from the group consisting of Boc, COCF₃, Fmoc, Alloc, Cbz, Teoc and Troc.

10. A compound of claim 8 wherein Y is selected from the group consisting of:
(b) —C(O)—Ar¹
(c) —C(O)—Ar¹—NH—C(O)—Ar²
(d) —C(O)—Ar¹—NH—C(O)—CH═CH—Ar³; and
(e) —C(O)—CH═CH—Ar³
wherein
Ar¹, Ar² and Ar³ are independently selected from the group consisting of 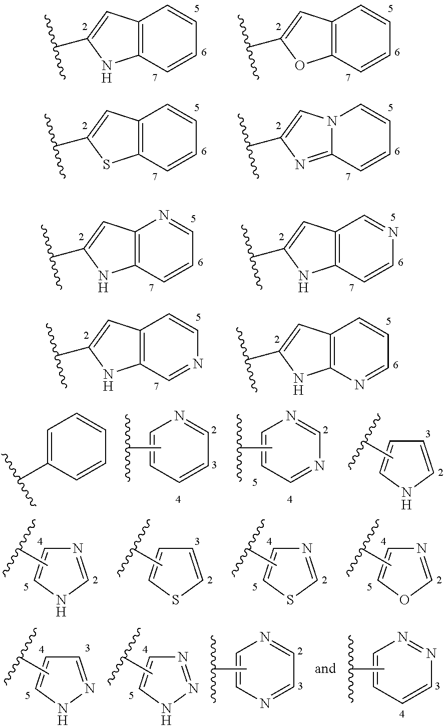
where [symbol] represents the point of attachment and each of the aryl or heteroaryl groups may be substituted at the numbered positions with up to three substituents selected from —(C₁-C₆)alkyl, —CO—(C₁-C₆)alkyl, —CONH(C₁-C₆)alkyl, —CON(C₁-C₆)alkyl(C₁-C₆)alkyl, —OH, —O—(C₁-C₆)alkyl, —NH₂, —NH(C₁-C₆)alkyl, —N(C₁-C₆)alkyl(C₁-C₆)alkyl and —NHC(O)—(C₁-C₆)alkyl, and wherein in each instance —(C₁-C₆)alkyl, —CO—(C₁-C₆)alkyl, —CONH(C₁-C₆)alkyl, —CON(C₁-C₆)alkyl(C₁-C₆)alkyl, —O—(C₁-C₆)alkyl, —NH(C₁-C₆)alkyl, —N(C₁-C₆)alkyl(C₁-C₆)alkyl and —NHC(O)—(C₁-C₆)alkyl are independently optionally substituted with one or more of —NMe₂, —NHMe, —NH₂, —OH, morpholine and —SH.

11. A compound of claim 8 wherein Ar¹ is a heteroaryl group, preferably an indole, azaindole, benzofuran or benzothiophene group, which is connected to the DNA alkylating unit at the 2-position of the heteroaryl group.

12. A compound of claim 8 wherein Ar² is selected from the group consisting of indole, azaindole, benzene, benzofuran, pyridine, pyrimidine, pyrrole, imidazole, thiophene, thiazole, oxazole, pyrazole, triazole, pyrazine or pyridazine, preferably indole, azaindole, benzene, benzofuran, pyrrole or imidazole.

13. A compound of claim 8 wherein Ar³ is benzene, pyridine, pyrimidine or pryridazine.

14. A compound of claim 8 wherein
(a) Y is —C(O)—Ar¹—NH—C(O)—Ar²;
Ar¹ is an indole, azaindole, benzofuran or benzothiophene group, which is connected to the DNA alkylating unit at the 2-position of the heteroaryl group and Ar² is selected from the group consisting of indole, azaindole, benzene, benzofuran, pyridine, pyrimidine, pyrrole, imidazole, thiophene, thiazole, oxazole, pyrazole, triazole, pyrazine or pyridazine; or
(b) Y is —C(O)—Ar¹—NH—C(O)—CH═CH—Ar³; wherein Ar¹ is an indole, azaindole, benzofuran or benzothiophene group, which is connected to the DNA alkylating unit at the 2-position of the heteroaryl group and Ar³ is selected from benzene, pyridine, pyrimidine and pyridazine.

15.-16. (canceled)

17. A compound of claim 8 wherein X is selected from the group consisting of —OH, —OBn, —OTf, —OMOM, —OMEM, —OBOM, —OTBDMS, —OPMB, —OSEM, piperazine-1-carboxylate where the N at the 4 position is substituted with (C₁-C₁₀)alkyl, —OP(O)(OH)₂, —OP(O)(OR²)₂, —NH₂, —N═C(Ph)₂, —NHZ, NH(C₁-C₁₀)alkyl or —N—Z(C₁-C₁₀)alkyl;
wherein R² is t-Bu, Bn or allyl; and Z is selected from Boc, COCF₃, Fmoc, Alloc, Cbz, Teoc and Troc.

18. A compound of claim 8 wherein LG is selected from the group consisting of chloride, bromide, iodide and —OSO₂R¹; wherein R¹ is selected from (C₁-C₁₀)alkyl, (C₁-C₁₀)heteroalkyl, (C₁-C₁₀)aryl or (C₁-C₁₀)heteroaryl.

19. A compound of claim 8 wherein LG is halo, and the configuration at the chiral carbon to which LG is attached is (S).

20. A compound of claim 8 being selected from the group consisting of:

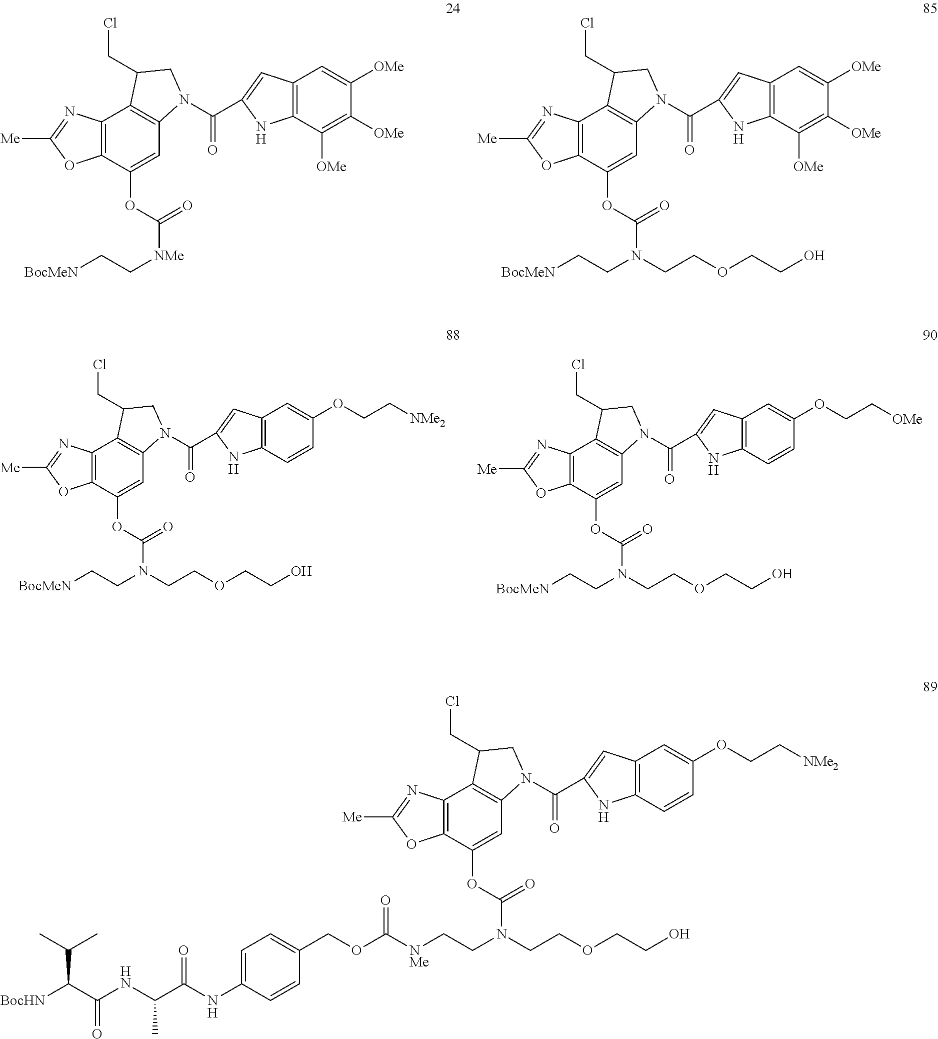

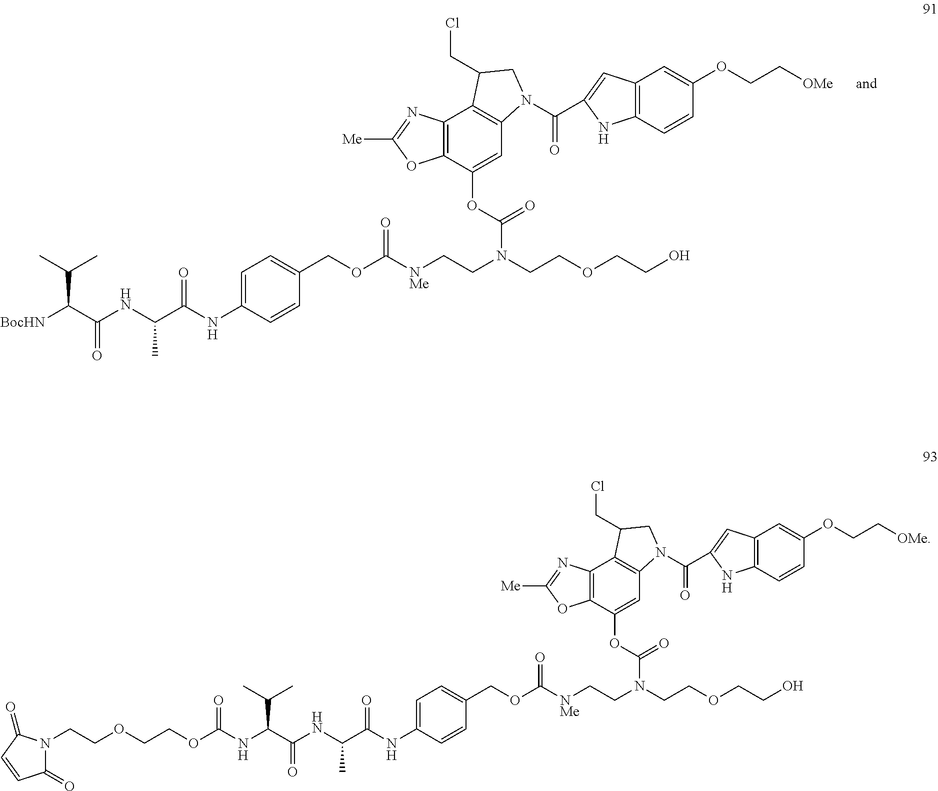 [SMILES: COCCOc1ccc2[nH]c(C(=O)N3CC(CCl)c4c3cc(OC(=O)N(CCOCCO)CCN(C)C(=O)OCc3ccc(NC(=O)[C@H](C)NC(=O)[C@@H](NC(=O)OC(C)(C)C)C(C)C)cc3)c3oc(C)nc43)cc2c1 ; COCCOc1ccc2[nH]c(C(=O)N3CC(CCl)c4c3cc(OC(=O)N(CCOCCO)CCN(C)C(=O)OCc3ccc(NC(=O)[C@H](C)NC(=O)[C@@H](NC(=O)OCCOCCN5C(=O)C=CC5=O)C(C)C)cc3)c3oc(C)nc43)cc2c1]

21. A pharmaceutical composition comprising a compound of claim 8 and a pharmaceutically acceptable carrier.

22. A compound of claim 3 wherein DB comprises a reactive moiety RM which is compatible with a complementary reactive site on a linker group, or a component of a linker group, wherein said linker group is attached to, or is suitable for attachment to a ligand.

23. A compound of claim 8 being bound to an antibody via a linker group.
